(19) United States
(12) Patent Application Publication
(10) Pub. No.: US 2016/0235869 A1
(43) Pub. Date: Aug. 18, 2016

(54) DERIVATIVES OF DIBENZOTHIOPHENE IMAGING OF alpha-7 NICOTINIC ACETYLCHOLINE RECEPTORS

(71) Applicant: THE JOHNS HOPKINS UNIVERSITY, BALTIMORE, MD (US)

(72) Inventors: Yongjun Gao, Baltimore, MD (US); Andrew Horti, Ellicott City, MD (US); Robert Dannals, Baltimore, MD (US); Richard Wahl, Baltimore, MD (US); Ronnie Mease, Fairfax, VA (US); Ken Kellar, Washington, DC (US); Paige Finley, Baltimore, MD (US); Martin Pomper, BALTIMORE, MD (US)

(73) Assignee: THE JOHNS HOPKINS UNIVERSITY, BALTIMORE, MD (US)

(21) Appl. No.: 14/622,373

(22) Filed: Feb. 13, 2015

(51) Int. Cl. A61K 51/04; C07B 59/00

(52) U.S. Cl. CPC A61K 51/0468; C07B 59/002

(57) ABSTRACT

The presently disclosed subject matter provides non-invasive methods for imaging, quantifying α7 nicotinic cholinergic receptors, and diagnosing a disease or condition associated with α7-nAChRs. Methods for preparing radiolabeled derivatives of dibenzothiophene and compounds provided thereof also are provided.

### FEDERALLY SPONSORED RESEARCH OR DEVELOPMENT

This invention was made with government support under MH079017 and AG037298 awarded by the National Institutes of Health. The government has certain rights in the invention.

### BACKGROUND

Cerebral neuronal nicotinic cholinergic receptors (nAChRs) are ligand-gated ion channels composed of a (i.e., α2-α10) and β (i.e., β2-β4) subunits that can assemble in multiple combinations of pentameric structures. Among the many nAChRs subtypes in the human central nervous system, heteropentameric α4β2-nAChRs and homopentameric α7-nAChRs are predominant. Gotti and Clementi, Prog. Neurobiol. (2004); Lukas, et al., Pharmacol. Rev. (1999).

α7-nAChRs are composed of five identical α7 subunits, and each subunit provides an orthosteric binding site for its neurotransmitter acetylcholine. Dani and Bertrand, Annu. Rev. Pharmacol. Toxicol. (2007). Many lines of evidence associate α7-nAChRs with the pathophysiology of a variety of disorders, such as schizophrenia and Alzheimer's disease (AD), anxiety, depression, traumatic brain injury, multiple sclerosis, inflammation, and drug addiction. Philip, et al., Psychopharmacology (Berlin, Ger.) (2010); Ishikawa and Hashimoto, Curr. Pharm. Des. (2011); Parri, et al., Biochem. Pharmacol. (2011); Albuquerque, et al., Physiol. Rev. (2009); Woodruff-Pak and Gould, Behav. Cognit. Neurosci. Rev. (2002); D'Hoedt and Bertrand, Expert Opin. Ther. Targets (2009); Hoffmeister, et al., NeuroMol. Med. (2011); Verbois, et al., J. Neurotrauma (2000); Verbois, et al., J. Neurotrauma (2002).

Clinical experiments with α7-nAChR agonists have demonstrated that selective activation of the receptor is a viable approach toward improving cognitive performance in patients with schizophrenia. Olincy, A., et al., Arch. Gen. Psychiatry (2006); Thomsen, et al., Curr. Pharm. Des. (2010).

Because of the importance of the α7-nAChR in human neurophysiology and as a potential drug target, synthesis and preclinical examination of α7-nAChR subtype selective compounds receive substantial interest in industry and academia. D'Hoedt and Bertrand (2009); Thomsen, et al., Curr. Pharm. Des. (2010). A number of α7-nAChR drugs are currently in various stages of the development for treatment of a variety of disorders including schizophrenia, AD, multiple sclerosis, depression, asthma, and type 2 diabetes. Mazurov, et al., J. Med. Chem. (2011); Taly and Charon, Curr. Drug Targets (2012); Wallace and Bertrand, Expert Opin. Ther. Targets (2013).

In vivo imaging and quantification of α7-nAChR binding in humans would provide a significant advance in the understanding of α7-nAChR-related CNS disorders and also could facilitate novel α7-nAChR drug development. Positron emission tomography (PET) is the most advanced technique to quantify neuronal receptors and their occupancy in vivo, and the development of a suitable PET radiotracer for α7-nAChRs would be of particular interest. Due to its lower cost compared to PET and its availability, single-photon emission computed tomography (SPECT) is the most widely used technique to provide 3D information, and it is a better choice for imaging procedures that requires longer time. Many lead structures of α7-nAChR ligands have been identified within various structural classes. A number of these ligands have been radiolabeled for PET ([¹⁸F], [¹¹C]) and SPECT ([¹²³I] [¹²⁵I]) (Table 1) and studied in mice, pigs, and non-human primates as potential α7-nAChR probes. Pomper, et al., J. Nucl. Med. (2005); Hashimoto, et al., PLoS One (2008); Ogawa, et al., Nucl. Med. Biol. (2010); Dolle, et al., J. Labelled Compd. Radiopharm. (2001); Toyohara, et al., PLoS One (2010); Horti, et al., Nucl. Med. Biol. (2013); Gao, et al., Bioorg. Med. Chem. (2012); Toyohara, et al., Ann. Nucl. Med. (2009); Ettrup, et al., J. Nucl. Med. (2011); Ravert, et al., Nucl. Med. Biol. (2013); Rotering, et al., Bioorg. Med. Chem. (2013); Deuther-Conrad, et al., Eur. J. Nucl. Med. Mol. Imaging (2011).

Most of these radioligands entered the animal brain, but manifested relatively low specific binding (for review, see Horti and Villemagne, Curr. Pharm. Des. (2006); Toyohara, et al., Curr. Top. Med. Chem. (2010); Brust, et al., Curr. Drug Targets (2012)) and insufficient BP_{ND} values (BP_{ND}<1) (Table 1). [¹¹C]CHIBA-1001 is the only α7-nAChR PET radioligand so far that has been studied in human subjects, Toyohara, et al., Ann. Nucl. Med. (2009), but it also exhibits low specific binding (see, for example, Table 1).

Further, until now, no good α7-nAChR SPECT radioligands have become available. The most common in vitro radiotracers for α7-nAChR are labeled snake toxin peptide [¹²⁵I]α-Bgt and the alkaloid [³H]MLA. Davie et al., Neuropharmacology (1999). Both radiotracers have been invaluable tools for in vitro characterization of α7-nAChR, and yet they both exhibit substantial drawbacks.

[¹²⁵I]α-Bgt binds with muscle type nAChRs and neuronal α7-, α8- and α9-nAChRs. [¹²⁵I]α-Bgt has a large size and, consequently, may not be able to access synaptic receptors. The toxin exhibits very slow, almost irreversible binding kinetics and, in addition, its handling is not user-friendly. Davie et al., Neuropharmacology (1999). [³H]MLA exhibit more rapid binding kinetics than that of [¹²⁵I]α-Bgt. However, [³H]MLA displays a relatively high non-specific binding and moderate binding affinity. Anderson et al., J. Pharmacol. Exp. Ther. (2008).

The latest radioligand [³H]A-585539 exhibits a better binding affinity than [³H]MLA and low non-specific binding, but structurally [³H]A-585539 is a quaternary ammonium cation and intrinsically it does not penetrate the cell membranes because it is electrically charged. Anderson et al., J. Pharmacol. Exp. Ther. (2008).

Because of the exceptionally low concentration (Bₘₐₓ) of cerebral α7-nAChR binding sites in the human (5-15 fmol/mg protein), Marutle, et al., J. Chem. Neuroanat. (2001), and animal brain (1.5-12 fmol/mg tissue), Kulak and Schneider, Brain Res. (2004); Kulak, et al., Eur. J. Neurosci. (2006), a PET or SPECT radioligand with high specific brain uptake for this receptor subtype must exhibit very high binding affinity and selectivity, along with other important properties (e.g., lipophilicity, polar surface area, suitability for radiolabeling) in an appropriate range (for details, see Horti and Villemagne, Curr. Pharm. Des. (2006); Brust, et al., Curr. Drug Targets (2012); Zhang, et al., J. Med. Chem. (2013); Eckelman, et al., J. Nucl. Med. (1979).

The general aptness of a PET radioligand for quantitative imaging studies is defined by a conventional criterion Bₘₐₓ/K_{D}≧10. Eckelman, et al., J. Nucl. Med. (1979). This equation predicts that a picomolar range of the binding affinity is required for a good α7-nAChR PET radioligand (K_{D}≦0.15-1.2 nM), whereas the most previously published α7-nAChR radioligands exhibited nanomolar binding affinities (Table 1). It is noteworthy, however, that the inhibition binding assays of the published compounds have been performed under a variety of assay conditions, and thus, the values of Kᵢ listed in Table 1 may not be directly comparable to one another.

Recently, Abbott Laboratories has reported 3-(1,4-diazabicyclo[3.2.2]nonan-4-yl)dibenzo[b,d]thiophene 5,5-dioxide 5 (FIG. 1) as an α7-nAChR selective antagonist with extraordinarily high binding affinity, Kᵢ=0.023 nM. Schrimpf, et al., Bioorg. Med. Chem. Lett. (2012).

TABLE 1
In Vitro Properties and Binding Potential in Cortex (BP_{ND}) of the
Previously Published PET/SPECT Radioligands for Imaging of α7-nAChR

| | α7-nAChR, | | Monkey | |
|---|---|---|---|---|
| Radioligand | Kᵢ, nM | Mice | or Pig | References |
| 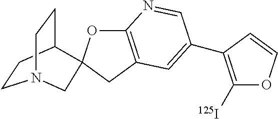 [SMILES: [125I]c1occc1-c1cnc2c(c1)CC1(CN3CCC1CC3)O2] | 0.26 | 0.6 | — | Pomper, et al. J. Nucl. Med. (2005) |
| 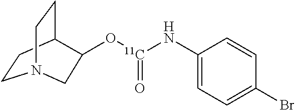 [SMILES: O=[11C](Nc1ccc(Br)cc1)OC1CN2CCC1CC2] | n/a | ~0.3 | — | Dolle, et al., J. Labelled Compd. Radiopharm. (2001) |
| 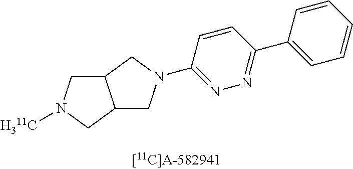 [SMILES: [11CH3]N1CC2CN(c3ccc(-c4ccccc4)nn3)CC2C1] | 10.8 | 0.2-0.5 | 0.3 | Toyohara, et al., PLoS One (2010) |
| 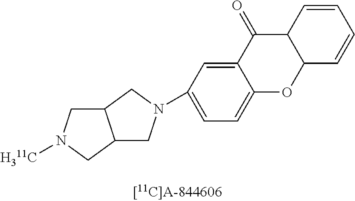 [SMILES: [11CH3]N1CC2CN(c3ccc4c(c3)C(=O)C3C=CC=CC3O4)CC2C1] | 11 | 0.6-0.7 | 0.5 | Toyohara, et al., PLoS One (2010) |
| 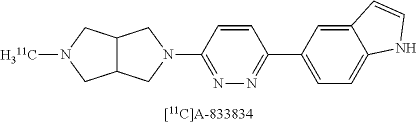 [SMILES: [11CH3]N1CC2CN(c3ccc(-c4ccc5[nH]ccc5c4)nn3)CC2C1] | 0.24, 1.53 | 0.5 | — | Horti, et al., Nucl. Med. Biol. (2013) |
| 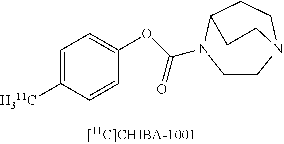 [SMILES: [11CH3]c1ccc(OC(=O)N2CCN3CCC2CC3)cc1] | 46, 120, 193 | ~0.6 | 0.6 | Hashimoto, et al., PLoS One (2008); Toyohara, et al., Ann. Nucl. Med. (2009); Tanibuchi, Y., et al., Brain Res. (2010); Ding, et al., Synapse (2012) |
| 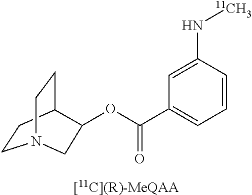 | 40.6 | 0.4 | 0.4 | Ogawa, et al., Nucl. Med. Biol. (2010) |
| 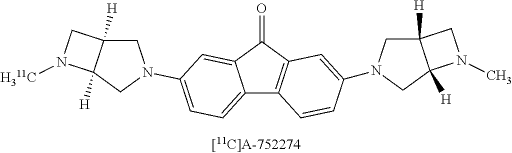 | 0.092 | low brain uptake | low brain uptake | Horti, et al., Nucl. Med. Biol. (2013) |
| 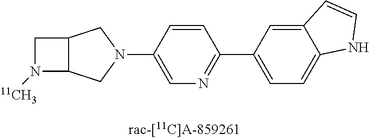 [SMILES: [11CH3]N1CC2CN(c3ccc(-c4ccc5[nH]ccc5c4)nc3)CC21] | 0.5-0.6 | 1.9 | — | Gao, et al., Bioorg. Med. Chem. (2012) |
| 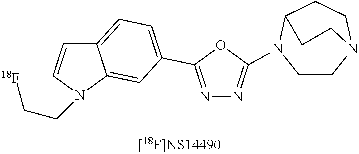 [SMILES: [18F]CCn1ccc2ccc(-c3nnc(N4CCN5CCC4CC5)o3)cc21] | 2.5 | low brain uptake | — | Rotering, et al., Bioorg. Med. Chem. (2013) |
| 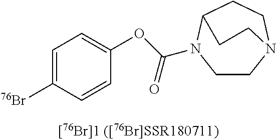 [SMILES: O=C(Oc1ccc([76Br])cc1)N1CCN2CCC1CC2] | 24.9 | — | 0.7 | Hashimoto, et al., PLoS One (2008) |
| 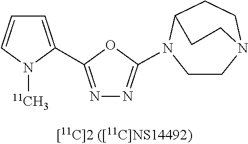 [SMILES: [11CH3]n1cccc1-c1nnc(N2CCN3CCC2CC3)o1] | 2.2 | — | ~1 | Ettrup, et al., J. Nucl. Med. (2011) |
| 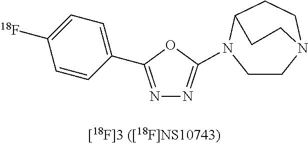 [SMILES: [18F]c1ccc(-c2nnc(N3CCN4CCC3CC4)o2)cc1] | 11.6 | 0.4 | 0.8 | Deuther-Conrad, et al., Eur. J. Nucl. Med. Mol. Imaging (2011) |
| 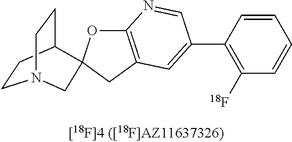 [SMILES: [18F]c1ccccc1-c1cnc2c(c1)CC1(CN3CCC1CC3)O2] | 0.2 | 0.8 | — | Ravert, et al., Nucl. Med. Biol. (2013); Maier, et al., Neuro- pharmacology (2011) |

^{a} The BP_{ND} values in the cortex were taken directly from the corresponding references or estimated as V_{T}/V_{ND} −1 or (cortex uptake/cerebellum uptake −1.
Innis, et al., J. Cereb. Blood Flow Metab. (2007); Tichauer, et al., Mol. Imaging Biol. (2011).

### SUMMARY

In one aspect, the presently disclosed subject matter provides a non-invasive method for imaging α7-nicotinic acetylcholine receptors (α7-nAChRs) in the brain of a subject, the method comprising administering to the subject an effective amount of a radiolabeled compound of Formula (I)

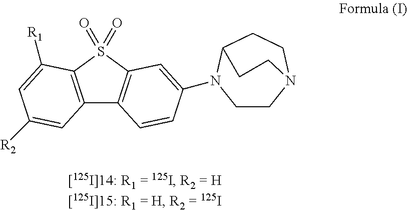

or a pharmaceutically acceptable salt, hydrate or prodrug thereof; and obtaining an image of the brain of the subject. In a particular aspect, the image is obtained by using single-photon emission computed tomography.

In another aspect, the presently disclosed subject matter provides a non-invasive method for quantifying one or more α7-nicotinic acetylcholine receptors (α7-nAChRs) in the brain of a subject, the method comprising: administering to the subject an effective amount of a radiolabeled compound of Formula (I)

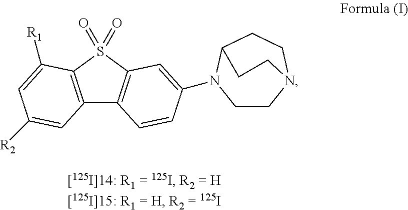

or a pharmaceutically acceptable salt, hydrate or prodrug thereof; allowing the radiolabeled compound to bind to the one or more α7-nAChR in the brain of the subject; obtaining an image of the brain of the subject showing the distribution of the radiolabeled compound; and deriving a standardized uptake value (SUV) from the image of the brain. In a particular aspect, the image is obtained by using single-photon emission computed tomography.

In another aspect, the presently disclosed subject matter provides a non-invasive method for imaging one or more α7-nicotinic acetylcholine receptors (α7-nAChRs) in the brain of a subject, the method comprising: administering to the subject an effective amount of [¹⁸F]-ASEM compound, or a pharmaceutically acceptable salt, hydrate or prodrug thereof; allowing the compound to bind to the one or more α7-nAChRs in the brain of the subject; and obtaining an image of the brain of the subject using positron emission tomography, wherein the binding is reversible.

In another aspect, the presently disclosed subject matter provides a non-invasive method for quantifying one or more α7-nicotinic acetylcholine receptors (α7-nAChRs) in the brain of a subject, the method comprising: administering to the subject an effective amount of [¹⁸F]-ASEM compound, or a pharmaceutically acceptable salt, hydrate or prodrug thereof; allowing the compound to bind to the one or more α7-nAChRs in the brain of the subject; obtaining a positron emission tomography (PET) image of the brain of the subject showing the distribution of the compound; and deriving a standardized uptake value (SUV) from the image of the brain.

In other aspects, the presently disclosed subject matter provides non-invasive method for diagnosing a disease or condition associated with α7-nAChRs in a subject in need thereof, the method comprising: administering to the subject a composition comprising an effective amount of a radiolabeled compound of Formula (I), (II) or (III),

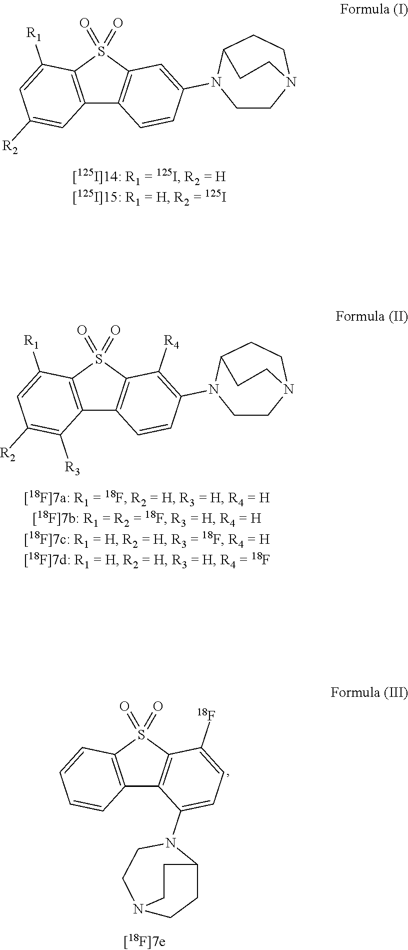

or a pharmaceutically acceptable salt, hydrate or prodrug thereof, allowing the radiolabeled compound to bind to the α7-nAChRs in the brain of the subject; and obtaining an imaging of the brain of the subject; wherein an alteration in the density of α7-nAChRs in the brain as compared to the brain of a subject without the disease condition is indicative that the subject has the disease, disorder, or condition associated with α7-nAChRs.

In certain aspects, the disease or condition is associated with α7-nAChRs is selected from the group consisting of schizophrenia, Alzheimer's disease, Parkinson's disease, anxiety, depression, attention deficit hyperactivity disorder (ADHD), multiple sclerosis, cancer, macrophage chemotaxis, inflammation, traumatic brain injury and drug addiction. In particular aspects, the radiolabeled compound readily enters the brain of the subject.

In further aspects, the radiolabeled compound is selected from the group consisting of

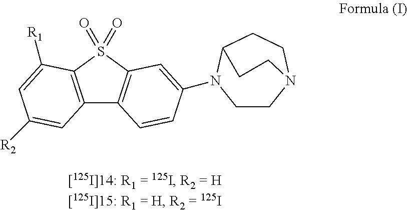

and the image is obtained by single-photon emission computed tomography.

In other aspects, the compound selectively binds to the α7-nAChRs relative toother nicotinic acetylcholine receptors.

In other aspects, the radiolabeled compound is selected from the group consisting of

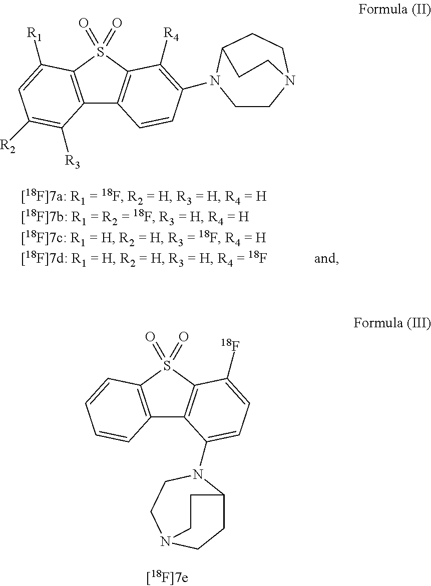

and the image is obtained by positron emission tomography.

In particular aspects, the radiolabeled compound is [18F]-ASEM.

In yet other aspects, the presently disclosed subject matter provides a method for preparing compounds of Formula (I)

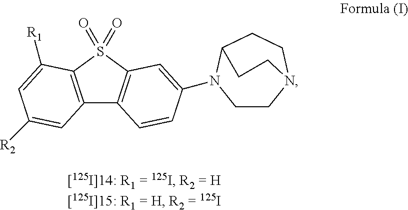

and compounds thereof.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows 3-(1,4-Diazabicyclo[3.2.2]nonan-4-yl)dibenzo[b,d]-thiophene 5,5-dioxide 5, an α7-nAChR antagonist with very high binding affinity, Schrimpf, et al., Bioorg. Med. Chem. Lett. (2012);

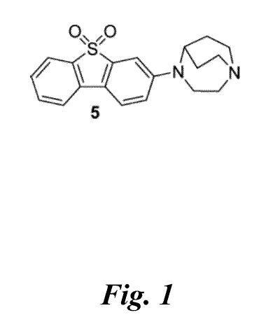

FIG. 2 shows the regional distribution of [¹⁸F]7a (left) and [¹⁸F]7c (right) in CD-1 mice. Data: mean % injected dose/g tissue ±SD (n=3). Abbreviations: Coll, superior and inferior colliculus; Hipp, hippocampus; FrCtx, frontal cortex; Rest, rest of brain; Th, thalamus; Str, striatum; CB, cerebellum;

FIG. 3 shows data from a self-blockade study of [¹⁸F]7a and [¹⁸F]7c in CD-1 mice. Left: Inhibition of [¹⁸F]7a (0.07 mCi, specific radioactivity of 9200 mCi/μmol, iv) accumulation by intravenous co-injection with 7a (0 mg/kg (white) and 0.3 mg/kg (black)) in the mouse brain regions 90 min after the injection: (*) P<0.01, significantly different from controls; (**) P=0.04, insignificantly different from controls (ANOVA). Right: Inhibition of [¹⁸F]7c (0.07 mCi, specific radioactivity of 12 000 mCi/μmol, iv) accumulation by intravenous co-injection with 7c (0 mg/kg (white) and 0.2 mg/kg (black)) in the mouse brain regions 90 min after the injection: (*) P<0.01, (**) P=0.015, significantly different from controls; (***) P=0.5, insignificantly different from controls (ANOVA). Data are the mean % injected dose/g tissue ±SD (n=3). Abbreviations: Coll, superior and inferior colliculus; Hipp, hippocampus; FrCtx, frontal cortex; Str, striatum; Rest, rest of brain, CB, cerebellum;

FIG. 4 shows blocking of [¹⁸F]7a and [¹⁸F]7c with α7-nAChR-selective ligands in CD-1 mice: (A) dose dependent blockade of [¹⁸F]7a (0.07 mCi, specific radioactivity of 7900 mCi/μmol, iv) accumulation by intravenous coinjection with 1 (doses 0.02, 0.2, 1, 3 mg/kg) in the mouse brain regions 90 min after the injection: (*) P<0.01, significantly different from controls (ANOVA); and (B) dose dependent blockade of [¹⁸F]7c (0.07 mCi, specific radioactivity of 11 000 mCi/μmol, iv) accumulation by intravenous co-injection with 5 (doses 0.001, 0.0045, 0.014 mg/kg) in the mouse brain regions 90 min after the injection: (*) P<0.01, significantly different from controls; (**) P=0.06, insignificantly different from control (ANOVA). Data are the mean % injected dose/g tissue ±SD (n=3). Abbreviations: Coll, superior and inferior colliculus; Hipp, hippocampus; Ctx, cortex; Str, striatum; Th, thalamus; Rest, rest of brain; CB, cerebellum;

FIG. 5 shows data from blockade of [¹⁸F]7a accumulation in CD-1 mouse brain regions by injection of cytisine (1 mg/kg, sc) and nicotine (5 mg/kg, sc) (both 90 min after the injection). Data are the mean % injected dose/g tissue ±SD (n=3). Abbreviations: Coll, superior and inferior colliculus; Hipp, hippocampus; Ctx, cortex; CB, cerebellum; Rest, rest of brain. The effect of cytisine was insignificant in all regions studied (P>0.05, asterisk is not shown). The difference between control and nicotine was significant ((*) P<0.01) in all regions except CB ((**) P=0.9) (ANOVA). The study demonstrates that [¹⁸F]7a does not bind in vivo at the main cerebral α4β2-nAChR subtype and it is suitable for nicotine blockade studies;

FIG. 6 shows the effect of various CNS drugs (Table 5) on accumulation of [¹⁸F]7a in CD-1 mouse brain regions 90 min after injection of tracer expressed as % ID/g tissue. Abbreviations: Coll, superior and inferior colliculus; Hipp, hippocampus; Ctx, cortex; CB, cerebellum; REST, rest of brain. Data are the mean±SD (n=3): (*) P<0.01, significantly different from controls. Columns that do not include the asterisk are insignificantly different from controls (P>0.05) (ANOVA, single-factor analysis). The graph demonstrates that unlike the positive control (1) all non-α7-nAChR CNS drugs do not have an effect on the cerebral uptake of [¹⁸F]7a and the radiotracer is α7-nAChR selective in vivo;

FIG. 7 shows the correlation of the BP_{ND} cortex (unitless) vs 1/Kᵢ (nM⁻¹) of α7 nAChR PET radioligands [¹¹C]2, [¹⁸F]3, [¹⁸F]4, [¹⁸F]7a, and [¹⁸F]7c (y=1.91x+0.52; R²=0.98). The BP_{ND} values are shown in Tables 1 and 3. The SD values are available for [¹⁸F]7a and [¹⁸F]7c only. All Kᵢ values were obtained in this study under the same binding assay conditions (Tables 2 and 3);

FIG. 8 shows the functional activity of unlabeled compound ASEM using whole-cell voltage clamp measurements in HEK293 cells expressing α7-nAChRs. [¹⁸F]ASEM inhibits the activation of acetylcholine-stimulated rat α7-nAChRs. Whole-cell voltage clamp current activated with 316 μM acetylcholine either before or during bath application of 1 nM [¹⁸F]ASEM was determined in HEK293 cells stably transfected with rat α7-nAChRs. Bath application of [¹⁸F]ASEM for 2 min before and during application of acetylcholine inhibited subsequent acetylcho-line-induced whole-cell current. This current was restored to 60% of baseline after 12 min of washing. ACh 5 acetylcholine;

FIG. 9A and FIG. 9B show the brain distribution of [¹⁸F]ASEM in Mutant DISC1 and Control Mice: (A) comparison of regional uptake of [¹⁸F]ASEM in control (black bars) and DISC1 (white bars) mice at 90 min after injection. There was significant reduction of [¹⁸F]ASEM in DISC1 in brain regions studied. Data are mean % ID/g tissue·body weight ±SD (n 5 6). *P 5 0.01 and **P, 0.01, significantly different from controls (ANOVA); and (B) Western blot. Expression of α7-nAChR protein in P21 cortex of mutant DISC1 (n 5 5) is significantly lower than in that of control mice (n 5 3). *P 5 0.035 (Student t test, t 5 2.7). Coll 5 superior and inferior colliculus; Ctx 5 cortex; Hipp 5 hippocampus;

FIG. 10 shows the baseline cerebral time-activity curves after bolus administration of [¹⁸F]-ASEM in 3 baboons. Graph demonstrates substantial heterogeneous brain uptake of [¹⁸F]-ASEM that matches distribution of α7-nAChR in nonhuman primates and reversible brain kinetics. Data are mean SUV (% SUV) ±SD (n=3). aCg=anterior cingulate cortex; CB=cerebellum; CC=corpus callosum; Hp=hippocampus; In=insula; Oc=occipital lobe; Pa=parietal lobe; Po=pons; Pu=putamen; Th=thalamus; Tp=temporal lobe;

FIG. 11A, FIG. 11B, and FIG. 11C show averaged transaxial % SUV PET images (10-90 min) of 18F-ASEM (upper) at levels showing: (A) putamen (Pun); (B) thalamus (Th/1); and (C) cortices such as frontal (Fr/1) and parietal (Pa/x), as shown on MR images (lower). SUV 5 standardized uptake value;

FIG. 12A and FIG. 12B show the regional V_{T} values in baseline and blockade experiments show: (A) Lassen plot for dose experiment of 5 mg/kg demonstrates that specific binding of [¹⁸F]-ASEM is blocked by α7-nAChR-selective ligand SSR180711. Data points showed linear appearance (ΔV_{T}=0.82·V_{T}−0.66; R²=0.979; V_{ND}=0.8 mL/mL) V_{ND} is given as x-intercept in plot; and (B) histogram of V_{T} values of 18F-ASEM (PRGA) in selected brain regions of 1 baboon at baseline and after administration of 2 different doses of SSR180711. Graph demonstrates that regional binding of ¹⁸F-ASEM is specific and high and mediated by α7-nAChR. aCg=anterior cingulate cortex; Cb=cerebellum; CC=corpus callosum; Hp=hippocampus; In=insula; Oc=occipital lobe; Pa=parietal lobe; Po=pons; Pu 5=putamen; Th=thalamus;

FIG. 13A, FIG. 13B, FIG. 13C, and FIG. 13D show Sagittal (top) and transaxial (middle and bottom) views of V_{T} images of [¹⁸F]ASEM in same baboon for baseline PET scan: (B) and after administration of 0.5 mg/kg (C) and 5 mg/kg (D) of SSR180711, a selective α7-nAChR partial agonist. MR images (A) indicate locations of selected brain structures including cingulate cortex (Cg), thalamus (Th), and caudate nucleus (CN), which are indicated by 1 in V_{T} images (D). V_{T} images were displayed using same minimum and maximum values for all scanning conditions. These data demonstrate dose-dependent blockade of [¹⁸F]ASEM in baboon brain and provide evidence that ¹⁸F-ASEM is specific and mediated by α7-nAChR. Images also suggest that there is no reference region devoid of α7-nAChRs;

FIG. 14A, FIG. 14B, FIG. 14C, and FIG. 14D show averaged (n=5) transaxial images of spatially normalized V_{T} map of [18F]ASEM and matching MRI in healthy control subjects. (a) Cerebellum (Cb) and medial temporal cortex (mdT) showed relatively low V_{T} values; (b) hippocampus (Hp) showed medium V_{T} values. (c) The insula (In), putamen (Pu), and thalamus (Th); (d) middle frontal (mFC), parietal (PC), and occipital (OC) cortices exhibited high V_{T} values in the human brain. Red dots on MRI images indicate outlines of cortical and subcortical structures;

FIG. 15 shows baseline PET/[¹⁸F]ASEM TAC [% SUV±SD (n=5)] in healthy human males. Pu, putamen; Pr, precuneus; Pa, parietal lobe; Th, thalamus; Fr, frontal lobe; Cg, cingulate; Oc, occipital; Tp, temporal lobe; Hp, hippocampus; CN, caudate nucleus; Cb, cerebellum; CC, corpus callosum. The distribution of [¹⁸F]-ASEM in the human brain regions is comparable with non-human primate and human post-mortem distribution of α7 The brain kinetics of [⁸F]-ASEM is reversible;

FIG. 16 shows a histogram (mean±SD bar) of regional values of distribution volume (V_{T}) for selected human brain regions. Regions are putamen (Pu), caudate nucleus (CN), ventral striatum (vS), global pallidus (GP), thalamus (Th), hippocampus (Hp), amygdala (Am), cingulate (Cg), frontal lobe (Fr), occipital lobe (0c), entorhinal area (ER), fusiform gyrus (Fs), parietal lobe (Pa), temporal lobe (Tp), parahippocampus (PH), paracentral (pC), post-central gyrus (PS), pre-central gyms (Pc), precuneus (Pr), insula (In), cerebellum (Cb), corpus callosum (CC);

FIG. 17A and FIG. 17B show [¹⁸F]ASEM Metabolite Analysis in Human Plasma: (A) time-profile (mean of five subjects with one SD bars) of parent fraction [¹⁸F]ASEM in plasma after the injection; and (B) total and metabolite-corrected plasma time-activity curves (TACs; mean of five subjects) expressed in SUV with an insert showing plots in the first 5 min. Coefficients of variation (SD over mean expressed in percentage) ranged from 21.1 and 27.2% (t910 min) for metabolite-corrected TACs; and

FIG. 18A, FIG. 18B, and FIG. 18C show the baseline versus blockade studies of [¹⁸F]ASEM with mouse-equivalent doses of clinical α7-nAChR drugs in CD1 mice. Data: % ID/g tissue±SD (n=4). The control mice were treated with vehicle saline. CB, cerebellum; Hipp, hippocampus; Ctx, cortex. Statistics for all three drugs: *PG0.01, blockade is significantly different from controls (ANOVA): (A) DMXB-A (GTS-21), dose escalation. A mouse-equivalent dose=25 mg/kg of the clinical dose (150 mg). Ninety-min post-[¹⁸F]ASEM injection; (B) EVP-6124, a mouse-equivalent dose (0.18 mg/kg) of the clinical dose (1 mg). Sixty-min post-[¹⁸F]-ASEM injection; and (C) varenicline, a mouse-equivalent dose (0.18 mg/kg) of the clinical dose (1 mg). Sixty-min post-[¹⁸F]-ASEM injection. The graph demonstrates that in vivo binding of [¹⁸F]-ASEM in the mouse brain regions enriched with α7-nAChR is significantly blocked by the α7-nAChR drugs DMXB-A, EVP-6124, and varenicline.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

The α7 nicotinic cholinergic receptor (α7-nAChR) is a key mediator of brain communication and has been implicated in a wide variety of central nervous system disorders. However, despite its importance, the physiological and pharmacological roles played by these receptors in the central nervous and peripheral system are still not fully understood. The lack of radioligands for quantitative emission tomography imaging of cerebral α7-nAChR receptors in man represents a gap that hampers non-invasive research of the α7-nAChR receptor system.

The presently disclosed subject matter discloses non-invasive methods for imaging, and quantifying the α7 nicotinic cholinergic receptors, as well as non-invasive methods for diagnosing a disease or condition associate with cerebral neuronal nicotinic cholinergic receptors. The presently disclosed subject matter also discloses a method for radiolabelling derivatives of dibenzothiophene and compounds provided thereof.

The presently disclosed subject matter describes the design, synthesis and in vitro and in vivo characterization in mice of a series of high α7-nAChR binding affinity compounds as potential probes for PET imaging of α7-nAChR receptor. In some embodiments, the presently disclosed subject matter provides a series of derivatives of 3-(1,4-diazabicyclo[3.2.2]nonan-4-yl)dibenzo[b,d]thiophene 5,5-dioxide. The presently disclosed compounds exhibit high binding affinities and selectivity for α7-nicotinic acetylcholine receptors (α7-nAChRs). For example, in some embodiments, the presently disclosed compounds exhibit a Kᵢ having a range between about 0.4 nM to about 20 nM. Particular embodiments of the presently disclosed compounds been synthesized for positron emission tomography (PET) imaging of α7-nAChRs. More particularly, two radiolabeled members of the series, [¹⁸F]7a (Kᵢ=0.4 nM) and [¹⁸F]7c (Kᵢ=1.3 nM) were synthesized. [¹⁸F]7a and [¹⁸F]7c readily entered the mouse brain and specifically labeled α7-nAChRs. The α7-nAChR selective ligand 1 (SSR180711) blocked the binding of [¹⁸F]7a in the mouse brain in a dose-dependent manner. The mouse blocking studies with non-α7-nAChR central nervous system drugs demonstrated that [¹⁸F]7a is highly α7-nAChR selective. In agreement with its binding affinity, the binding potential of [¹⁸F]7a (BP_{ND}=5.3-8.0) in control mice is superior to previous α7-nAChR PET radioligands. Thus, [¹⁸F]7a displays excellent imaging properties in mice and can potential for use as a PET radioligand for imaging of α7-nAChR in subjects.

### I. Non-Invasive Methods for Imaging Cerebral Neuronal Nicotinic Cholinergic Receptors in the Brain of a Subject

In some embodiments, the presently disclosed subject matter provides non-invasive methods for imaging one or more α7-nicotinic acetylcholine receptors (α7-nAChRs) in the brain of a subject.

Accordingly, in some embodiments, the presently disclosed subject matter provides a non-invasive method for imaging one or more α7-nicotinic acetylcholine receptors (α7-nAChRs) in the brain of a subject, the method comprising: administering to the subject an effective amount of a radiolabeled compound of Formula (I)

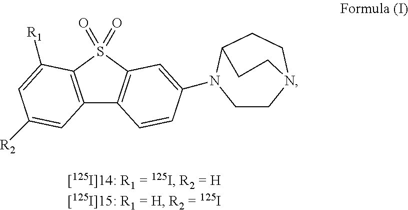

or a pharmaceutically acceptable salt, hydrate or prodrug thereof; allowing the radiolabeled compound to bind to the α7-nAChRs in the brain of the subject; and obtaining an image of the α7-nAChRs in the brain of the subject. In further embodiments, the image is obtained by using single-photon emission computed tomography. In still other embodiments, the compound selectively binds to the one or more α7-nAChRs relative to other nicotinic acetylcholine receptors in the brain. In yet others embodiments, the radiolabeled compound readily enters the brain of the subject. In other embodiments, the presently disclosed subject matter provides non-invasive method for imaging one or more α7-nicotinic acetylcholine receptors (α7-nAChRs) in the brain of a subject, the method comprising: administering to the subject an effective amount of [¹⁸F]-ASEM compound, or a pharmaceutically acceptable salt, hydrate or prodrug thereof; allowing the compound to bind to the one or more α7-nAChRs in the brain of the subject; and obtaining an image of the brain of the subject using positron emission tomography, wherein the binding is reversible. In other embodiments, the compound readily enters the brain of the subject. In still other embodiments, the specificity of the binding is at least about 80 percent. In further embodiments, the compound exhibits a percentage standardized uptake value of about 400 at 10 to 15 minutes. In yet further embodiments, the binding is reversible within approximately 90 minutes.

The term “non-invasive” as used herein refers to methods where no instruments are introduced into the body.

The term “administering” as used herein refers to contacting a α7-nAChR or portion thereof with a compound of Formula (I) or [¹⁸F]-ASEM compound. This term includes administration of the presently disclosed compounds to a subject in which the α7-nAChR or portion thereof is present, as well as introducing the presently disclosed compounds into a medium in which one or more α7-nAChRs or portion thereof is cultured.

By “selectively” is meant that the compounds of Formula (I) have a tendency to bind to a limited type of receptors, which in the presently disclosed subject matter are the α7-nicotinic acetylcholine receptors.

By “readily” is meant that the compounds of Formula (I) or [¹⁸F]-ASEM compound enter directly the brain of the subject after administration.

[¹⁸F]7a and [¹⁸F]-ASEM are used interchangeably but understood to refer to the compound having the following chemical structure:

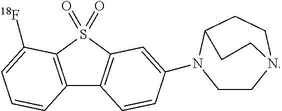 [SMILES: O=S1(=O)c2cc(N3CCN4CCC3CC4)ccc2-c2cccc([18F])c21]

Molecular imaging is the noninvasive visualization, characterization, and measurement of biological processes at the molecular and cellular levels in humans and other living systems. The present invention relates to compositions and methods for imaging, quantifying and diagnosing using positron emission tomography (PET) and single-photon emission computed tomography (SPECT). PET is the most advanced technique to map and quantify cerebral receptors and their occupancy by neurotransmitters and drugs in human subject. However, due to its lower cost compare to PET and its availability, SPECT is the most widely used technique to provide 3D informations.

The compounds used by the methods described herein are PET or SPECT radioligands suitable for quantitative PET or SPECT imaging and drug evaluation studies. For PET imaging, the compounds may be radiolabeled with radioactive isotopes, such as for example tritium (³H), fluorine-18 (¹⁸F), or carbon-14 (¹⁴C). The radiosiotope present on the radioligand emits a positron, which travels in tissue for a short distance during which time it loses kinetic energy, and then interact with an electron. The positron and electron are both annihilated, producing a pair of annihilation photons (gamma rays) moving in approximately opposite directions. These are detected by positron emission tomography (PET) using a suitable scanning device. The SPECT radioligands differ from PET radioligands in that they stay in the bloodstream rather than being absorbed by surrounding tissues, and therefore last longer in the subject. The compounds may be radiolabeled with radioactive isotopes, such as for example technetium-99m (⁹⁹Tc), iodine-125 (¹²⁵I) or xenon-133 (¹³³Xe). The radioisotope present on the radioligand emits gamma radiation that is directly measured using a suitable scanning device.

### II. Methods for Quantifying Cerebral Neuronal Nicotinic Cholinergic Receptors in the Brain of a Subject

In some embodiments, the presently disclosed subject matter provides non-invasive methods for quantifying one or more α7-nicotinic acetylcholine receptors (α7-nAChRs) in a subject.

Accordingly, in some embodiments, the presently disclosed subject matter provides a non-invasive method for quantifying one or more α7-nicotinic acetylcholine receptors (α7-nAChRs) in the brain of a subject, the method comprising: administering to the subject an effective amount of a radiolabeled compound of Formula (I), or a pharmaceutically acceptable salt, hydrate or prodrug thereof; allowing the radiolabeled compound to bind to the one or more α7-nAChRs in the brain of the subject; obtaining an image of the brain of the subject showing the distribution of the radiolabeled compound; and deriving a standardized uptake value (SUV) from the image of the brain. In other embodiments, the image is obtained by using single-photon emission computed tomography. In still other embodiments, the compound selectively binds to the one or more α7-nAChRs relative to other nicotinic acetylcholine receptors in the brain. In further embodiments, the radiolabeled compound readily enters the brain of the subject.

In some embodiments, the presently disclosed subject matter provides a non-invasive method for quantifying one or more α7-nicotinic acetylcholine receptors (α7-nAChRs) in a subject, the method comprising: administering to the subject an effective amount of [18F]-ASEM, or a pharmaceutically acceptable salt, hydrate or prodrug thereof; obtaining a PET image of the brain of the subject showing the regional brain distribution of the compound; and deriving a standardized uptake value (SUV) from the image of the brain. In other embodiments, the compound readily enters the brain of the subject. In still other embodiments the specificity of the binding is at least about 80 percent. In further embodiments, the compound exhibits a percentage standardized uptake value of about 400 at 10 to 15 minutes. In yet further embodiments, the binding is reversible within approximately 90 minutes.

III. Method for Diagnosing a Disease or Condition Associated with Cerebral Neuronal Nicotinic Cholinergic Receptors

In some embodiments, the presently disclosed subject matter provides a non-invasive method for diagnosing a disease or condition associated with α7-nAChRs in a subject in need thereof, the method comprising: administering to the subject a composition comprising an effective amount of a radiolabeled compound of Formula (I), (II) or (III):

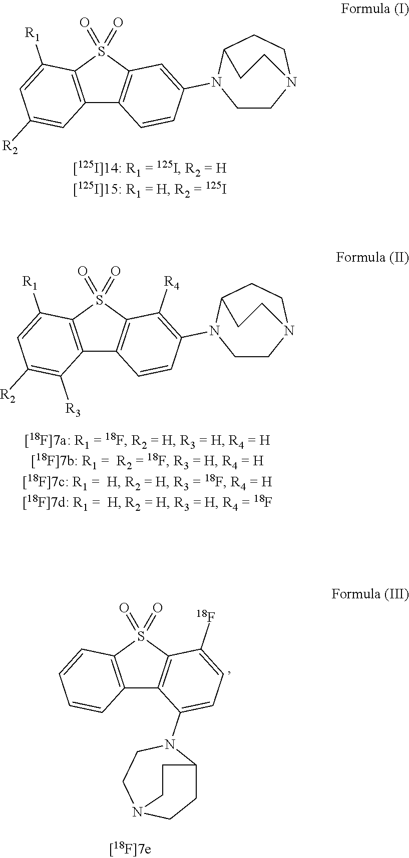

or a pharmaceutically acceptable salt, hydrate or prodrug thereof, allowing the radiolabeled compound to bind to the α7-nAChRs in the brain of the subject; and obtaining an imaging of the brain of the subject; wherein an alteration in the density of α7-nAChRs in the brain as compared to the brain of a subject without the disease condition is indicative that the subject has the disease, disorder, or condition associated with α7-nAChRs. In other embodiments, the disease or condition associated with α7-nAChRs is selected from the group consisting of schizophrenia, Alzheimer's disease, Parkinson's disease, anxiety, depression, attention deficit hyperactivity disorder (ADHD), multiple sclerosis, cancer, macrophage chemotaxis, inflammation, traumatic brain injury and drug addiction.

In some embodiments, the radiolabeled compound readily enters the brain of the subject. In other embodiments, the radiolabeled compound is selected from the group consisting of

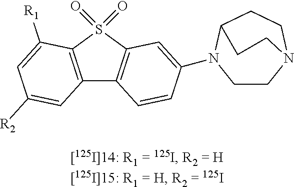

and the image is obtained by single-photon emission computed tomography. In still other embodiments, the compound selectively binds to the α7-nAChRs relative to other nicotinic acetylcholine receptors.

In some embodiments, the radiolabeled compound is selected from the group consisting of

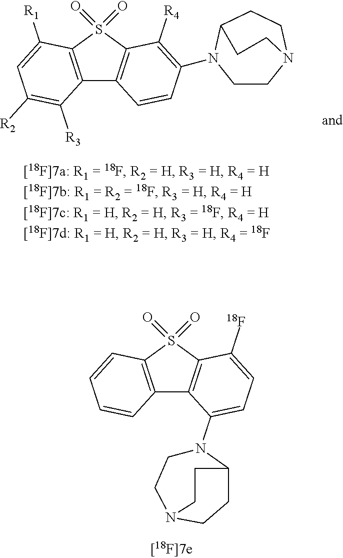

and the image is obtained by positron emission tomography. In other embodiments, the radiolabeled compound is [18F]-ASEM. In still other embodiments, the specificity of the binding is at least 80 percent. In further embodiments, the radiolabeled compound exhibits a percentage of standardized uptake value of about 400 at 10 to 15 minutes. In still further embodiments, the binding is reversible within approximately 90 minutes.

As used herein, the term “diagnosis” refers to a predictive process in which the presence, absence, severity or course of treatment of a disease, disorder or other medical condition is assessed. For purposes herein, diagnosis also includes predictive processes for determining the outcome resulting from a treatment. Likewise, the term “diagnosing,” refers to the determination of whether a sample specimen exhibits one or more characteristics of a condition or disease. The term “diagnosing” includes establishing the presence or absence of, for example, a reagent bound target molecule, or otherwise determining one or more characteristics of a condition or disease, including type, grade, stage, or similar conditions. As used herein, the term “diagnosing” can include distinguishing one form of a disease from another. The term “diagnosing” encompasses the initial diagnosis or detection, prognosis, and monitoring of a condition or disease. The term “prognosis” and derivations thereof, refers to the determination or prediction of the course of a disease or condition. The course of a disease or condition can be determined, for example, based on life expectancy or quality of life. “Prognosis” includes the determination of the time course of a disease or condition, with or without a treatment or treatments. In the instance where treatment(s) are contemplated, the prognosis includes determining the efficacy of a treatment for a disease or condition. The term “monitoring,” such as in “monitoring the course of a disease or condition,” refers to the ongoing diagnosis of samples obtained from a subject having or suspected of having a disease or condition.

As used herein, the term “disease or disorder” in general refers to any condition that would need a diagnosis with a compound against one of the identified targets, or pathways, including any disease, disorder, or condition that can be diagnosed by an effective amount of a compound against one of the identified targets, or pathways, or a pharmaceutically acceptable salt thereof.

### IV. Method for Radiolabelling Derivatives of Dibenzothiophene and Compounds Provided Thereof

A. Method for Radiolabelling Derivatives of Dibenzothiophene

In some embodiments, the presently disclosed subject matter provides a method for radiolabeling a compound of Formula (I):

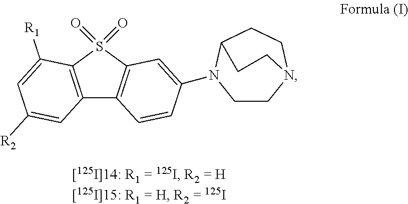

the method comprising:

(a) contacting a solution of a compound of Formula (IV)

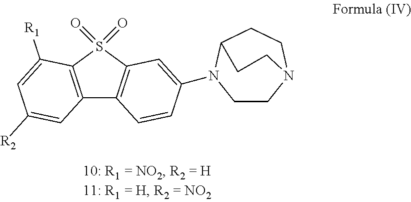

in a solvent with Na ¹²⁵I to form a mixture;

(b) adding an acid to the mixture;

(c) heating the mixture;

(d) cooling the mixture;

(e) diluting the mixture in an appropriate solvent;

(f) applying the diluted mixture to a reverse phase HPLC column;

(g) collecting the radioactive peak;

(h) transferring the radioactive peak to a solid phase extraction (SPE) cartridge;

(i) eluting the product through a filter; and

(j) adding saline and a solution of sodium bicarbonate through the filter to form Formula (I).

B. Radiolabeled Derivatives of Dibenzothiophene

In some embodiment, the presently disclosed subject matter provides a compound of Formula (I)

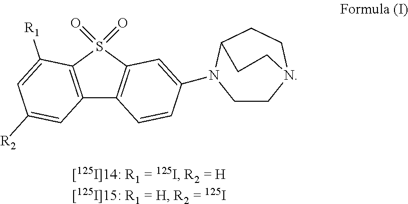

Without wishing to be bound to any one particular theory, it is believed that the presently disclosed compounds can modulate: (i) the activity or expression of a target protein in the neuron or portion thereof; (ii) a process in the neuron or portion thereof; or (iii) a biological pathway associated with a α7-nAChRs-related disease, disorder, or condition. In particular embodiments, the presently disclosed compounds inhibit one or more α7-nAChRs involved in a biological pathway associated with a disease, disorder, or condition.

As used herein, the term “inhibit” or “inhibits” means to decrease, suppress, attenuate, diminish, arrest, or stabilize the development or progression of a disease, disorder, or condition, or the activity of a biological pathway, e.g., by at least 10%, 20%, 30%, 40%, 50%, 60%, 70%, 80%, 90%, 95%, 98%, 99%, or even 100% compared to an untreated control subject, cell, or biological pathway. By the term “decrease” is meant to inhibit, suppress, attenuate, diminish, arrest, or stabilize a symptom of a particular disease, disorder, or condition. It will be appreciated that, although not precluded, treating a disease, disorder or condition does not require that the disease, disorder, condition or symptoms associated therewith be completely eliminated.

Accordingly, in some embodiments, a compound of Formula (I), (II) or (III) can be used to treat or prevent a disease, disorder, or condition. As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. The terms “treat,” “treating,” “treatment,” and the like, as used herein can refer to curative therapy, prophylactic therapy, and preventative therapy. The treatment, administration, or therapy can be consecutive or intermittent. Consecutive treatment, administration, or therapy refers to treatment on at least a daily basis without interruption in treatment by one or more days. Intermittent treatment or administration, or treatment or administration in an intermittent fashion, refers to treatment that is not consecutive, but rather cyclic in nature. Treatment according to the presently disclosed methods can result in complete relief or cure from a disease, disorder, or condition, or partial amelioration of one or more symptoms of the disease, disease, or condition, and can be temporary or permanent. The term “treatment” also is intended to encompass prophylaxis, therapy and cure.

As used herein, the terms “prevent,” “preventing,” “prevention,” “prophylactic treatment” and the like refer to reducing the probability of developing a disease, disorder, or condition in a subject, who does not have, but is at risk of or susceptible to developing a disease, disorder, or condition. Thus, in some embodiments, an agent can be administered prophylactically to prevent the onset of a disease, disorder, or condition, or to prevent the recurrence of a disease, disorder, or condition.

By “agent” is meant a compound of Formula (I), (II) or (III) compounds or another agent administered in combination with a compound of Formula (I), (II) or (III). More generally, the term “therapeutic agent” means a substance that has the potential of affecting the function of an organism. Such an agent may be, for example, a naturally occurring, semi-synthetic, or synthetic agent. For example, the therapeutic agent may be a drug that targets a specific function of an organism. A therapeutic agent also may be a nutrient. A therapeutic agent may decrease, suppress, attenuate, diminish, arrest, or stabilize the development or progression of disease, disorder, or condition in a host organism.

As used herein the term “disease or condition associated with α7-nAChRs” in general refers to any condition that would benefit from treatment with a compound of Formula (I), (II) or (III), including any disease or condition that can be treated by an effective amount of a compound of Formula (I), (II) or (III), or a pharmaceutically acceptable salt thereof. Such diseases or conditions include, but are not limited to, schizophrenia, Alzheimer's disease, Parkinson's disease, anxiety, depression, attention deficit hyperactivity disorder (ADHD), multiple sclerosis, cancer, macrophage chemotaxis, inflammation, traumatic brain injury and drug addiction.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments).

In any of the above-described methods, the administering of a compound of Formula (I), (II) or (III), can result in at least about a 10%, 15%, 20%, 25%, 30%, 35%, 40%, 45%, 50%, 55%, 60%, 65%, 70%, 75%, 80%, 85%, 90%, 95%, or even 100% decrease in one or more (e.g., 1, 2, 3, 4, 5, 6, 7, 8, 9, or 10) symptoms of a disease, disorder, or condition compared to a subject that is not administered the one or more of the agents described herein.

In any of the above-described methods, the administering of a compound of Formula (I) results in at least about a 10%, 15%, 20%, 25%, 30%, 35%, 40%, 45%, 50%, 55%, 60%, 65%, 70%, 75%, 80%, 85%, 90%, 95%, or even 100% decrease in the likelihood of developing a disease, disorder, or condition compared to a control population of subjects that are not administered a compound of Formula (I), (II) or (III).

The above-listed terms also include in vitro and ex vivo methods. For example, in certain embodiments, the presently disclosed methods are applicable to cell culture techniques wherein it is desirable to prevent neuronal cell death or loss of neuronal function.

C. Pharmaceutical Compositions

The presently disclosed pharmaceutical compositions and formulations include pharmaceutical compositions of compounds of Formula (I), (II) or (III), alone or in combination with one or more additional therapeutic agents, in admixture with a physiologically compatible carrier, which can be administered to a subject, for example, a human subject, for therapeutic or prophylactic treatment. As used herein, “physiologically compatible carrier” refers to a physiologically acceptable diluent including, but not limited to water, phosphate buffered saline, or saline, and, in some embodiments, can include an adjuvant. Acceptable carriers, excipients, or stabilizers are nontoxic to recipients at the dosages and concentrations employed, and can include buffers such as phosphate, citrate, and other organic acids; antioxidants including ascorbic acid, BHA, and BHT; low molecular weight (less than about 10 residues) polypeptides; proteins, such as serum albumin, gelatin or immunoglobulins; hydrophilic polymers, such as polyvinylpyrrolidone, amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counter-ions such as sodium; and/or nonionic surfactants such as Tween, Pluronics, or PEG. Adjuvants suitable for use with the presently disclosed compositions include adjuvants known in the art including, but not limited to, incomplete Freund's adjuvant, aluminum phosphate, aluminum hydroxide, and alum.

Compositions to be used for in vivo administration must be sterile, which can be achieved by filtration through sterile filtration membranes, prior to or following lyophilization and reconstitution. Therapeutic compositions may be placed into a container having a sterile access port, for example, an intravenous solution bag or vial having a stopper pierceable by a hypodermic injection needle.

One of skill in the art will recognize that the pharmaceutical compositions include the pharmaceutically acceptable salts of the compounds described above. The term “pharmaceutically acceptable salts” is meant to include salts of active compounds, which are prepared with relatively nontoxic acids or bases, depending on the particular substituent moieties found on the compounds described herein.

When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include alkali or alkaline earth metal salts including, but not limited to, sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids including, but not limited to, hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids, such as acetic (acetates), propionic (propionates), isobutyric (isobutyrates), maleic (maleates), malonic, benzoic (benzoates), succinic (succinates), suberic, fumaric (fumarates), lactic (lactates), mandelic (mandelates), phthalic (phthalates), benzenesulfonic (benzosulfonates), p-tolylsulfonic, citric (citrates), tartaric (tartrates, e.g., (+)-tartrates, (−)-tartrates or mixtures thereof including racemic mixtures), methanesulfonic, and the like. Other pharmaceutically acceptable salts, include, but are not limited to, besylate, bicarbonate, bitartrate, bromide, calcium edetate, carnsylate, carbonate, edetate, edisylate, estolate, esylate, gluceptate, gluconate, glutamate, glycollylarsanilate, hexylresorcinate, hydrabamine, hydroxynaphthoate, iodide, isethionate, lactobionate, malate, mesylate, mucate, napsylate, nitrate, pamoate (embonate), pantothenate, phosphate/diphosphate, polygalacturonate, salicylate, stearate, subacetate, sulfate, tannate, and teoclate, also are included.

Also included are salts of amino acids, such as arginate and the like, and salts of organic acids, such as, glucuronic or galactunoric acids, and the like. See, for example, Berge et al, “Pharmaceutical Salts”, Journal of Pharmaceutical Science, 1977, 66, 1-19. Some compounds of the present disclosure can contain both basic and acidic functionalities, which allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties. For example, salts tend to be more soluble in aqueous or other protonic solvents than are the corresponding free base forms.

In particular embodiments, the pharmaceutically acceptable salt of a compound of Formula (I) is selected from the group consisting of HCl, a sulfonate, a sulfate, phosphate, a malonate, a succinate, a fumarate, a maleate, a tartrate, a 3-sulfopropanoic acid salt, and a citrate. Suitable salts of the presently disclosed compounds are disclosed in International PCT Patent Application Publication No. WO2004/000833 to Charrier et al., published Dec. 31, 2003, which is incorporated herein by reference in its entirety.

Certain compounds of the present disclosure can exist in unsolvated forms, as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds that can be in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

D. Combination Therapies

In certain embodiments, presently disclosed subject matter also includes combination therapies. Depending on the particular disease, disorder, or condition to be treated or prevented, additional therapeutic agents, which are normally administered to treat or prevent that condition, may be administered in combination with the compounds of this disclosure. These additional agents may be administered separately, as part of a multiple dosage regimen, from the composition comprising a compound of Formula (I), (II) or (III). Alternatively, these agents may be part of a single dosage form, mixed together with the compound of Formula (I), (II) or (III), in a single composition.

By “in combination with” is meant the administration of a compound of Formula (I), (II) or (III), with one or more therapeutic agents either simultaneously, sequentially, or a combination thereof. Therefore, a cell or a subject administered a combination of a compound of Formula (I), (II) or (III), can receive a compound of Formula (I), (II) or (III), and one or more therapeutic agents at the same time (i.e., simultaneously) or at different times (i.e., sequentially, in either order, on the same day or on different days), so long as the effect of the combination of both agents is achieved in the cell or the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound of Formula (I), (II) or (III), and one or more therapeutic agents are administered simultaneously, they can be administered to the cell or administered to the subject as separate pharmaceutical compositions, each comprising either a compound of Formula (I), (II) or (III), or one or more therapeutic agents, or they can contact the cell as a single composition or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered compound is not diminished by the sequential, simultaneous or separate administration of the subsequent compound(s).

as two, three, four, five, six or more sub-doses administered separately at appropriate intervals throughout the day, optionally, in unit dosage forms.

E. Kits or Pharmaceutical Systems

The presently disclosed compounds and compositions can be assembled into kits or pharmaceutical systems for use in treating or preventing neurodegenerative diseases, disorders, or conditions. In some embodiments, the presently disclosed kits or pharmaceutical systems include a compound of Formula (I), (II) or (III), or pharmaceutically acceptable salts thereof. In particular embodiments, the compounds of Formula (I), (II) or (III), or a pharmaceutically acceptable salt thereof, are in unit dosage form. In further embodiments, the compound of Formula (I), (II) or (III), or a pharmaceutically acceptable salt, can be present together with a pharmaceutically acceptable solvent, carrier, excipient, or the like, as described herein.

In some embodiments, the presently disclosed kits comprise one or more containers, including, but not limited to a vial, tube, ampule, bottle and the like, for containing the compound. The one or more containers also can be carried within a suitable carrier, such as a box, carton, tube or the like. Such containers can be made of plastic, glass, laminated paper, metal foil, or other materials suitable for holding medicaments.

In some embodiments, the container can hold a composition that is by itself or when combined with another composition effective for treating or preventing the condition and may have a sterile access port (for example the container may be an intravenous solution bag or a vial having a stopper pierceable by a hypodermic injection needle). Alternatively, or additionally, the article of manufacture may further include a second (or third) container including a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The presently disclosed kits or pharmaceutical systems also can include associated instructions for using the compounds for treating or preventing a neurodegenerative disease, disorder, or condition. In some embodiments, the instructions include one or more of the following: a description of the active compound; a dosage schedule and administration for treating or preventing a neurodegenerative disease, disorder, or condition; precautions; warnings; indications;

counter-indications; overdosage information; adverse reactions; animal pharmacology; clinical studies; and references. The instructions can be printed directly on a container (when present), as a label applied to the container, as a separate sheet, pamphlet, card, or folder supplied in or with the container.

F. Chemical Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of Formula (I), (II), (III) or (IV) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Description of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁-C₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “heteroalkyl,” by itself or in combination with another term, means, unless otherwise stated, a stable straight or branched chain, or cyclic hydrocarbon group, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂₅—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₁₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₁₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

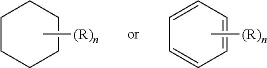

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

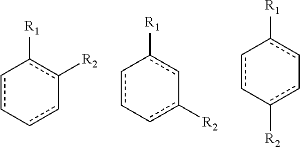

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond. Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R″″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds, which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

All reagents were used directly as obtained commercially unless otherwise noted. Reaction progress was monitored by TLC using silica gel 60 F254 (0.040-0.063 mm) with detection by UV. All moisture sensitive reactions were performed under an argon atmosphere using oven-dried glassware and anhydrous solvents. Column flash chromatography was carried out using E. Merck silica gel 60F (230-400 mesh). Analytical thin-layer chromatography (TLC) was performed on aluminum sheets coated with silica gel 60 F254 (0.25 mm thickness, E. Merck, Darmstadt, Germany). Melting points were determined with a Fisher-Johns apparatus and were not corrected. 1H NMR spectra were recorded with a Bruker-400 NMR spectrometer at nominal resonance frequencies of 400 MHz in CDCl₃ or DMSO-d₆ (referenced to internal Me₄Si at δH 0 ppm). The chemical shifts (δ) were expressed in parts per million (ppm). First order J values were given in hertz. Splitting patterns are described as singlet (s), doublet (d), triplet (t), quartet (q), and broad (br). High resolution mass spectra were recorded utilizing electrospray ionization (ESI) at the University of Notre Dame Mass Spectrometry facility. All compounds that were tested in the biological assays were analyzed by combustion analysis (CHN) to confirm the purity of >95%. Elemental analyses were determined by Galbraith Laboratories, Inc. (Knoxville, Tenn.). The HPLC system consisted of two Waters model 600 pumps, two Rheodyne model 7126 injectors, an in-line Waters model 441 UV detector (254 nm), and a single sodium iodide crystal flow radioactivity detector. All HPLC chromatograms were recorded with Varian Galaxy software (version 1.8). The analytical and semipreparative chromatographies were performed using Waters XBridge C-18 10 μm columns (analytical 4.6 mm×250 mm and preparative 10 mm×250 mm) A dose calibrator (Capintec 15R) was used for all radioactivity measurements. Radiofluorination was performed with a modified GE MicroLab radiochemistry box.

The Animal Care and Use Committee of the Johns Hopkins Medical Institutions approved all experimental animal protocols.

Healthy young volunteers aged 27-49 years (mean age 43.6±4.17 SEM, n=5) were recruited from the Baltimore Metropolitan area. All subjects received informed consent approved by the Johns Hopkins School of Medicine Investigational Review Board. Imaging studies were preceded by appropriate toxicology and safety studies including radiation dosimetry carried out in mouse organ biodistribution studies resulting in an FDA-approved IND. Subjects were screened for the absence of significant neuropsychiatric and medical disorders (the inclusion criteria included healthy volunteers between 18 and 65 years old and BMI between 18 and 30 kg/m₂ inclusive and the exclusion criteria included smoking, drug or alcohol dependence, and any use of acetylcholinesterase inhibitors or prior psychotropic drugs. Screening procedures included a complete medical and medication history, demography, physical exam [including height, weight, and body mass index (BMI)], vital signs, 12-lead electrocardiogram (ECG), and laboratory safety tests. All subjects agreed to receive a radial arterial line for blood sampling.

### Example 1

### Chemistry

### Synthesis of α7-nAChR Ligands

The fluoro derivatives 7a-e of 3-(1,4-diazabicyclo[3.2.2]nonan-4-yl)-dibenzo[b,d]thiophene 5,5-dioxide 5 were synthesized via the Buchwald-Hartwig cross-coupling reaction between the respective fluorobromo compounds 6a-e with 1,4-diazabicyclo[3.2.2]nonane (Scheme 1).

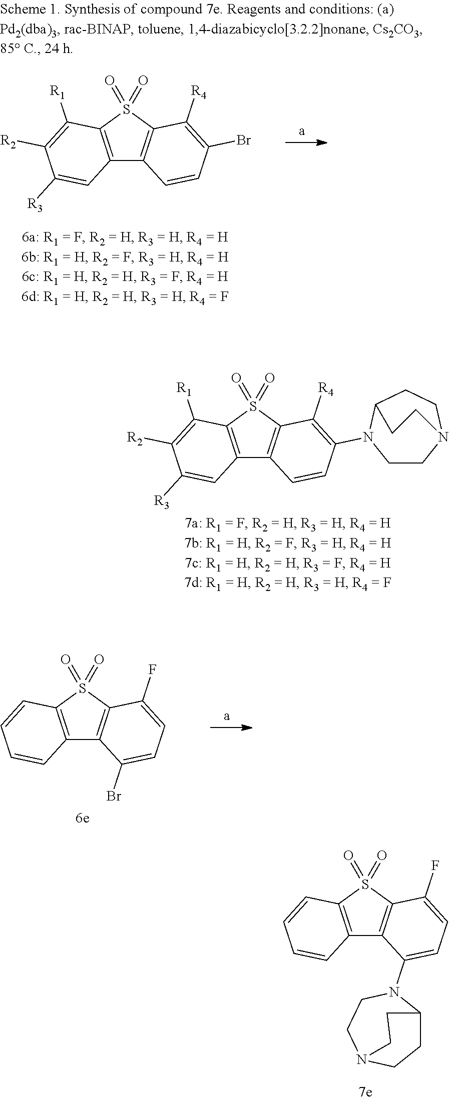

The nitro derivatives of (1,4-diazabicyclo[3.2.2]nonan-4-yl)dibenzo[b,d]thiophene 5,5-dioxide 10 and 11 were synthesized similarly starting with respective nitrobromodibenzothiophene derivatives 8 and 9 (Scheme 2). Reduction of nitro groups in 10 and 11 with iron powder gave corresponding anilines 12 and 13 in high yield. Diazotization-iodination of 12 and 13 yielded corresponding iodides 14 and 15 (Scheme 2).

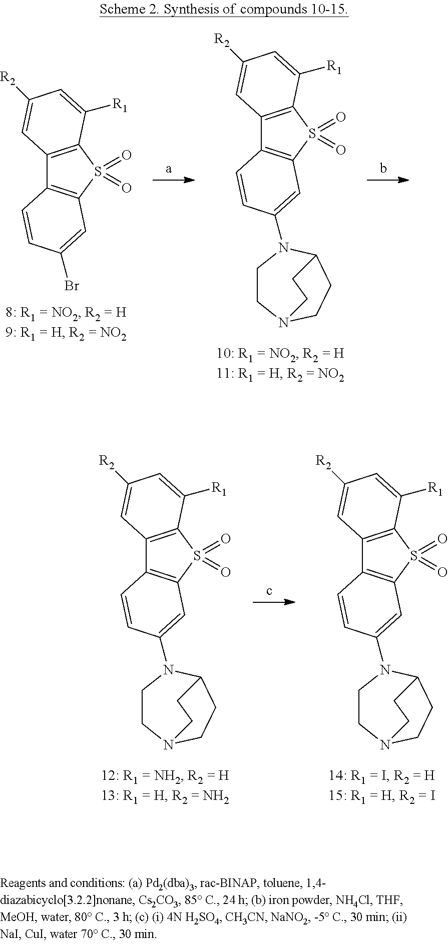

### Synthesis of Intermediate Compounds

The synthesis of intermediate fluorobromide 6a was performed in four steps (Scheme 3). Coupling of commercially available 4-bromo-2-fluoronitrobenzene 16 and 2-fluorothiophenol 17 gave nitrodiaryl thioether 18 that was reduced to aniline 19. Aniline 19 was treated with sodium nitrite at 0° C. in the presence of hydrochloric acid and sodium tetrafluoroborate to yield a corresponding diazonium tetrafluoroborate derivative (not shown). The intramolecular deazotization/cyclization of the diazonium salt in the presence of copper(I) oxide and 0.1 N sulfuric acid afforded fluorobromodibenzothiophene derivative 20, which in turn was oxidized with hydrogen peroxide to 6a in high yield (Scheme 3).

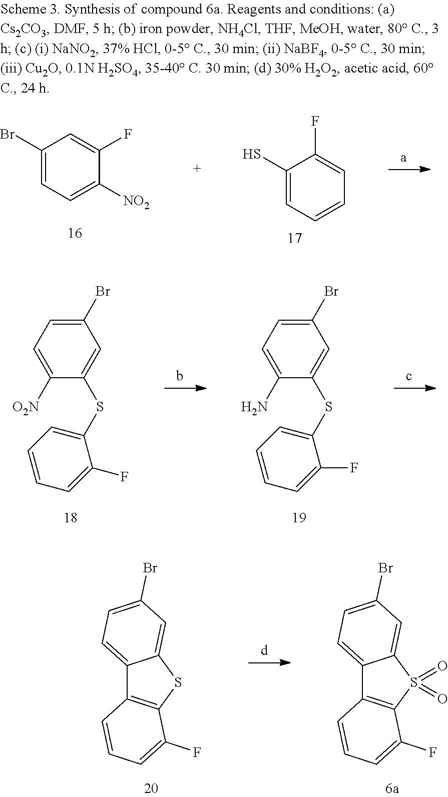 [SMILES: O=[N+]([O-])c1ccc(Br)cc1F ; Fc1ccccc1S ; O=[N+]([O-])c1ccc(Br)cc1Sc1ccccc1F ; Nc1ccc(Br)cc1Sc1ccccc1F ; Fc1cccc2c1sc1cc(Br)ccc12 ; O=S1(=O)c2cc(Br)ccc2-c2cccc(F)c21]

The fluorobromo isomers 6b and 6c were synthesized in four steps via the commercially available dibenzo[b,d]thiophene 5,5-dioxide 21 and 2-nitrodibenzo[b,d]thiophene 22, respectively (Scheme 4).

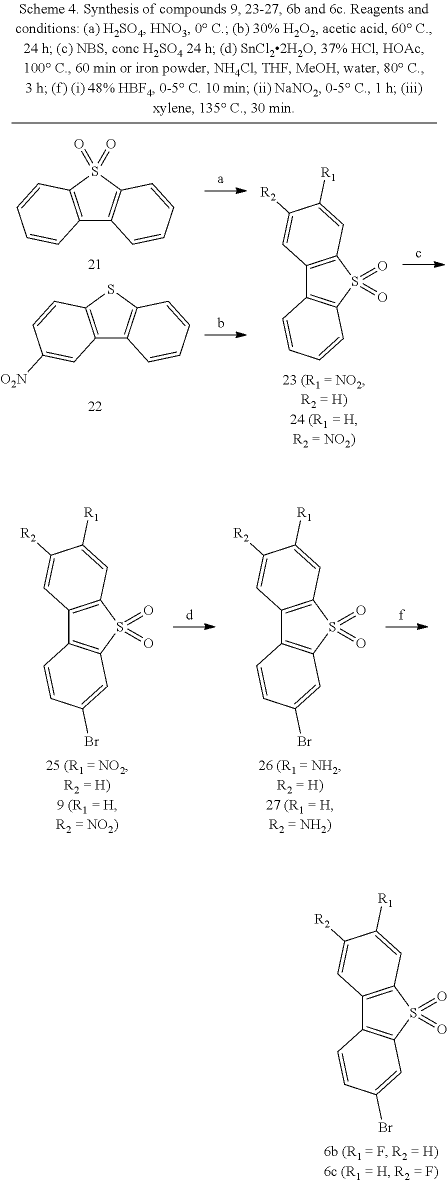

In brief, nitration of compound 21 and oxidation of compound 22 gave compounds 23 and 24, respectively. Bromination of compounds 23 and 24 provided monobromo derivatives 25 and 9 that sequentially were reduced to anilines 26 and 27, respectively, in high yields. The anilines 26 and 27 were converted to fluorides 6b and 6c in moderate yields by the Schiemann reaction via the corresponding intermediate diazonium fluoroborates (structures not shown). The diazonium salts precipitated in the reaction mixture and were isolated by filtration in high yields. The brominated isomers 6d and 6e were prepared by bromination of 4-fluorodibenzo[b,d]thiophene 5,5-dioxide 29 starting with 4-fluorodibenzo[b,d]thiophene 28 (Scheme 5). Nag and Jenks, J. Org. Chem. (2005).

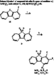

Oxidation of 28 with hydrogen peroxide gave dioxide 29 in nearly quantitative yield. Bromination of 29 with 1 equiv of NBS in H₂SO₄ afforded two isomeric bromides: 6d as the main product in 24% yield and 6e as a minor product in 13% yield. A substantial amount of compound 29 (about 50%) was recovered from the reaction mixture. Isomers 6d and 6e were readily separated by silica gel chromatography.

3-Bromo-6-nitrodibenzo[b,d]thiophene 5,5-dioxide 8 was synthesized in two steps: (1) oxidation of 4-nitrodibenzo[b,d]-thiophene 30, Manna, et al., Org. Lett. (2012), gave 4-nitrodibenzo[b,d]thiophene 5,5-dioxide 31 in 90% yield; (2) bromination of compound 31 provided compound 8 as the only product in 77% yield (Scheme 6).

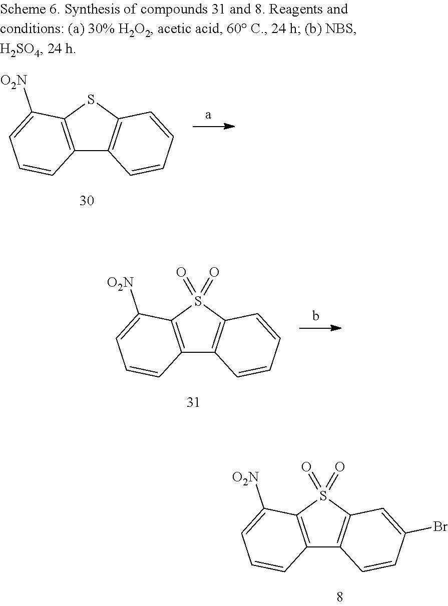 [SMILES: O=[N+]([O-])c1cccc2c1sc1ccccc12 ; O=[N+]([O-])c1cccc2c1S(=O)(=O)c1ccccc1-2 ; O=[N+]([O-])c1cccc2c1S(=O)(=O)c1cc(Br)ccc1-2]

### In Vitro Inhibition Binding Assay

The results of the α7-nAChR in vitro inhibition binding assays for compounds 7a-e, 10, 11, 14, and 15 are shown in Table 2. To determine α7-nAChR selectivity of new compounds vs other nAChR subtypes, binding assays for the main cerebral heteromeric nAChR subtypes (α2β2, α2β4, α3β2, α3β4, α4β2, and α4β4) also were performed (Table 2). In addition, because α7-nAChR shares 30% homology with the 5-HT3 receptor and first generation α7-nAChR radioligands exhibited low α7-nAChR/5-HT3 selectivity, Ravert, et al., Nucl. Med. Biol. (2013), the in vitro binding affinity at the 5-HT3 receptor also was determined for selected compounds of the presently disclosed series (Table 2).

TABLE 2
Inhibition of In Vitro Binding Affinities (Kᵢ, nM) of the New Series 7a-e, 10, 11, 14, and 15
toward α7-nAChR, Heteromeric nAChR Subtypes, and 5-HT3 heteromeric nAChR subtypeb selectivity.

| Compound | α7-nAChRa | α2β2 | α2β4 | α3β2 | α3β4 | α4β2 | α4β4 | 5-HT₃ c | α7/α4β2 | α7/5HT₃ |
|---|---|---|---|---|---|---|---|---|---|---|
| 7a | 0.37, | >10000 | 4000 | 1000 | 709 | 562 | 1000 | 230 | 1370 | 561 |
| | 0.45 | | | | | | | | | |
| 7b | 1.02, | ntd | ntd | ntd | ntd | ntd | ntd | ntd | | |
| | 1.37 | | | | | | | | | |
| 7c | 1.32, | 1000 | 8000 | 2000 | 5000 | 885 | 3000 | 505 | 663 | 378 |
| | 1.35 | | | | | | | | | |
| 7d | 1.83, | 292 | 838 | 678 | 3000 | 141 | 1000 | ntd | 66 | |
| | 2.45 | | | | | | | | | |
| 7e | 17.8, | >10000 | 562 | 2000 | 261 | 4000 | 251 | ntd | 210 | |
| | 20.3 | | | | | | | | | |
| 10 | 0.34, | ntd | ntd | ntd | ntd | ntd | ntd | ntd | | |
| | 0.35 | | | | | | | | | |
| 11 | 3.41, | ntd | ntd | ntd | ntd | ntd | ntd | ntd | | |
| | 6.21 | | | | | | | | | |
| 14 | 0.93, | ntd | ntd | ntd | ntd | ntd | ntd | ntd | | |
| | 1.93 | | | | | | | | | |
| 15 | 6.46, | 784 | 6000 | 1000 | 9000 | 477 | 5000 | ntd | 63 | |
| | 8.77 | | | | | | | | | |

aRat cortical membranes, radiotracer [¹²⁵I]α-bungarotoxin (0.1 nM). KD = 0.7 nM.
bInhibition in vitro binding assay of all heteromeric nAChRsubtypes was performed with stably transfected HEK293 cells and [³H]epibatidine (0.5 nM). KD = 0.021 nM (α2β2-nAChR). KD = 0.084 nM (α2β4-nAChR). KD = 0.034 nM (α3β2-nAChR). KD = 0.29 nM (α3β4-nAChR). KD = 0.046 nM (α4β2-nAChR). KD = 0.094 nM (α4β4-nAChR). Xiao, Y., et al. 2004.
c Human 5-HT3 recombinant/HEK293 cells, radiotracer [³H]GR65630 (0.35 nM). KD = 0.5 nM
ntd = not tested.

### α7-nAChR Assays

The α7-nAChR assays for 7a-e, 10, 11, 14, and 15 were performed using a commercial assay consisting of rat cortical membranes (rich in α7-nAChR) in competition against 0.1 nM [¹²⁵I]α-bungarotoxin, an α7-nAChR antagonist with a KD of 0.7 nM. These assays were performed independently in duplicate, each twice (Table 2). Assays for two reference compounds, methyllycaconitine (MLA), a conventional reference α7-nAChR antagonist, and compound 5, Schrimpf, et al., Bioorg. Med. Chem. Lett. (2012), a lead of our series, were also performed (Table 3). The new series of fluoro isomers 7a-d exhibited high binding affinity at α7-nAChRs with Kᵢ values in the range 0.3-2.5 nM, whereas the binding affinity of isomer 7e was lower (Table 2). The Kᵢ values of the fluoro derivatives 7a-d (Table 2) were better than that of the conventional reference α7-nAChR ligand MLA (Table 3). Among all fluoro isomers compound 7a manifested the best α7-nAChR binding affinity that was an order of magnitude better than MLA and at least comparable to the nonfluorinated lead 5 (Tables 2 and 3).

TABLE 3
Inhibition of In Vitro Binding Affinities
(Kᵢ, nM) of Reference Compounds toward α7-nAChR^{a}

| Compound | α7-nAChR |
|---|---|
| MLA | 2.91 ± 0.76 |
| | n = 9 |
| 2 | 20.4 |
| 3 | 38.0 |
| 4 | 3.3 |
| 5 | 0.30, 0.50 |

^{a}The binding assay conditions are the same as those in Table 2.

Within the series 7a-e, two fluoro derivatives 7a and 7c were selected for further evaluation. This selection was based on the high α7-nAChR binding affinity and selectivity of 7a and 7c (see Table 2) and the suitability of these compounds for radiolabeling with [¹⁸F]. The radiolabeling of [¹⁸F]7a and [¹⁸F]7c was anticipated to be accomplished by a direct nucleophilic substitution (SNAr) with [¹⁸F]fluoride via the nitro 10 and 11 or iodo derivatives 14 and 15, respectively. The leaving nitro groups in 10 and 11 or iodo groups in 14 and 15 are activated for SNAr fluorination by the powerful electron-withdrawing SO₂Ar on the ortho and para positions, respectively. Smith and March, Advanced Organic Chemistry (2007); Kubinyi, The Quantitative Analysis of Structure—Activity Relationships. In Burger's Medicinal Chemistry and Drug Discovery (1995); Miller, et al., Angew. Chem., Int. Ed. (2008); Hudlicky and Pavlath, Chemistry of Organic Fluorine Compounds II: A Critical Review (1995).

No example of fluorination of nitro or iododibenzothiophene 5,5-dioxides has been found in the literature, but the structural analogue of 11, 4,4-sulfonylbis(p-nitrobenzene), has been converted to the corresponding fluoro derivative with good yield. Clark and Wails, J. Fluorine Chem. (1995).

The fluoro derivative 7b that also exhibited high α7-nAChR binding affinity was not selected for further studies because the activating SO₂Ar was located on the meta position to the leaving group and direct radiolabeling of [¹⁸F]7b via its nitro or iodo derivative was less likely.

The potential precursors 10, 11, 14, and 15 for ¹⁸F fluorination of [¹⁸F]7a and [¹⁸F]7c were studied in the same α7-nAChR inhibition binding assay. The nitro compounds 10 and 11 exhibited α7-nAChR binding affinities comparable to those of the corresponding fluorides 7a and 7c, whereas the binding affinities of iodo derivatives 14 and 15 were lower. Currently, there is no conventional in vitro competition binding assay for α7-nAChR. Different research groups use different radioligands ([¹²⁵I]α-bungarotoxin, [³H]α-bungarotoxin, [³H]MLA, [¹²⁵I]iodo-MLA, [³H]A-585539, and the like) and different sources of receptor tissue (cell lines, brain, adrenal glands) under different conditions for this assay. Ettrup, et al., J. Nucl. Med. (2011); Deuther-Conrad, et al., Eur. J. Nucl. Med. Mol. Imaging (2011); Xiao, Y., et al., Acta Pharmacol. Sin. (2009); Anderson, et al., J. Pharmacol. Exp. Ther. (2008); Navarro, et al., J. Med. Chem. (2000).

It is not surprising that the difference in the Kᵢ values for the same compound under different assay conditions can exceed an order of magnitude. Anderson, et al., J. Pharmacol. Exp. Ther. (2008); Navarro, et al., J. Med. Chem. (2000). Therefore, a direct comparison of Kᵢ values of the previously published α7-nAChR ligands with compounds of our new series is not practical.

For the purpose of comparison, the Kᵢ values were determined for the three most recently published α7-nAChR PET radioligands [11C]2, Ettrup, et al., J. Nucl. Med. (2011), Deuther-Conrad, et al., Eur. J. Nucl. Med. Mol. Imaging (2011), and [¹⁸F]4, Ravert et al., Nucl. Med. Biol. (2013). See Table 3, under the same assay conditions as those of the presently disclosed series (Table 2). It was noteworthy that the α7-nAChR binding affinities of the best compounds of the presently disclosed series 7a and 7c were substantially better than those of the previous radioligands.

### Heteromeric nAChR Subtypes Assays

The heteromeric nAChR subtypes assays (α2β2-, α2β4-, α3β2-, α3β4-, α4β2-, α4β4-nAChR) were performed in our laboratories using membrane preparations from HEK293 cells expressing the transfected nAChR under test in competition with 0.5 nM [³H]epibatidine to investigate the specificity of the ligand for each receptor (Table 2). The heteromeric nAChR Kᵢ values of the tested compounds 7a, 7c-e, and 15 were substantially greater than the corresponding α7-nAChR Kᵢ values, indicating a high α7-/heteromeric-nAChR subtype selectivity of all studied compounds (Table 2). Thus, the fluoro isomer 7a with the best α7-nAChR binding affinity also manifested an excellent selectivity versus heteromeric nAChR including the main cerebral subtype α4β2-nAChR (Table 2). Interestingly, the α7/α4β2 selectivity of iodo derivative 15 is 10 times lower than the corresponding fluoro derivative 7c.

### 5-HT3 Assay

The in vitro binding affinity of the most promising members of the series, compounds 7a and 7c, at the 5-HT3 receptor was determined commercially using membrane preparations from HEK293 cells expressing transfected human 5-HT3R in competition with 0.35 nM [³H]GR65630, a 5-HT3R antagonist with a KD of 0.5 nM. The assay demonstrated that fluoro compounds 7a and 7c manifest relatively low 5-HT3 binding affinities and they are highly α7-nAChR/5HT3 selective (Table 2).

### Lipophilicity of 7a and 7c

Lipophilicity (log D7.4) is considered an important property of CNS radioligand because it has been linked to the blood-brain barrier permeability and nonspecific binding. Kulak, et al., Eur. J. Neurosci. (2006); Eckelman, et al., J. Nucl. Med. (1979); Tanibuchi, et al., Brain Res. (2010). The lipophilicity values for 7a and 7c (log D7.4=2.0) were calculated with ACD Labs Structure Designer Suite (ACD Labs, Toronto, Canada) and fall within the conventional range for CNS PET radioligands.

### Radiochemistry

The fluoro isomers 7a and 7c that exhibited the highest binding affinity within the series with fluorine-18 have been radiolableled. The radiosyntheses were performed remotely in one step by 1,10-diaza-4,7,13,16,21,24-hexaoxabicyclo[8.8.8]hexacosane (Kryptofix-222) assisted radiofluorination of the respective nitro precursors 10 and 11 (Scheme 7) or iodo precursors 14 and 15 using a radiochemistry synthesis module (Microlab, GE) followed by the semipreparative HPLC separation and formulation of [¹⁸F]7a and [¹⁸F]7c as sterile apyrogenic solutions in 7% ethanolic saline. It is noteworthy that the radiotracer product yields from iodo precursors 14 and 15 were substantially lower than those of the nitro precursors 10 and 11. The conventional Kryptofix-222/potassium carbonate assisted radiofluorination of both iodo derivatives 14 and 15 in DMSO at 130-180° C. produced [¹⁸F]7a and [¹⁸F]7c with radiochemical yields below 0.5%, and this radiosynthesis pathway was not optimized further (not shown). The radiofluorination of nitro derivative 10 or 11 (Scheme 7) in the presence of Kryptofix-222/potassium carbonate at 160° C. produced [¹⁸F]7a or [¹⁸F]7c in a slightly better yield (2-3%). Further optimization of this radiosynthesis suggested that both final products [¹⁸F]7a and [¹⁸F]7c rapidly decomposed in the DMSO reaction solution in the presence of highly basic K₂CO₃, but the radiochemical yield was improved if the less basic potassium oxalate was used. In the presence of potassium oxalate, the final products [¹⁸F]7a and [¹⁸F]7c were prepared under similar reaction conditions with comparable radiochemical yields of 16±6% (n=14) (nondecay-corrected), with specific radioactivities in the range 330-1260 GBq/μmol (9-34 Ci/μmol) and a radiochemical purity greater than 99%. The nitro precursors 10 and 11 that exhibited substantial α7-nAChR binding affinity (Table 2) were fully separated by preparative HPLC and were not detected by analytical HPLC in the final products [¹⁸F]7a and [¹⁸F]7c (data not shown).

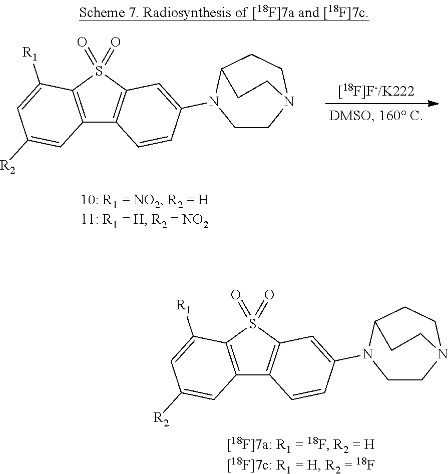

The iodo isomer 10 also has been radiolabeled. Even though it exhibited lowest binding affinity compared to the fluoro isomers 7a and 7b, Iodine-125 has a longer half-life than Fluorine-18, which make the iodine radiolabeled compounds useful for certain scanning procedure that last longer than a few hours. The radiosynthesis was performed in one step by radioidination of the nitro precursor (Scheme 8), followed by the semipreparative HPLC separation and formulation of [¹²⁵I]14 as sterile apyrogenic solution in 7% ethanolic saline.

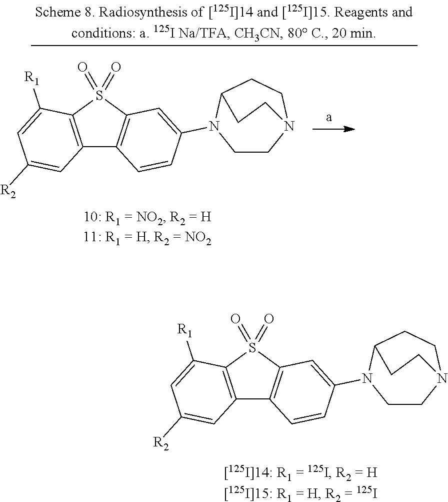

### Typical Procedure for Reduction of Nitro Derivatives to Anilines 12, 13, 19, 26, 27

A mixture of nitro compound (1 mmol), iron powder (4 mmol), ammonium chloride (1.2 mmol) in methanol (6 mL), THF (6 mL), and water (2 mL) was heated to reflux (80° C.) for 3 h. The resulting mixture was diluted with ethanol and concentrated and dried under vacuum. The residue was purified by silica gel column chromatography (CHCl3/i-PrOH/Et3N 10:1:0.1 to 10:30:4) to give the corresponding aniline derivative.

### Typical Procedure for Bromination

N-Bromosuccinimide (NBS) (1 mmol) was added to a solution of the starting 1,4-dibenzothiophene derivative (1 mmol) in concentrated H₂SO₄ (3.6 mL) at room temperature. After 24 h, the solution was carefully poured into ice/water. The solids were filtered and washed with water and methanol. The obtained solids were recrystallized from 95% EtOH to afford the bromo compounds.

### Typical Procedure for Oxidation of 1,4-Dibenzothiophene Derivatives

1,4-Dibenzothiophene derivative (1 mmol) was dissolved in glacial acetic acid (2.8 mL) at room temperature. Aqueous hydrogen peroxide (30%, 1.4 mL) was added in small portions to the stirred solution. The addition of H₂O₂ resulted initially in some precipitation. The mixture was stirred at 60° C. for 24 h, then cooled to room temperature. The solid was filtered off, sequentially washed with 70% aqueous acetic acid, then 30% aqueous acetic acid, then water, and dried to afford the title compound.

### 3-Bromo-6-fluorodibenzo[b,d]thiophene 5,5-Dioxide (6a)

The typical procedure for oxidation of 1,4-dibenzothiophene was followed, starting with 20 (600 mg, 2.13 mmol). The title compound 6a (648 mg, 97%) was obtained as white crystals. 1H NMR (CDCl₃, 400 MHz) δ 7.97 (s, 1H), 7.81 (dd, J=12.0, 1.8 Hz, 1H), 7.68 (d, J=8.0 Hz, 1H), 7.66 (dd, J=8.0, 4.0 Hz, 1H), 7.59 (d, J=8.0 Hz, 1H), 7.24 (t, J=8.0 Hz, 1H).

### 3-Bromo-7-fluorodibenzo[b,d]thiophene 5,5-Dioxide (6b)

A mixture of 26 (620 mg, 2 mmol) and 48% tetrafluoroboric acid (HBF4) (4 mL) was stirred at 0-5° C. for 10 min. A cold solution of sodium nitrite (204 mg in 0.8 mL of water, 3 mmol) was added dropwise with stirring. After the mixture was stirred for 1 h at 0-5° C. the precipitated intermediate diazonium tetrafluoroborate was collected by filtration, washed with cold tetrafluoroboric acid (5%) and water and Et₂O, and dried under vacuum. The diazonium tetrafluoroborate was boiled in xylene (135° C.) for 120 min. The solvent was evaporated under reduced pressure. The residue was extracted with a mixture of chloroform and water. The chloroform layer was separated and concentrated. The residue was chromatographed on silica gel using hexanes-EtOAc (4:1) as eluent to give 6b as a pale yellow solid (330 mg, 53%). 1H NMR (CDCl₃, 400 MHz) δ 7.96 (d, J=2.0 Hz, 1H), 7.81-7.77 (m, 2H), 7.64 (d, J=8.0 Hz, 1H), 7.54 (dd, J=8.0, 4.0 Hz, 1H), 7.40-7.35 (m, 1H). 7-Bromo-2-fluorodibenzo[b,d]thiophene 5,5-Dioxide (6c). A mixture of 27 (310 mg, 1 mmol) and 48% tetrafluoroboric acid (HBF₄) (2 mL) was stirred at 0-5° C. for 10 min. A cold solution of sodium nitrite (102 mg, 1.5 mmol) in 0.4 mL of water was added dropwise with stirring. After the mixture was stirred for 1 h at 0-5° C., the precipitated diazonium tetrafluoroborate was collected by filtration, washed with cold tetrafluoroboric acid (5%) and water and Et₂O, and dried under vacuum. The diazonium tetrafluoroborate was boiled in xylene (135° C.) for 30 min. The solvent was evaporated under reduced pressure. The residue was treated with chloroform and water. The chloroform layer was separated and concentrated. The residue was chromatographed on silica gel using hexanes-EtOAc (4:1) as eluent to give 6c as a pale yellow solid (156 mg, 50%). 1H NMR (DMSO-d₆, 400 MHz) δ 8.38 (d, J=4.0 Hz, 1H), 8.23-8.18 (m, 2H), 8.13-8.07 (m, 2H), 7.54 (t, J=8.0 Hz, 1H).

### 3-Bromo-4-fluorodibenzo[b,d]thiophene 5,5-Dioxide (6d) and 1-Bromo-4-fluorodibenzo[b,d]thiophene 5,5-Dioxide (6e)

The typical procedure for bromination was followed, starting with 29 (905 mg, 3.86 mmol). Separation of the crude reaction product by silica gel chromatography using hexanes/ethyl acetate (5:2) yielded two isomers 6d (285 mg, 0.91 mmol, 23.6%) and 6e (160 mg, 0.51 mmol, 13%). The isomer 6e was in the first chromatography fraction, whereas 6d was in the second fraction. 6d: Rf=0.31 (hexanes/EtOAc 2:1); 1H NMR (CDCl3, 400 MHz) δ 7.86-7.80 (m, 3H), 7.70 (t, J=8.0 Hz, 1H), 7.63 (d, J=8.0 Hz, 1H), 7.49 (d, J=8.0 Hz, 1H). 6e: Rf=0.5 (hexanes/EtOAc 2:1); 1H NMR (CDCl3, 400 MHz) δ 8.94 (d, J=8.0 Hz, 1H), 7.90 (d, J=8.0 Hz, 1H), 7.83-7.79 (m, 1H), 7.75 (dd, J=8.0, 4.0 Hz, 1H), 7.67 (d, J=8.0 Hz, 1H), 7.11 (t, J=8.0 Hz, 1H).

### Typical Procedure for Buchwald-Hartwig Cross-Coupling Reaction

### 3-(1,4-Diazabicyclo[3.2.2]nonan-4-yl)-6-fluorodibenzo[b,d]thiophene 5,5-Dioxide (7a)

A catalyst solution was prepared by mixing tris(dibenzylideneacetone)dipalladium (Pd₂(dba)₃, 58 mg, 0.063 mmol; Aldrich) and racemic BINAP (39 mg, 0.125 mmol; Strem) in toluene (4 mL) and heating the mixture to 90° C. for 15 min. The solution was cooled and then added to a mixture of 1,4-diazabicyclo[3.2.2]nonane (200 mg, 1.58 mmol) and 6a (0.492 g, 1.58 mmol), in toluene (12 mL). Cs₂CO₃ (766 mg, 2.4 mmol; Aldrich) was then added, and the reaction mixture was flushed with nitrogen and heated overnight at 80-85° C. After cooling to room temperature, the mixture was concentrated and purified by silica gel flash chromatography (CHCl₃/i-PrOH/Et₃N 10:1:0.2). The title compound 7a (227 mg, 40% yield) was obtained as a yellow solid. 1H NMR (DMSO-d₆, 400 MHz) δ 7.89 (d, J=8.0 Hz, 1H), 7.77 (d, J=8.0 Hz, 1H), 7.73 (t, J=8.0 Hz, 1H), 7.29-7.24 (m, 2H), 7.12 (d, J=8.0 Hz, 1H), 4.19 (s, 1H), 3.70-3.67 (m, 2H), 2.98-2.91 (m, 4H), 2.88-2.82 (m, 2H), 1.99 (m, 2H), 1.72-1.66 (m, 2H); HRMS calculated for C19H2OFN2O2S ([M+H]) 359.1224. found, 359.1240.

### Preparation of 7a·p-TSA Salt

A mixture of 7a (30 mg, 0.084 mmol) and p-toluenesulfonic acid monohydrate (19 mg, 0.099 mmol) was stirred in EtOAc-EtOH (2 mL, 10:1) at room temperature for 2 h. The resulting solid was collected, washed with EtOAc-EtOH (2 mL, 10:1) and EtOAc (3 mL), and dried under vacuum to afford the title compound as a yellow solid (32 mg, 72% yield). 1H NMR (DMSO-d6, 400 MHz) δ 10.10 (s, 1H), 7.99 (d, J=8.0 Hz, 1H), 7.85 (d, J=8.0 Hz, 1H), 7.79-7.73 (m, 1H), 7.49-7.45 (m, 3H), 7.32 (t, J=8.0 Hz, 1H), 7.25 (dd, J=8.0, 4.0 Hz, 1H), 7.12 (br s, 1H), 7.10 (br s, 1H), 4.47 (s, 1H), 3.95-3.93 (m, 2H), 3.49-3.39 (m, 6H), 2.29 (s, 3H), 2.19 (m, 2H), 2.05 (m, 2H). Elemental analysis for C26H27FN2O5S2, calcd: C, 58.85; H, 5.13; N, 5.28. Found: C, 58.57; H, 5.04; N, 5.18.

### 4-(7-Fluorodibenzo[b,d]thiophen-3-yl)-1,4-diazabicyclo-[3.2.2]nonane 5,5-Dioxide (7b)

The typical procedure for Buchwald-Hartwig cross-coupling reaction was followed, starting with 6b (0.2 g, 0.64 mmol). The title compound 7b was obtained as a yellow solid (104 mg, 0.29 mmol, 45% yield). 1H NMR (DMSO-d6, 400 MHz) δ 7.99 (dd, J=8.0, 4.0 Hz, 1H), 7.89 (dd, J=8.0, 3.0 Hz, 1H), 7.86 (d, J=8.0 Hz, 1H), 7.55 (m, 1H), 7.25 (d, J=4.0 Hz, 1H), 7.11 (dd, J=8.0, 4.0 Hz, 1H), 4.17 (s, 1H), 3.66 (m, 2H), 2.99-2.91 (m, 3H), 2.87-2.82 (m, 3H), 2.00-1.97 (m, 2H), 1.71-1.65 (m, 2H). Elemental analysis for C19H19FN2O2S.0.1H2O, calcd: C, 62.37; H, 5.17; N, 7.58. Found: C, 62.25; H, 5.44; N, 7.19.

### 7-(1,4-Diazabicyclo[3.2.2]nonan-4-yl)-2-fluorodibenzo[b,d]-thiophene 5,5-Dioxide (7c)

The typical procedure for Buchwald-Hartwig cross-coupling reaction was followed, starting with 6c (0.226 g, 0.72 mmol), and the title compound 7c (140 mg, 54% yield) was obtained as a yellow solid. 1H NMR (DMSO-d6, 400 MHz) δ 7.93-7.87 (m, 3H), 7.26-7.21 (m, 2H), 7.12 (d, J=8.0 Hz, 1H), 4.20 (s, 1H), 3.71-3.68 (m, 2H), 3.01-2.83 (m, 6H), 2.00 (br s, 2H), 1.73-1.67 (m, 2H); HRMS calculated for C19H2OFN2O2S ([M+H]) 359.1224. found, 359.1241. TSA salt: 1H NMR (DMSO-d6, 400 MHz) δ 10.08 (s, 1H), 8.00-7.94 (m, 3H), 7.48 (d, J=8.0 Hz, 1H), 7.43 (m, 2H), 7.32-7.25 (m, 2H), 7.12 (br, 1H), 7.10 (br, 1H), 4.48 (s, 1H), 3.94 (m, 2H), 3.47-3.38 (m, 6H), 2.29 (s, 3H), 2.19 (m, 2H), 2.06-1.99 (m, 2H). Elemental analysis for C26H27FN2O5S2.0.75H2O, calcd: C, 57.39; H, 5.28; N, 5.15. Found: C, 57.22; H, 5.11; N, 5.12.

### 3-(1,4-Diazabicyclo[3.2.2]nonan-4-yl)-4-fluorodibenzo[b,d]-thiophene 5,5-Dioxide (7d)

The typical procedure for Buchwald-Hartwig cross-coupling reaction was followed, starting with 6d (0.246 g, 0.78 mmol), and the title compound 7d was obtained as a yellow solid (170 mg, 0.47 mmol, 60% yield). Free base: 1H NMR (DMSOd6, 400 MHz) δ 8.06 (d, J=8.0 Hz, 1H), 7.91 (d, J=8.0 Hz, 1H), 7.81 (d, J=8.0 Hz, 1H), 7.75 (t, J=8.0 Hz, 1H), 7.54 (t, J=8.0 Hz, 1H), 7.36 (t, J=8.0 Hz, 1H), 3.82 (s, 1H), 3.43-3.40 (m, 2H), 3.03-3.00 (m, 2H), 2.93-2.89 (m, 4H), 2.00-1.97 (m, 2H), 1.74-1.66 (m, 2H); HRMS calculated for C19H2OFN2O2S ([M+H]) 359.1224. found, 359.1246. TSA salt: 1H NMR (DMSO-d6, 400 MHz) δ 10.16 (s, 1H), 8.12 (br s, 1H), 7.94-7.90 (m, 2H), 7.78 (d, J=8.0 Hz, 1H), 7.59 (d, J=8.0 Hz, 1H), 7.50-7.40 (m, 3H), 7.12 (m, 2H), 4.01 (s, 1H), 3.54-3.38 (m, 6H), 2.30 (s, 3H), 2.19 (s, 2H), 2.07 (s, 2H), 1.09-1.03 (m, 2H). Elemental analysis for C26H27FN2O5S2.0.5H2O, calcd: C, 57.87; H, 5.23; N, 5.19. Found: C, 58.21; H, 5.56; N, 4.88.

### 1-(1,4-Diazabicyclo[3.2.2]nonan-4-yl)-4-fluorodibenzo[b,d]-thiophene 5,5-Dioxide (7e)

The typical procedure for Buchwald-Hartwig cross-coupling reaction was followed, starting with 6e (0.112 g, 0.36 mmol). The title compound 7e was obtained as a yellow solid (52 mg, 0.15 mmol, 40% yield). 1H NMR (CDCl3, 400 MHz) δ 8.50 (d, J=8.0 Hz, 1H), 7.84 (d, J=8.0 Hz, 1H), 7.68 (t, J=8.0 Hz, 1H), 7.55 (t, J=8.0 Hz, 1H), 7.43 (dd, J=8.0, 4.0 Hz, 1H), 7.14 (t, J=8.0 Hz, 1H), 3.66-3.63 (m, 1H), 3.29-3.21 (m, 5H), 3.14-3.09 (m, 2H), 2.16-2.10 (m, 2H), 1.87-1.71 (m, 3H); HRMS calculated for C19H2OFN2O2S ([M+H]) 359.1224. found, 359.1215. Elemental analysis for C19H19FN2O2S.1.5H2O, calcd: C, 59.20; H, 5.75; N, 7.27. Found: C, 58.90; H, 5.76; N, 7.10.

### 3-Bromo-6-nitrodibenzo[b,d]thiophene 5,5-Dioxide (8)

The typical procedure for bromination was followed, starting with 31 (1.96 g, 7.5 mmol), and compound 8 was obtained as a pale brown solid (1.73 g, 77%). 1H NMR (DMSO-d6, 400 MHz) δ 8.70 (d, J=8.0 Hz, 1H), 8.45-8.43 (m, 2H), 8.28 (d, J=8.0 Hz, 1H), 8.14-8.09 (m, 2H). HRMS calculated for C12H6BrNNaO4S ([M+Na]+) 361.9093. found, 361.9080.

### 7-Bromo-2-nitrodibenzo[b,d]thiophene 5,5-Dioxide (9)

The typical procedure for bromination was followed, starting with 24 (1.82 g, 6.95 mmol), and compound 9 (2.1 g, 89%) was obtained as a pale yellow solid. 1H NMR (DMSO-d6, 400 MHz) δ 9.10 (s, 1H), 8.44-8.47 (m, 3H), 8.33 (d, J=8.0 Hz, 1H), 8.11 (dd, J=8.0, 4.0 Hz, 1H).

### 3-(1,4-Diazabicyclo[3.2.2]nonan-4-yl)-6-nitrodibenzo[b,d]-thiophene 5,5-Dioxide (10)

The typical procedure for Buchwald-Hartwig cross-coupling reaction was followed starting with 8 (0.129 g, 0.38 mmol). Note that the reaction mixture was heated at 105° C. for 48 h. The title compound 10 was obtained as a reddish solid (80 mg, 55% yield). 1H NMR (DMSO-d6, 400 MHz) δ 8.40 (d, J=4.0 Hz, 1H), 8.15 (d, J=8.0 Hz, 1H), 7.97 (d, J=8.0 Hz, 1H), 7.94 (d, J=8.0 Hz, 1H), 7.26 (d, J=4.0 Hz, 1H), 7.15 (d, J=4.0 Hz, 1H), 4.21 (s, 1H), 3.71 (m, 2H), 3.00-2.85 (m, 6H), 2.00 (s, 2H), 1.71 (m, 2H); HRMS calculated for C19H2ON3O4S ([M+H]) 386.1169. found, 386.1150. Elemental analysis for C₁₉H₁₉N₃O4S.H2O, calcd: C, 56.56; H, 5.25; N, 10.42. Found: C, 56.65; H, 4.99; N, 10.50.

### 7-(1, 4-Diazabicyclo[3.2.2]nonan-4-yl)-2-nitrodibenzo[b,d]-thiophene 5,5-Dioxide (11)

The typical procedure for Buchwald-Hartwig cross-coupling reaction was followed, starting with 9 (1.83 g, 5.38 mmol). The title compound 11 was obtained as a reddish solid (0.836 g, 61% yield). 1H NMR (DMSO-d6, 400 MHz) δ 8.77 (s, 1H), 8.20-8.12 (m, 3H), 7.32 (d, J=4.0 Hz, 1H), 7.16 (dd, J=8.0, 4.0 Hz, 1H), 4.23 (s, 1H), 3.72 (m, 2H), 3.00-2.88 (m, 6H), 2.00 (br s, 2H), 1.74-1.69 (m, 2H); HRMS calculated for C19H2ON3O4S ([M+H]) 386.1169. found, 386.1152. Elemental analysis for C₁₉H₁₉N₃O4S.1.25H2O, calcd: C, 55.94; H, 5.31; N, 10.30. Found: C, 55.98; H, 5.17; N, 10.15.

### 6-Amino-3-(1,4-diazabicyclo[3.2.2]nonan-4-yl)-dibenzo[b,d]-thiophene 5,5-Dioxide (12)

The typical procedure for reduction of nitro derivatives was followed starting with 10 (0.34 g, 0.88 mmol), and compound 12 (146 mg, 46%) was obtained as a yellow solid. 1H NMR (DMSO-d6, 400 MHz) δ 7.73 (d, J=12.0 Hz, 1H), 7.25 (t, J=8.0 Hz, 1H), 7.11 (br s, 1H), 7.05 (d, J=8.0 Hz, 1H), 6.97 (d, J=4.0 Hz, 1H), 6.62 (d, J=8.0 Hz, 1H), 5.87 (br s, 2H), 4.17 (s, 1H), 3.66 (m, 2H), 2.98-2.91 (m, 6H), 2.01 (s, 2H), 1.72 (m, 2H).

### 2-Amino-7-(1,4-diazabicyclo[3.2.2]nonan-4-yl)dibenzo[b,d]-thiophene 5,5-Dioxide (13)

The typical procedure for reduction of nitro derivatives was followed starting with 11 (0.68 g, 1.76 mmol), and compound 13 was obtained as a yellow solid (585 mg, 93%). 1H NMR (DMSO-d6, 400 MHz) δ 7.67 (d, J=12 Hz, 1H), 7.43 (d, J=12 Hz, 1H), 7.27 (s, 1H), 7.14 (d, J=8.0, 4.0 Hz, 1H), 6.91 (s, 1H), 6.53 (d, J=8.0, 4.0 Hz, 1H), 6.17 (s, 2H), 4.40 (s, 1H), 3.87 (br s, 3H), 3.07 (m, 1H), 2.15 (br s, 3H), 2.02 (br s, 3H), 1.20 (m, 2H).

### 3-(1,4-Diazabicyclo[3.2.2]nonan-4-yl)-6-iododibenzo[b,d]-thiophene 5,5-Dioxide (14)

Compound 12 (143 mg, 0.4 mmol) was dissolved in a mixture of 4 N H₂SO₄ (0.8 mL) and CH₃CN (1 mL), and the solution was cooled to −5° C. Sodium nitrite (55 mg, 0.8 mmol) dissolved in H₂O (0.5 mL) was added dropwise at the same temperature. After the mixture was stirred for 60 min a solution of diazonium salt was formed. To a mixture of CuI (268 mg, 1.4 mmol) and saturated KI solution (2.5 mL) at 70° C. was added the above prepared solution of diazonium salt dropwise over 10 min, and the mixture was further stirred at 70° C. for 30 min. The reaction mixture was cooled, and 28% ammonia solution was added (2 mL). The aqueous suspension was repeatedly extracted with CHCl₃ and the combined organic layers were washed with brine (10 mL), dried (Na₂SO₄), and concentrated in vacuo. The crude product was purified by flash chromatography on silica gel (CHCl3/i-PrOH/Et3N 10:1:0.1 to 3:1:0.2) to give 14 (28 mg, 15%). 1H NMR (DMSO-d6, 400 MHz) δ 7.96 (d, J=8.0 Hz, 1H), 7.92 (d, J=4.0 Hz, 1H), 7.81 (d, J=8.0 Hz, 1H), 7.38 (t, J=8.0 Hz, 1H), 7.32 (d, J=4.0 Hz, 1H), 7.18 (d, J=8.0 Hz, 1H), 4.34 (s, 1H), 3.82 (m, 2H), 3.22-3.18 (m, 6H), 2.09 (m, 2H), 1.87 (m, 2H). Elemental analysis for C19H19IN2O2S.2.5H2O, calcd: C, 44.63; H, 4.73; N, 5.48. Found: C, 44.88; H, 4.41; N, 5.48.

### 7-(1,4-Diazabicyclo[3.2.2]nonan-4-yl)-2-iododibenzo[b,d]-thiophene 5,5-Dioxide (15)

Compound 13 (285 mg, 0.8 mmol) was dissolved in a mixture of 4 N H₂SO₄ (1.5 mL) and CH₃CN (2 mL), and the solution was cooled to −5° C. NaNO₂ (110 mg, 1.6 mmol, 2 equiv) dissolved in H₂O (1 mL) was added dropwise at the same temperature. After the mixture was stirred for 60 min a solution of diazonium salt was formed. To a mixture of CuI (536 mg, 2.8 mmol, 3.5 equiv) and saturated KI solution (2.5 mL) at 70° C. was added above-prepared solution of diazonium salt dropwise over 10 min and further stirred at 70° C. for 30 min. The reaction mixture was cooled, and saturated NH₄OH was added (4 mL). The aqueous suspension was repeatedly extracted with CHCl₃, and the combined organic layers were washed with brine (10 mL), dried (Na₂SO₄), and concentrated in vacuo. The crude product was purified by flash chromatography on silica gel (CHCl3/i-PrOH/Et3N 10:1:0.1 to 3:1:0.2) to give 15 (75 mg, 20%). 1H NMR (DMSO-d6, 400 MHz) δ 8.40 (d, J=4.0 Hz, 1H), 7.93 (d, J=8.0 Hz, 1H), 7.78 (dd, J=8.0, 1.8 Hz, 1H), 7.60 (d, J=12.0 Hz, 1H), 7.25 (d, J=1.8 Hz, 1H), 7.11 (dd, J=8.0, 4.0 Hz, 1H), 4.20 (s, 1H), 3.70 (m, 2H), 2.98-2.88 (m, 6H), 2.00 (s, 2H), 1.72 (m, 2H); HRMS calculated for C19H2OIN2O2S ([M+H]) 467.0285. found, 467.0306. Elemental analysis for C19H21IN2O3S, calcd: C, 47.12; H, 4.37; N, 5.78. Found: C, 47.24; H, 4.53; N, 5.87.

### (5-Bromo-2-nitrophenyl)(2-fluorophenyl)sulfane (18)

Cesium carbonate (4.3 g, 13.2 mmol) was added to a solution of 4-bromo-2-fluoronitrobenzene 16 (2.42, 11 mmol, Aldrich) and 2-fluorobenzene thiol 17 (1.4 g, 11 mmol, Aldrich) in DMF (60 mL), and the mixture was stirred for 5 h at room temperature. Water (200 mL) and ethyl acetate (100 mL) were added. The organic layer was separated and washed sequentially with water (100 mL) and then brine (100 mL). The organic phase was separated, dried, and concentrated to yield a yellow solid that was purified by silica gel chromatography (hexanes/EtOAc 8:1 to 3:1) to give 18 (2.88 g, 80%). 1H NMR (CDCl3, 400 MHz) δ 8.17 (d, J=8.0 Hz, 1H), 7.68-7.60 (m, 2H), 7.41-7.29 (m, 3H), 6.95 (s, 1H).

### 4-Bromo-2-((2-fluorophenyl)thio)aniline (19)

The typical procedure for reduction of nitro derivatives was followed, starting with 18 (3.2 g, 9.75 mmol), and the title compound 19 was obtained as a brown solid (2.46 g, 85%). 1H NMR (CDCl3, 400 MHz) δ 7.59 (d, J=4.0 Hz, 1H), 7.33 (dd, J=8.0, 4.0 Hz, 1H), 7.21-7.15 (m, 1H), 7.10-7.00 (m, 2H), 6.92-6.87 (m, 1H), 6.69 (d, J=8.0 Hz, 1H), 4.37 (br s, 2H).

### 3-Bromo-6-fluorodibenzo[b,d]thiophene (20)

Compound 19 (1.18 g, 3.96 mmol) was dissolved in 37% HCl (11 mL), and the solution was cooled below 5° C. To this reaction mixture, sodium nitrite (408 mg, 5.93 mmol) was added slowly at a temperature below 5° C. After addition, the mixture was stirred for 30 min below 5° C. Then sodium tetrafluoroborate (865 mg, 7.92 mmol) was added, and the reaction mixture was stirred for another 30 min at a temperature below 5° C. This reaction solution was then added to the stirred solution of copper(I) oxide (1.14 mg, 7.92 mmol) in 0.1 N sulfuric acid (390 mL) at 35-40° C. The reaction mixture was stirred for 15-30 min. Ethyl acetate was added to the reaction mixture, and the mixture was filtered to remove inorganic compound. The filtrate was then extracted with ethyl acetate (3×120 mL). The organic extract was washed with water followed by brine and then concentrated under vacuum. The residue was purified by silica gel chromatography (hexanes) to give 20 (600 mg, 54%). 1H NMR (CDCl3, 400 MHz) δ 8.04 (d, J=4.0 Hz, 1H), 8.02 (d, J=8.0 Hz, 1H), 7.93 (d, J=8.0 Hz, 1H), 7.62 (dd, J=8.0, 4.0 Hz, 1H), 7.47 (ddd, J=12.0, 8.0, 4.0 Hz, 1H), 7.22 (t, J=8.0 Hz, 1H).

### 3-Nitrodibenzo[b,d]thiophene 5,5-Dioxide (23)

Dibenzo-[b,d]thiophene 5,5-dioxide 21 (10 g, 46 mmol, Aldrich) was slowly added to a stirred mixture of glacial acetic acid (34 mL) and sulfuric acid (96%, 34 mL). The slurry was stirred, and red fuming nitric acid (36 mL) was added dropwise over a period of 90 min at temperature −5° C. to 5° C. The slurry was stirred for another 30 min and poured over ice. The precipitate was filtered, rinsed with water, and dried at room temperature. The crude product was recrystallized with acetonitrile to give 23 as yellow crystals (8.7 g, 72%). 1H NMR (DMSO-d6, 400 MHz) δ 8.84 (d, J=8.0 Hz, 1H), 8.65 (dd, J=8.0, 2.0 Hz, 1H), 8.50 (d, J=8.0 Hz, 1H), 8.39 (d, J=8.0 Hz, 1H), 8.13 (d, J=8.0 Hz, 1H), 7.93 (t, J=8.0 Hz, 1H), 7.81 (t, J=8.0 Hz, 1H).

### 2-Nitrodibenzo[b,d]thiophene 5,5-Dioxide (24)

The typical procedure for oxidation of 1,4-dibenzothiophene was followed starting with 22 (489 mg, 2.13 mmol, Oakwood Chemical), and the title compound 24 (510 mg, 90%) was obtained as white crystals. 1H NMR (CDCl3, 400 MHz) δ 8.66 (d, J=4 Hz, 1H), 8.43 (dd, J=8, 4 Hz, 1H), 8.04 (d, J=8 Hz, 1H), 7.96 (d, J=8 Hz, 1H), 7.92 (d, J=8 Hz, 1H), 7.79 (t, J=8 Hz, 1H), 7.69 (t, J=8.0 Hz, 1H).

### 3-Bromo-7-nitrodibenzo[b,d]thiophene 5,5-Dioxide (25)

The typical procedure for bromination was followed starting with 23 (2.59 g, 9.9 mmol), and brown solid was obtained and recrystallized with benzene to yield 25 as a yellow solid (1.73 g, 51%). 1H NMR (DMSO-d6, 400 MHz) δ 8.89 (d, J=4.0 Hz, 1H), 8.67 (dd, J=8.0, 3.0 Hz, 1H), 8.52-8.49 (m, 2H), 8.35 (d, J=8.0 Hz, 1H), 8.15 (dd, J=8.0, 2.0 Hz, 1H).

### 7-Bromodibenzo[b,d]thiophen-3-amine 5,5-Dioxide (26)

A solution of stannous chloride dihydrate (12.4 g, 56 mmol) in 37% hydrochloric acid (21 mL) was added to a mixture of 25 (1.7 g, 5 mmol) in glacial acetic acid (50 mL). The reaction mixture was stirred at 100° C. for 60 min and cooled to 5° C. The precipitate was filtered off, rinsed with water on the filter, and dispersed in water. The dispersion was made basic (pH 10) by addition of an excess of 1 M sodium hydroxide and stirred for 3 h. The precipitate was filtered off, rinsed with water, and dried overnight on the filter to yield 26 (0.7 g, 45%) as a pale white solid. 1H NMR (DMSO-d6, 400 MHz) δ 8.12 (s, 1H), 7.87-7.77 (m, 3H), 6.95 (s, 1H), 6.87 (dd, J=8, 4 Hz, 1H), 6.20 (br s, 2H).

### 2-Amino-7-bromodibenzo[b,d]thiophene 5,5-Dioxide (27)

The typical procedure for reduction of nitro derivatives was followed starting with 9 (0.60 g, 1.76 mmol). The title compound 27 (496 mg, 91%) was obtained as a white solid. 1H NMR (DMSO-d6, 400 MHz) δ 8.16 (d, J=4 Hz, 1H), 7.93 (d, J=8.0 Hz, 1H), 7.87 (d, J=12 Hz, 1H), 7.56 (d, J=8 Hz, 1H), 7.08 (s, 1H), 6.71 (d, J=8 Hz, 1H), 6.36 (br s, 2H).

### 4-Fluorodibenzo[b,d]thiophene 5,5-Dioxide (29)

The typical procedure for oxidation of 1,4-dibenzothiophene was followed starting with 4-fluorodibenzo[b,d]thiophene 28, Nag and Jenks, J. Org. Chem. (2005), (1.62 g, 8 mmol). The title compound 29 (1.8 g, 96%) was obtained as white crystals. 1H NMR (CDCl3, 400 MHz) δ 7.85 (d, J=8.0 Hz, 1H), 7.82 (d, J=8.0 Hz, 1H), 7.71-7.57 (m, 4H), 7.20 (t, J=8.0 Hz, 1H).

### 4-Nitrodibenzo[b,d]thiophene 5,5-Dioxide (31)

The typical procedure for oxidation of 1,4-dibenzothiophene was followed starting with 30, Manna, et al., Org. Lett. (2012), (1.08 g, 4.71 mmol). The final compound 31 (1.1 g, 90%) was obtained as pale yellow crystals. 1H NMR (DMSO-d6, 400 MHz) δ 8.69 (d, J=8.0 Hz, 1H), 8.42 (d, J=8.0 Hz, 1H), 8.33 (d, J=8.0 Hz, 1H), 8.11 (t, J=8.0 Hz, 1H), 8.07 (d, J=8.0 Hz, 1H), 7.88 (t, J=8.0 Hz, 1H), 7.77 (t, J=8.0 Hz, 1H). HRMS calculated for C12H7NNaO4S ([M+Na]) 283.9988. found, 283.9994.

### Radiosynthesis of [¹⁸F]7a and [¹⁸F]7e

The same radiolabeling method was used for both radioligands [¹⁸F]7a and [¹⁸F]7c. A solution of the [¹⁸F]fluoride, 15-20 mg of Kryptofix 222, and 1-2 mg of K₂C₂O₄ in 1 mL of 50% aqueous acetonitrile was added to a reaction vessel of a GE MicroLab box. The mixture was heated at 120-135° C. under a stream of argon, while water was evaporated azeotropically after the addition of 2 mL of CH₃CN. A solution of the corresponding nitro precursor (10 or 11) (2 mg) in anhydrous DMSO (0.8 mL) was added to the reaction vessel and heated at 160° C. for 12 min. The reaction mixture was cooled, diluted with 0.7 mL of water, and injected onto the reverse-phase semipreparative HPLC column (Table 6). The radioactive peak was collected in 50 mL of HPLC water. The water solution was transferred through an activated Waters C-18 Oasis HLB light solid-phase extraction (SPE) cartridge. After the SPE was washed with 10 mL of saline, the product was eluted with a mixture of 1 mL of ethanol and 0.04 mL of 1 N HCl through a 0.2 μm sterile filter into a sterile, pyrogen-free multidose vial and 10 mL of 0.9% saline and 0.05 mL of sterile 8.4% solution sodium bicarbonate were added through the same filter. The final products [¹⁸F]7a and [¹⁸F]7c were then analyzed by analytical HPLC (Table 6) using a UV detector at 340 nm to determine the radiochemical purity and specific radioactivity at the time synthesis ended. The total synthesis time including QC was 70-80 min.

TABLE 6
HPLC Conditions for [¹⁸F]7a and [¹⁸F]7c

| | | | flow rate, | product retention | nitro precursor |
|---|---|---|---|---|---|
| | column | mobile phase | mL/min | time, min | retention time, min |
| [¹⁸F]7a, preparative | XBridge C18 column, | CH₃OH/CH₃CN/H₂O/Me₃N | 12 | 32 | 21 |
| | 10 μm (250 mm × 10 mm) | 260:120:620:2 | | | |
| [¹⁸F]7a, analytical | XBridge C18 column, | CH₃CN/ H₂O/Et₃N | 2 | 7.4 | 5.5 |
| | 5 μm (250 mm × 4.6 mm) | 390:610:1 | | | |
| [¹⁸F]7c, preparative | XBridge C18 column, | CH₃CN/H₂O/NH₃ | 10 | 20 | 27 |
| | 10 μm (150 mm × 10 mm) | 280:720:1 | | | |
| [¹⁸F]7c, analytical | XBridge C18 column, | CH₃CN/H₂O/NH₃ | 2 | 3.4 | 5.2 |
| | 3.5 μm (100 mm × 4.6 mm) | 380:620:1 | | | |

### Radiosynthesis of [^{125I]}3-(1 A-Diazabicyclo[3.2.2]nonan-4-yl)-6-iododibenzo[b,d]thiophene 5,5-Dioxide ([¹²⁵I]14)

To a solution of A-55 (1 mg, 0.002 mmol) in CH3CN (0.1 mL) was added 7 mCi of Na ¹²⁵I in 0.1 N NaOH at room temperature, followed by TFA (10-μL, 67.5 equiv.). The mixture was heated at 80″ C in a sand bath for 20 min. The reaction mixture was cooled and was diluted with 50% CH3CN (50-μL) and applied to reverse phase semipreparative HPLC column. The radioactive peak was collected and was transferred through an activated Waters C-18 Oasis HLB light solid-phase extraction (SPE) cartridge. After the SPE was washed with 10 mL of saline, the product was eluted with a mixture of 1 mL of ethanol and 0.04 mL of 1 N HCl through a 0.2 11 m sterile filter into a sterile, pyrogen-free multidose vial and 10 mL of 0.9% saline and 0.05 mL of sterile 8.4% solution sodium bicarbonate were added through the same filter. The final product [¹²⁵I]14 was then analyzed by analytical HPLC using a UV detector at 340 nm to determine the radiochemical purity and specific radioactivity at the time synthesis ended. The total synthesis time including QC was 70-80 min.

Preparative HPLC condition: Luna prep column, 250×10 mm, 10 micron, 280/720/1 CH3CN/H20ITFA, 6 mL/min, iodide product [125I]14 T:=28 min, precursor can not be washed out. Iodo product standard: Luna analytical 250×4.6 mm, 10 micron (Gao), 50/50/0.1 CH3CN/H20ITFA, 2 mL/min, T:=8.44 min.

### In Vitro Binding Assay. α7-nAChR Assay with Rat Brain Membranes

The assay was done commercially by Caliper PerkinElmer (Hanover, Md.). In brief, rat cortical membranes were incubated with [¹²⁵I]α-bungarotoxin (KD=0.7 nM) at 0.1 nM in a buffer consisting of 50 mM Tris, 120 mM NaCl, 5 mM KCl, 2 mM CaCl₂, 1 mM MgCl₂, 0.003 mM atropine sulfate at pH 7.4 for 150 min at 0° C.63 The binding was terminated by rapid vacuum filtration of the assay contents onto GF/C filters presoaked in PEI. Radioactivity trapped onto the filters was assessed using a γ-counter. Nonspecific binding was defined as that remaining in the presence of 1 nM α-bungarotoxin. The assays were done two times independently, each in duplicate, at multiple concentrations of the test compounds. Binding assay results were analyzed using a one-site competition model, and IC₅₀ curves were generated based on a sigmoidal dose response with variable slope. The Kᵢ values were calculated using the Cheng-Prusoff equation. Methyllycaconitine (MLA) was used as a reference compound in all assays.

### HEK 293 Cell Culture and Stable Transfections (Heteromeric nAChR)

HEK 293 cells (ATCC CRL 1573) were maintained at 37° C. with 5% CO₂ in a humidified incubator. Growth medium for the HEK 293 cells was the minimum essential medium supplemented with 10% fetal bovine serum, 100 units/mL penicillin G, and 100 ng/mL streptomycin. Transfections of these cells and selection and establishment of stable cell lines were carried out as described previously. Xiao, et al., Acta Pharmacol. Sin. (2009); Xiao and Kellar, J. Pharmacol. Exp. Ther. (2004).

### Membrane Homogenate Preparation (Heteromeric nAChR)

Membrane homogenates for ligand binding assays were made as described previously. Xiao, et al., Acta Pharmacol. Sin. (2009); Xiao and Kellar, J. Pharmacol. Exp. Ther. (2004). Briefly, cultured cells at >90% confluency were removed from the culture flask (80 cm²) with a disposable cell scraper and placed in 10 mL of 50 mM Tris-HCl buffer (pH 7.4, 4° C.). The cell suspension was centrifuged at 1000 g for 5 min, and the pellet was collected. The cell pellet was then homogenized in 10 mL of buffer with a Polytron homogenizer for 20 s and centrifuged at 35000 g for 10 min at 4° C. Membrane pellets were resuspended in fresh buffer.

### Binding to Heteromeric nAChR

Binding to heteromeric nAChR subunit combinations, which represent possible heteromeric nAChRs, was measured with 0.5 nM [3H]epibatidine in HEK cells expressing these subunits (KD=0.021 nM (α2β2-nAChR), KD=0.084 nM (α2β4-nAChR), KD=0.034 nM (α3β2-nAChR), KD=0.29 nM (α3β4-nAChR), KD=0.046 nM (α4β2-nAChR), KD=0.094 nM (α4β4-nAChR)).55 Aliquots of the membrane homogenates containing 30-200 μg of protein were used for the binding assays, which were carried out in a final volume of 100 μL in borosilicate glass tubes. After incubation at 24° C. for 2 h, the samples were collected with a cell harvester (Brandel M-48) onto Whatman GF/C filters prewet with 0.5% polyethylenimine. After the samples were harvested, the filters were washed three times with 5 mL of 50 mM Tris-HCl buffer and then counted in a liquid scintillation counter. Nonspecific binding was measured in samples incubated in parallel containing 300 μM nicotine for [³H]epibatidine binding. Specific binding was defined as the difference between total binding and nonspecific binding. Data from these competition binding assays were analyzed using Prism 5 (GraphPad Software, San Diego, Calif.).

### 5-HT3(h) Binding Assay

The assay was done commercially by Caliper PerkinElmer (Hanover, Md.) using recombinant HEK293 cells and 0.35 nM [³H]GR65630 (KD=0.5 nM).

### Biodistribution Studies in CD-1 Mice. Baseline Study

Male CD-1 mice weighing 25-30 g from Charles River Laboratories (Wilmington, Mass.) were used for biodistribution studies. The animals were sacrificed by cervical dislocation at various times following injection of [¹⁸F]7a or [¹⁸F]7c (70 μCi, specific radioactivity 8000-12000 mCi/μmol, in 0.2 mL of saline) into a lateral tail vein, three animals per time point. The brains were rapidly removed and dissected on ice. The brain regions of interest were weighed, and their radioactivity content was determined in an automated 7-counter with a counting error below 3%. Aliquots of the injectate were prepared as standards, and their radioactivity content was counted along with the tissue samples. The percent of injected dose per gram of tissue (% ID/g tissue) was calculated. All experimental protocols were approved by the Animal Care and Use Committee of the Johns Hopkins Medical Institutions.

### Self-Blockade of [¹⁸F]7a Binding with 7a

In vivo saturation blockade studies were done by iv coadministration of the radiotracer [¹⁸F]7a (70 μCi, SA=9200 mCi/μmol, 0.2 mL) with various doses of “cold” 7a per animal (0 μg (vehicle), 0.0048 μg, 7.2 μg). Compound 7a was dissolved in saline at pH 5.5. At 90 min after administration of the tracer and blocker, brain tissues were harvested, and their regional radioactivity content was determined. The self-blockade of [¹⁸F]7c with 7c was done similarly.

### Blockade of [¹⁸F]7a Binding with 1

In vivo α7-nAChR receptor blocking studies were done by intravenous coadministration of the radiotracer [¹⁸F]7a (70 μCi, SA=7900 mCi/μmol, 0.2 mL) with various doses of 1 (0 μg (vehicle), 0.02 mg/kg, 0.2 mg/kg, 1 mg/kg, and 3 mg/kg). Three animals per dose were used. 1 was dissolved in a vehicle (saline/alcohol (9:1) at pH 5.5). At 90 min after administration of the tracer, brain tissues were harvested, and their regional radioactivity content was determined. The dose-dependent blockade study of [¹⁸F]7c with 5 was done the same way.

### Blockade of [¹⁸F]7a with Nicotine and Cytisine

In vivo CB1 receptor blocking studies were carried out by subcutaneous (sc) administration of (−)-nicotine tartrate (5 mg/kg) or cytisine (1 mg/kg) followed by iv injection of the radiotracer [¹⁸F]7a (70 μCi, specific radioactivity of ˜14 000 mCi/μmol, 0.2 mL) 5 min thereafter. The drugs were dissolved in saline and administered in a volume of 0.1 mL. Control animals were injected with 0.1 mL of saline. At 90 min after administration of the tracer, brain tissues were harvested, and their radioactivity content was determined Blockade of [¹⁸F]7a with Non-α7-nAChR Drugs. In vivo receptor blocking studies were performed by administration of six drugs (Table 5), followed by iv injection of the radiotracer [¹⁸F]7a (70 μCi, specific radioactivity of approximately 14 000 mCi/μmol, 0.2 mL). The drugs (2 mg/kg, sc) were dissolved in a vehicle (saline/DMSO 5:1) and administered in a volume of 0.1 mL. Control animals were injected with 0.1 mL of the vehicle solution. At 90 min after administration of the tracer, brain tissues were harvested, and their radioactivity content was determined.

### Example 2

### Biodistribution Studies of [¹⁸F]7a and [¹⁸F]7c in Mice

### Baseline Studies in Mice

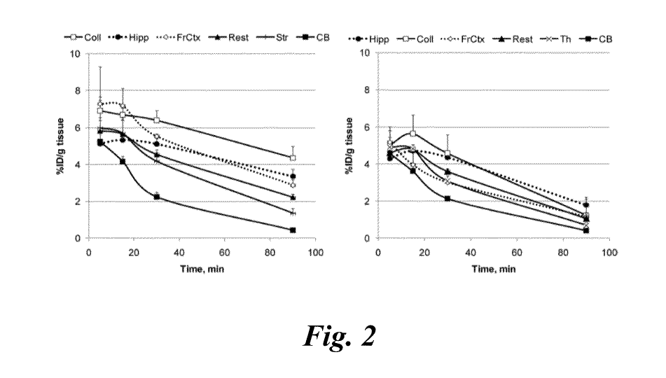

Radioligands [¹⁸F]7a and [¹⁸F]7c were evaluated in mice as potential PET tracers for imaging α7-nAChRs. After intravenous injection, [¹⁸F]7a and [¹⁸F]7c exhibited robust initial brain uptake followed by washout. The highest accumulation of radioactivity of both radioligands occurred in the superior/inferior colliculus, hippocampus, and frontal cortex. Moderate uptake was observed in thalamus and striatum, and the lowest radioactivity was seen in cerebellum (FIGS. 2-4). This distribution of radioactivity was similar to the previously published in vitro data on the distribution of α7-nAChRs in rodents. Clarke, et al., J. Neurosci. (1985); Whiteaker, et al., Eur. J. Neurosci. (1999).

The clearance rate of [¹⁸F]7a and [¹⁸F]7c from cerebellum was higher than that from any other region studied. The ratios of tissues to cerebellum increased steadily over the 90 min, reaching values of 10 for [¹⁸F]7a and 4.5 for [¹⁸F]7c. The better ratios for [¹⁸F]7a vs. [¹⁸F]7c are in agreement with in vitro α7-nAChR binding affinity of these compounds (Table 2).

### Specificity and Selectivity of [¹⁸F]7a and [¹⁸F]7c Binding in the Mouse Brain

A conventional in vivo blockade methodology with CNS drugs is used here for demonstration of specificity and selectivity at the α7-nAChR receptor in the mouse brain. A self-blockade study with a nonradioactive form of a radioligand estimates whether or not the binding is specific. A blockade study with a drug that is highly selective at the target binding site is expected to show the selectivity and specificity of the radioligand binding. A dose-escalation blockade with such a target selective drug provides further evidence of the radioligand specificity and selectivity, and it is useful for demonstration of the radioligand suitability for evaluation of conventional drug candidates. In addition, blockade with CNS drugs that do not bind at the target site provides more evidence of the radioligand selectivity versus other cerebral binding sites.

### Self-Blockade Studies

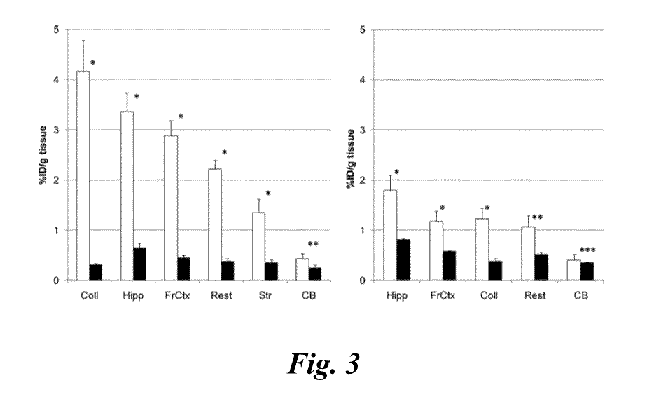

Self-blockade studies of [¹⁸F]7a with 7a (FIG. 3, left) and of [¹⁸F]7c with 7c (FIG. 3, right) demonstrated a reduction of the radioligand uptake in most brain regions except the cerebellum, a region with low density of α7-nAChRs. The studies showed that accumulation of [¹⁸F]7a and [¹⁸F]7c radioactivity in the mouse brain was specific. When the specific binding of the radioligands in the hippocampus and colliculus was estimated by using the radioactivity concentration in the blocked cerebellum as nonspecific binding, the specific binding value amounted to 94% and 80% and the baseline-to-blockade ratio in the α7-nAChR-rich regions was 13 and 5 for [¹⁸F]7a and [¹⁸F]7c, respectively. This result also demonstrated that [¹⁸F]7a exhibited a higher level of specificity and greater uptake in the mouse brain versus [¹⁸F]7c. Neither behavioral nor locomotor activity changes were observed in the mice in the blockade studies with 7a (0.3 mg/kg, iv) or 7c (0.2 mg/kg, iv).

### Blocking with Selective α7-nAChR Ligands

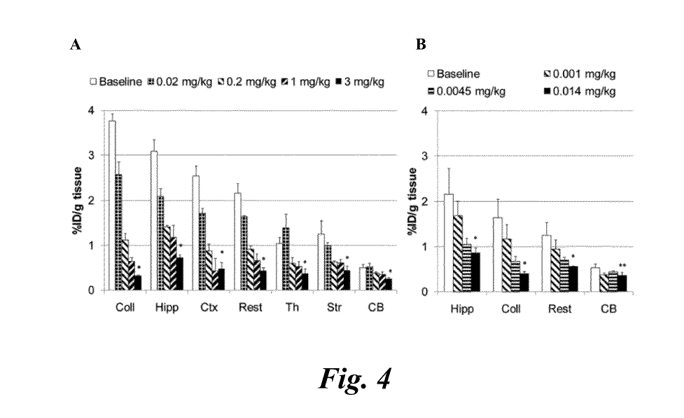

A blockade study of [¹⁸F]7a with 1, a selective α7-nAChR partial agonist with a Kᵢ of 22 nM, 58 showed a dose dependent blockade in all regions studied. However, in the α7-nAChR-poor cerebellum, the blockade was significant only with the highest dose of 1 (3 mg/kg) (FIG. 4, left). A similar dose-response study was performed with [¹⁸F]7c using compound 5, a selective α7-nAChR antagonist, as a blocker (FIG. 4, right). These studies confirmed that the in vivo binding of [¹⁸F]7a and [¹⁸F]7c was specific and mediated by α7-nAChR. The dose-escalation response demonstrated that both radioligands are suitable tools for evaluation of new α7-nAChR drug candidates. It is noteworthy that the doses of 1 that significantly blocked the [¹⁸F]7a binding in CD1 mice were comparable to the doses of 1 that significantly improved cognitive deficit in the various rodent models of schizophrenia. Hashimoto, et al., Biol. Psychiatry (2008); Pichat, et al., Neuropsychopharmacology (2007).

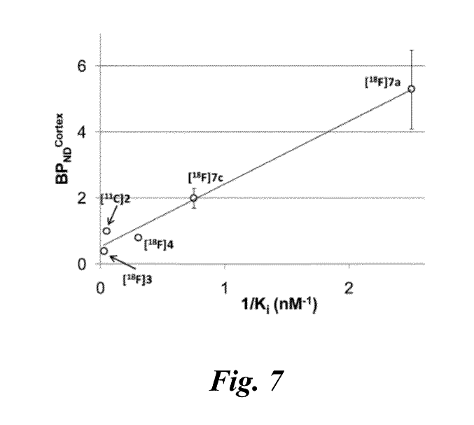

This finding suggests that [¹⁸F]7a is a suitable radioprobe for in vivo studies in mice with pharmacologically relevant doses of α7-nAChR drugs. Because the lowest regional uptake of [¹⁸F]7a and [¹⁸F]7c was seen in the cerebellum, the regional BP_{ND} values in mice were approximated for a single time point measurement (90 min) as BP_{ND}=(regional uptake/cerebellum uptake)−1 42 (Table 4). The substantially higher BP_{ND} values for [¹⁸F]7a are in agreement with greater binding affinity of this compound versus [¹⁸F]7c (Table 2; also see FIG. 7). The BP_{ND} values for both radioligands [¹⁸F]7a and [¹⁸F]7c were superior to all previously published α7-nAChR PET radioligands (Table 1).

TABLE 4
Approximate BP_{ND} Values (Unitless) of [¹⁸F]7a
and [¹⁸F]7c in the Mouse Brain Regions^{a}

| | region | | |
|---|---|---|---|
| compd | Coll | Hipp | Ctx |
| [¹⁸F]7a | 8.0 ± 1.6 | 5.5 ± 1.7 | 5.3 ± 1.2 |
| [¹⁸F]7c | 2.0 ± 0.5 | 3.1 ± 0.7 | 2.0 ± 0.3 |

^{a}Data: mean ± SD (n = 6).
Abbreviations: Coll, superior and inferior colliculus; Hipp, hippocampus; Ctx, cortex.

### Blocking with Nicotine and α4β2-nAChR Selective Cytisine

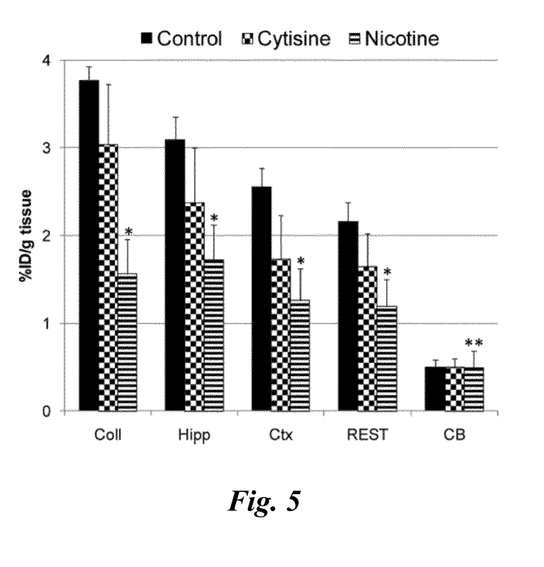

The blockade of [¹⁸F]7a in CD1 mouse brain with cytisine, a partial nicotinic agonist selective for α4β2-nAChR and other β2/β4-containing heteromeric nAChR subtypes while exhibiting low α7-nAChR binding affinity, 52, 55, 61 showed insignificant reduction of radioactivity accumulation in all regions studied (FIG. 5). This result demonstrated that [¹⁸F]7a manifested insignificant binding at α4β2-nAChRs in the mouse brain. The blockade study of [¹⁸F]7a with nicotine that binds at all nAChR subtypes including α7-nAChR52 showed significant blockade in all regions except the nAChR-poor cerebellum. This study suggests that [¹⁸F]7a can be used for nicotine addiction or smoking studies in mice. The lesser blockade of [¹⁸F]7a with nicotine (FIG. 5) in comparison with 1 (FIG. 4) is due to the rather modest binding affinity of nicotine at α7-nAChR (Kᵢ=610 nM).52

### Blocking with Non-α7-nAChR CNS Ligands

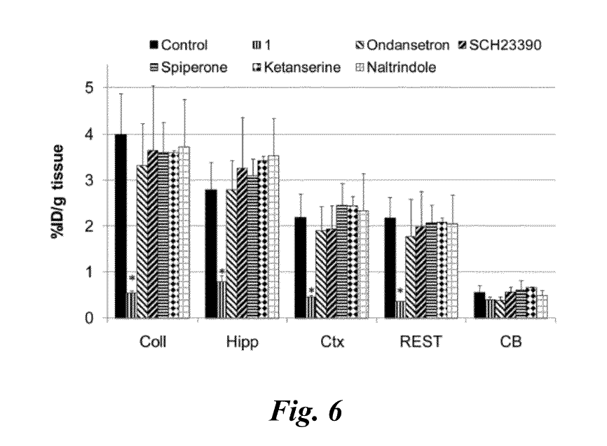

For determination of in vivo selectivity of [¹⁸F]7a for α7-nAChRs vs. several major CNS receptor systems, we compared the regional distribution (FIG. 6) of the radiotracer in control CD-1 mice vs. mice preinjected with various CNS active drugs or the positive control 1 (see Table 5 for the drug list). None of the drugs except 1 reduced accumulation of radioactivity when compared with controls (FIG. 6). The absence of blockade with the 5-HT3-selective drug ondansetron was especially remarkable because α7-nAChR ligands often bind to this receptor subtype. This finding suggests that in the mouse brain the radioligand [¹⁸F]7a was selective for α7-nAChRs versus several major cerebral binding sites.

TABLE 5
CNS Drugs (2 mg/kg, sc) for α7-nAChR
Selectivity Studies in Mice^{a}

| | | dose | time of administration |
|---|---|---|---|
| drug | target receptor | (mg/kg) | before radiotracer, min |
| 1 | selective α7-nAChR | 2 | 10 |
| | partial agonist | | |
| ondansetron | selective 5-HT₃ | 2 | 10 |
| | antagonist | | |
| SCH23390 | D₁- and D₅-antagonist | 2 | 10 |
| | and 5-HT_{1C/2C} | | |
| | agonist | | |
| spiperone | D₂-like and 5-HT_{2A} | 2 | 10 |
| | receptor antagonist | | |
| ketanserin | 5-HT₂/5-HT_{2C} | 2 | 10 |
| | antagonist | | |
| naltrindole | selective δ-opioid | 2 | 10 |
| | antagonist | | |

^{a}5-HT = 5-hydroxytryptoamine (serotonin).

### Comparison of Imaging Properties of [¹⁸F]7a and [¹⁸F]7c with Previous α7-nAChR PET Radioligands

Binding potential (BP_{ND}), a measure of in vivo specific binding and one of the most important imaging characteristics of a PET radioligand, is defined as the ratio of Bmax (receptor density) to KD (radioligand equilibrium dissociation constant) or the product of Bmax and binding affinity. Innis, et al., J. Cereb. Blood Flow Metab. (2007); Mintun, et al., Ann. Neurol. (1984).

Therefore, the binding affinities (1/Kᵢ) of α7-nAChR radioligands should correlate linearly with their BP_{ND} values. The comparison of all previously published α7-nAChR radioligands (Table 1) revealed little correlation between 1/Kᵢ and BP_{ND} (R²=0.05, not shown). It was likely that the lack of correlation was due to the wide variability in binding assay conditions for these compounds when performed by various research groups. When the α7-nAChR binding assay for the radioligands is performed under the same assay conditions (Tables 2 and 3), the binding affinities 1/Kᵢ correlate linearly (FIG. 7) with the cortical BP_{ND} values of [¹⁸F]7a and [¹⁸F]7c (Table 4) and [¹¹C]2, [¹⁸F]3, and [¹⁸F]4 (Table 1). Without wishing to be bound to any one particular theory, this finding may explain why the specific binding of the very high affinity radioligands [¹⁸F]7a and [¹⁸F]7c is superior to the previous α7-nAChR radioligands with lower binding affinities. This result emphasizes further the importance of high binding affinity for the imaging properties of α7-nAChR radioligands.

A series of 3-(1, 4-diazabicyclo[3.2.2]nonan-4-yl)dibenzo[b,d]-thiophene 5,5-dioxide derivatives with high binding affinities for α7-nAChRs (Kᵢ=0.4-20 nM) has been synthesized with potential application for PET imaging of α7-nAChRs. Two members of the series, 7a and 7c, with the best α7-nAChR binding affinities (Kᵢ of 0.4 and 1.3 nM, respectively) and high selectivity vs other nicotinic subtypes and 5-HT3, were radiolabeled with ¹⁸F. [¹⁸F]7a and [¹⁸F]7c readily entered the mouse brain and specifically and selectively labeled cerebral α7-nAChR receptors. The binding potential (BP_{ND}) values in mouse cortex of [¹⁸F]7a, [¹⁸F]7c, and previously published α7-nAChR radioligands correlated linearly with their binding affinities (1/Kᵢ) when the binding affinity values were determined under the same assay conditions. In agreement with the binding affinity of [¹⁸F]7a its BP_{ND} value in mice was substantially better than those of the previous α7-nAChR radioligands. The best PET radioligand of this new series [¹⁸F]7a exhibits excellent α7-nAChR imaging properties in the mouse brain. Therefore, [¹⁸F]7a holds promise as a highly specific PET radioligand for quantification of α7-nAChR receptors.

### Example 3

### Biodistribution Studies of [¹²⁵]14 in Mice

### Baseline Studies in Mice

Radioligands [¹²⁵I]14 was evaluated in mice as potential PET tracers for imaging α7-nAChRs. Mice received 2 μCi of [¹²⁵I]14 (specific radioactivity=1500 mCi/μmol) by tail vein injection. The regional distribution of the tracer in brain was assessed in the absence and presence of SSR180711, a selective partial agonist of α7-nAChR receptor. [¹²⁵I]14 showed a regional distribution similar to that of α7-nAChR (data not shown). At 180 min post injection the highest accumulation of ¹²⁵I radioactivity occurred in the superior colliculus (3.2% injected dose/g tissue (%1.D./g)), frontal cortex (2.74%1.D./g) and hippocampus (2.65%1.D./g) and lowest radioactivity occurred in the cerebellum (0.76%1.D./g). Regional brain distribution of [^{125I]}14 in CD-1 mice. A subcutaneous blocking dose of SSR180711 significantly inhibited [¹²⁵I]14 binding at 180 min after administration of the tracer in superior colliculus, but did not block in the cerebellum, a region with a low density of α7-nAChR (data not shown). This result demonstrated that [¹²⁵I]14 binding in the mouse brain is mediated with α7-nAChR.

### Example 4

### Biodistribution Studies of [¹⁸F]ASEM in Mice and Baboon

### In Vitro Inhibition Binding Assay of ASEM and Functional Electrophysiology Method

HEK293 cell culture and stable transfections of α7-nAChR and the ASEM inhibition binding assay with ¹²⁵I-a-bungarotoxin were performed as described previously. Xiao Y. et al. Acta Pharmacol. Sin. (2009). Whole-cell voltage clamp (holding potential, 270 mV) recordings from HEK293 cells stably transfecting the rat α7-nAChR were made with patch electrodes (5-6 MV) containing a solution (pH 7.2) composed of potassium gluconate (145 mM), ethylene glycol tetraacetic acid (5 mM), MgCl₂ (2.5 mM), 4-(2-hydroxyethyl)-1-piperazineethanesulfonic acid (HEPES) (10 mM), adenosine triphosphate sodium (ATP.Na) (5 mM), and guanosine triphosphate sodium (GTP.Na) (0.2 mM). Cells were continuously perfused with recording solution with the following composition: NaCl (130 mM), KCl (5 mM), CaCl₂ (2 mM), MgCl₂ (2 mM), glucose (10 mM), and HEPES (10 mM), pH 7.4, at a temperature of 24° C. The patch pipette was coupled to an amplifier (Axopatch 200B; Molecular Devices) and its signal filtered (5 kHz), digitized with a Digidata 1440A (Molecular Devices), and analyzed with pClamp 10 software (Molecular Devices). Acetylcholine was delivered to the cells rapidly by pressure application (picospritzer; World Precision Instruments) for 0.5 s. A bath was applied to the compound ASEM for 2 min before and during the application of acetylcholine by pressure application.

### Biodistribution Study in Mutant DISC1 and Control Mice

Male DISC1 (16-18 g) and control (17-19 g) mice both on a C57BL/6 background were generated as previously described (Pletnikov, M. V. et al. Mol. Psychiatry. (2008)) and were used for biodistribution studies, with 6 animals per data point. The animals were sacrificed by cervical dislocation at 90 min after injection of ¹⁸F-ASEM (2.6 MBq; specific radioactivity; 300 GBq/mmol, in 0.2 mL of saline) into a lateral tail vein. The brains were rapidly removed and dissected on ice. The brain regions of interest were weighed, and their radioactivity content was determined in an automated g counter with a counting error below 3%. Aliquots of the injectate were prepared as standards, and their radioactivity content was determined along with the tissue samples. The percentage injected dose per gram of tissue (% ID/g tissue) was calculated.

### Western Blot with DISC1 and Control Mice

Mice were euthanized at postnatal day 21 to evaluate the expression of α7-nAChR in mutant DISC1 and control animals. Frontal cortices were quickly dissected out on ice-cold phosphate-buffered saline and frozen on dry ice and kept at 280° C. until used. These samples were assayed for expression of mutant DISC1 Pletnikov, M. V. et al. Mol. Psychiatry. (2008). Membranes were incubated overnight at 4° C. with either mouse anti-myc antibody (Santa Cruz Biotechnology Inc.; 1:1,000) to assess the expression of mutant DISC1 tagged with myc or rabbit polyclonal antibody to α7-nAChR (ab10096 [Abcam Inc.]; 1:500). Secondary antibodies were peroxidase-conjugated goat antimouse (Kierkegaard Perry Labs; 1:1,000) or sheep antirabbit (GE Healthcare; 1:2,500). The optical density of protein bands on each digitized image was normalized to the optical density of b-tubulin as a loading control (Cell Signaling Technology Inc; 1:10,000). Normalized values were used for statistical analyses.

### Baboon PET Imaging and Baboon PET Data Analysis

PET experiments were performed on 3 male baboons (Papio anubis; weight, 20.1-26 kg) on the High Resolution Research Tomograph (CPS Innovations, Inc.). The animals were anesthetized and handled as described previously (data not shown). Kuwabara H, et al. J. Nucl. Med. (2012). Three animals were scanned with ¹⁸F-ASEM in baseline scans. Dynamic PET images were acquired in a 3-dimensional list-mode for 90 min after an intravenous bolus injection of ¹⁸F-ASEM (246-319 MBq; n=3), with specific radioactivities in the range of 343-1,764 GBq/mmol. In 2 blocking scans, the blocker SSR180711 solution in saline was given as intravenous bolus doses (0.5 or 5 mg/kg) 90 min before the radioligand ¹⁸F-ASEM injection (doses, 147 and 251 MBq; specific radioactivity, 462 and 1,014 GBq/mmol). The blocking scans were obtained for 1 of the baboons that were used in the baseline scans and separated at least 32 d from each other and the baseline scan. A locally developed volume-of-interest (VOI) template was transferred to each animal's MR image using spatial normalization parameters given by SPM5 (statistical parametric mapping. Ashburner J, et al. Academic Press (2004); available at http://www.fil.ion.ucl.ac.uk/spm/software/spm5) and adjusted for anatomic details. Then, VOIs were transferred to the PET spaces of the baseline and blocking scans using the MR imaging-to-PET coregistration module of SPM5. Ashburner J, et al. Academic Press (2004). Time-radioactivity curves (time-activity curves) of regions were obtained by applying the VOIs on PET frames. One- and 2-tissue-compartmental models (TTCM) were used for derivation of regional distribution volume (V_{T}) for ¹⁸F-ASEM, with and without setting the K₁-k₂ ratio to the estimate of a large region (denoted as TTCM-C). Akaike information criteria (Akaike H. IEEE Trans. Automat. Contr. 1974) and the numbers of outliers were used to identify the optimal plasma input method for the radioligand.

In addition, the plasma reference graphical analysis (PRGA) was evaluated. Logan J. et al. J. Cereb. Blood Flow Metab. (1990). In blocking scans, occupancies of α7-nAChRs by SSR180711 were obtained as follows: occupancy ΔV_{T}/(V_{T}[baseline]−V_{ND}), where ΔV_{T} was given by V_{T}(baseline)−V_{T}(blocking), and V_{ND}, the distribution volume of nondisplaceable radioligand, was obtained as the x-intercept of the Lassen plot (Lassen N A, et al. J. Cereb. Blood Flow Metab. (1995)) of ΔV_{T} (=y) versus baseline V_{T}.

### ¹⁸F-ASEM: Radiometabolite Analysis in Baboon and Mice

Baboon arterial blood samples were withdrawn at 5, 10, 20, 30, 60, and 90 min after ¹⁸F-ASEM injection, and plasma was analyzed by HPLC. Male CD-1 mice (25-26 g) were injected via the lateral tail veins with 37 MBq of high-specific-activity ¹⁸F-ASEM. The mice were killed by cervical dislocation at 2 and 30 min after injection, and a terminal blood sample was obtained. The mouse brains were rapidly removed and analyzed by HPLC (data not shown).

### Binding Affinity

In 2 experiments, unlabeled ASEM exhibited high in vitro binding affinity to HEK293 cells stably transfected with rat α7-nAChR (Kᵢ 5 0.3, 0.3 nM) (¹²⁵I-a-bungarotoxin).

### In Vitro Functional Assay

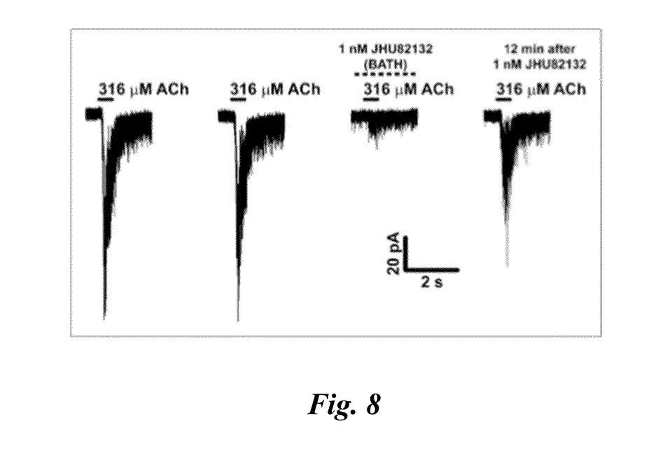

The functional activity of unlabeled ASEM was examined using whole-cell voltage clamp measurements in HEK293 cells expressing α7-nAChRs. As shown in FIG. 8, acetylcholine at a concentration of 316 mM activates these receptors, and ASEM at a concentration of 1 nM nearly completely blocked activation by acetylcholine. Moreover, a partial block persists during the short period of washing, probably because of the high affinity of ASEM.

### Brain Distribution of ¹⁸F-ASEM in Mutant DISC1 and Control Mice

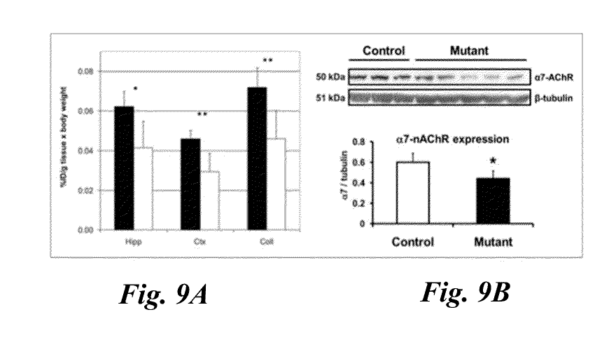

Mutant DISC1 mice provide a model for brain and behavioral phenotypes seen in schizophrenia. Pletnikov, M. V. et al. Mol. Psychiatry. (2008). The comparison of regional brain uptake of ¹⁸F-ASEM in mutant DISC1 versus control mice demonstrated that the uptake in the mutant mice was significantly lower in all regions studied. Because of the difference in the mouse weight (up to 15%), the uptake values were corrected for the body weight (% ID/g tissue·body weight) (FIG. 9A). Western blot analysis of the expression of α7-nAChR in the cortical regions was in agreement with the biodistribution of ¹⁸F-ASEM. A significant decrease in the levels of the receptor in the cortex of mutant DISC1 mice, compared with control mice was found (FIG. 9B).

### PET Imaging in Papio Anubis Baboons

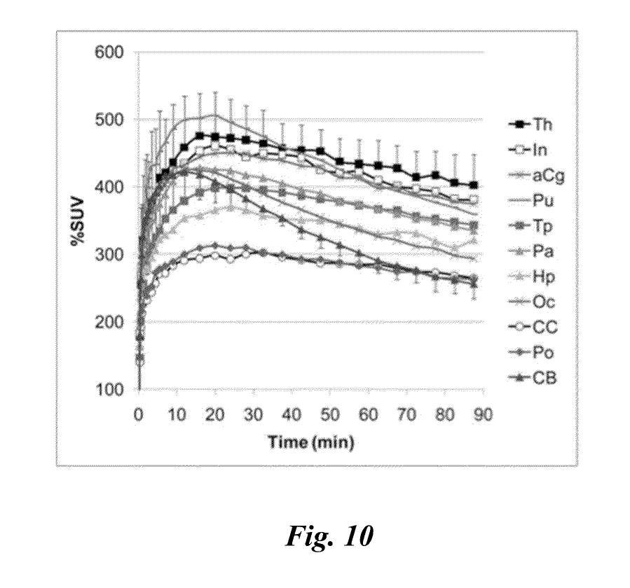

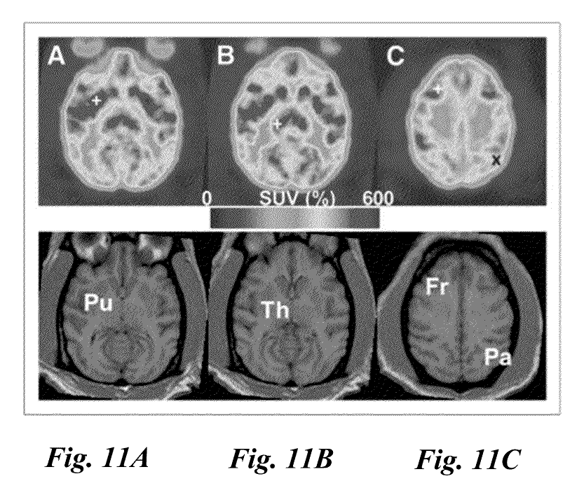

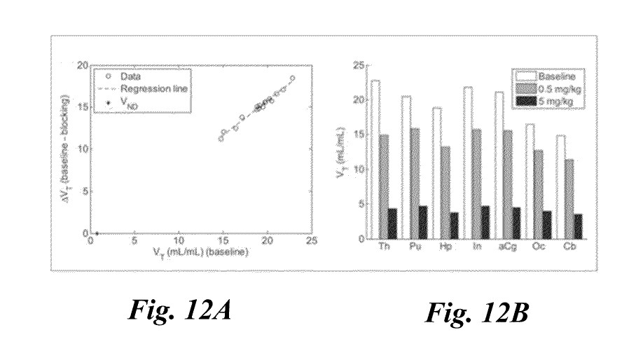

Heterogeneous uptake of radioactivity into the baboon brain was observed in baseline experiments after bolus injection of ¹⁸F-ASEM in 3 baboons as shown in FIGS. 10 and 11. The highest accumulation of radioactivity occurred in the thalamus, insula, and anterior cingulate cortex. The intermediate uptake was observed in the putamen, hippocampus, and several cortical regions. The lowest uptake was in the corpus callosum, pons, and cerebellum. The time-activity curves of the cerebellum peaked before 20 min and decreased rapidly, whereas time-activity curves of other regions were slower with wider peaks and decreased relatively slowly (FIG. 10). In the 3 baseline experiments, no blocking effect was observed due to the variation of ¹⁸F-ASEM specific activity from high (343 GBq/μmol) to very high (1,764 GBq/μmol). The kinetics of ¹⁸F-ASEM in the brain fitted well to a TTCM. The PRGA plots reached an asymptote (the coefficient of determination, R²>0.995) at 30 min in all regions. Therefore, PRGA was used for further analyses. Regional values of V_{T} of ¹⁸F-ASEM in baboon are shown in FIG. 12B. The thalamus, insula, and anterior cingulate cortex provided the highest VT values, and the pons, corpus callosum, and cerebellum showed the lowest VT values. Injection of SSR180771, a selective α7-nAChR partial agonist (Kᵢ=22 nM), reduced the regional uptake of ¹⁸F-ASEM in the baboon brain in a dose-dependent manner (FIG. 7). Regional V_{T} values in baseline and blockade experiments are shown in FIG. 6.

Lassen plots showed a linear arrangement for 0.5 and 5 mg/kg doses, as exemplified for the dose of 5 mg/kg in FIG. 12A (for a dose of 0.5 mg/kg, ΔV_{T}=0.39V_{T}−2.1; R²=0.643; V_{ND}=5.4 mL/mL) Mean occupancy values increased from 38% with a dose of 0.5 mg/kg to 80.5% with a dose of 5 mg/kg using individual V_{ND} values, and from 32.9% to 94.1% using the mean V_{ND} value of 2 doses. Although estimates of V_{ND} differed between 2 blocking scans, individual values were several folds lower than the lowest observed V_{T} (14 mL/mL in the pons) among the tested regions. This finding confirmed the lack of α7-nAChR-free regions in the baboon brain and low nonspecific binding of ¹⁸F-ASEM across regions (e.g., less than 30% in the pons and cerebellum and lower in other regions) and explained consistent occupancy estimates. Regional BP_{ND} ([V_{T}/V_{ND}]−1) values of ¹⁸F-ASEM in the baboon brain ranged from 3.9 to 6.6 (unitless), using the mean V_{ND} value of the 2 blocking scans.

### Metabolism of ¹⁸F-ASEM in Mouse and Baboon

Radiometabolite analysis of blood samples from CD-1 mice and baboons by reversed-phase HPLC showed that the parent compound ¹⁸F-ASEM was metabolized to 2 major hydrophilic species. The combined radiometabolites in the plasma reached values of 70% in baboons and approximately 99% in mice at 90 and 30 min after injection, respectively. These radiometabolites do not enter the brain to an appreciable extent, because at least 95% of the unchanged parent ¹⁸F-ASEM was present in the mouse brain versus approximately 1% in the mouse blood after intravenous administration of ¹⁸F-ASEM. The amount of unchanged parent ¹⁸F-ASEM in the baboon brain should be even greater than that in mouse (0.95%) because the metabolism in baboon is slower. This observation suggests that modeling of the metabolites may not be necessary for quantification of α7-nAChR with ¹⁸F-ASEM.

In vitro binding assay studies have demonstrated that ASEM exhibits high α7-nAChR binding affinity in rat brain membranes and excellent selectivity versus other heteromeric nAChR subtypes and 5-HT₃. Gao, Y. et al. J. Med. Chem. (2013). Those studies demonstrated that ASEM exhibits at least an order of magnitude greater binding affinity than previous α7-nAChR PET radioligands. Gao, Y. et al. J. Med. Chem. (2013). The high α7-nAChR binding affinity of ASEM in the binding assay with the HEK293 cell line expressing rat α7-nAChR (Kᵢ=0.3 nM) has been reported. The functional assay demonstrated that ASEM is a powerful antagonist of α7-nAChR, as disclosed in FIG. 10, which is in accord with functional properties of des-fluoro-ASEM, 3-(1,4-diazabicyclo[3.2.2]nonan-4-yl)dibenzo[b,d]thiophene 5,5-dioxide, which was recently published by Abbott Labs. Schrimpf, M. R. et al. Bioorg. Med. Chem. Let. (2012). This functional property may also be advantageous from the standpoint of safety if ¹⁸FASEM is used in human PET studies because it should not cause toxic effects that are common among nicotinic agonists. Biton, B. Neuropsychopharmacology (2007).

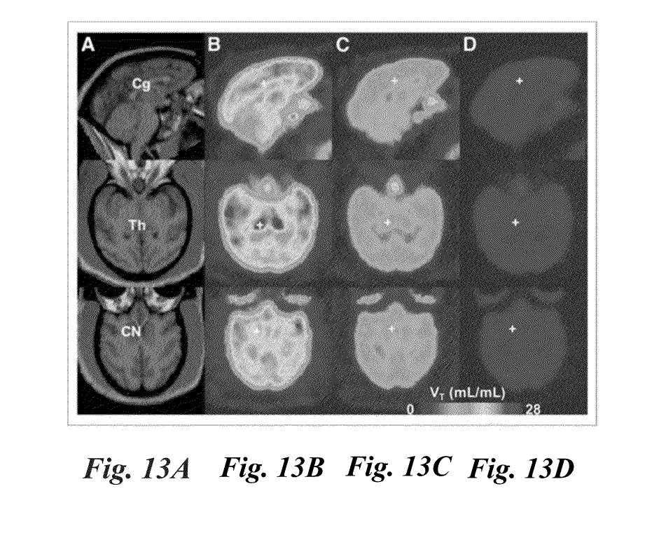

The initial in vivo distribution studies in control mice have demonstrated that ¹⁸F-ASEM selectively labels α7-nAChR with very high specificity (BPm=8). Gao, Y. et al. J. Med. Chem. (2013). On the basis of the favorable imaging properties identified in normal mice, we investigated ¹⁸F-ASEM cerebral binding in mutant DISC1 mice, a rodent model of schizophrenia. Pletnikov, M. V. et al. Mol. Psychatry (2008). Previous postmortem research demonstrated significantly lower densities of α7-nAChR in the cortical and subcortical (hippocampus) brain regions of schizophrenic subjects versus controls. Thomsen, M. S. Curr. Pharm. Des. (2010). In agreement with this in vitro human data, the brain regional distribution experiments with DISC1 mice showed a significant reduction of ¹⁸F-ASEM binding in the α7-nAChR-rich colliculus, cortex, and hippocampus in comparison with control animals (FIG. 9A). Western blot data (FIG. 9B) of α7-nAChR protein expression in the cortex of DISC1 and control animals was in agreement with ¹⁸F-ASEM binding. This result in DISC1 mice is consistent with previous postmortem brain studies of subjects with schizophrenia (Thomsen, M. S. Curr. Pharm. Des. (2010)) and further emphasizes the potential utility of this new radioligand for imaging α7-nAChR in disease. ¹⁸F-ASEM exhibited high (500% standardized uptake value [SUV]) and reversible brain uptake in baboon brain experiments (FIGS. 4 and 5). The cerebral α7-nAChR is heterogeneously distributed in the primate brain, with the highest concentration in the thalamus, putamen, several cortical regions, and hippocampus. Kulak, J. M., et al. Brain Res. (2004); Kulak, J. M. et al. Eur. J. Neurosci. (2006); Breese, C. R. et al. J. Comp. Neurol. (1997); Han, Z. Y., J. Comp. Neurol. (2003). The observed PET regional distribution patterns of ¹⁸F-ASEM in the baboon brain (thalamus.putamen, cortex, hippocampus, caudate nucleus, globus pallidus.corpus callosum) are consistent with in vitro data in rhesus and cynomolgus macaque monkeys. Kulak, J. M., et al. Brain Res. (2004); Kulak, J. M., et al. Eur. J. Neurosci. (2006); Han, Z. Y., J. Comp. Neurol. (2003). The existing quantitative nonhuman primate data describing the brain distribution of α7-nAChR using in vitro autoradiography are detailed only for subcortical regions but limited for cortical regions or semiquantitative. Kulak, J. M., et al. Brain Res. (2004); Kulak, J. M., et al. Eur. J. Neurosci. (2006); Han, Z. Y., J. Comp. Neurol. (2003). The PET ¹⁸F-ASEM baboon experiments demonstrated that the lowest α7-nAChR uptake, albeit still considerable, was in the cerebellum. The cerebellum was not assessed in the previous monkey autoradiography studies. Kulak, J. M., et al. Brain Res. (2004); Kulak, J. M., et al. Eur. J. Neurosci. (2006); Han, Z. Y., J. Comp. Neurol. (2003). It is noteworthy that the uptake of radioactivity in the baboon skull was low, suggesting little metabolism of ¹⁸F-ASEM to ¹⁸F-fluoride that can confound PET studies with ¹⁸F-labeled agents. The dose-dependent blockade of ¹⁸F-ASEM with the selective α7-nAChR partial agonist SSR180711 (FIGS. 12 and 13) demonstrated that the binding of the radioligand in the baboon brain was specific (up to 80%-90%) and mediated by α7-nAChR. The level of specific binding of ¹⁸F-ASEM is well above the conventional minimum of the required specific binding value ($50%) for a clinically viable PET radioligand. ¹⁸F-ASEM is suitable for quantitative analysis, and its BP_{ND} values (3.9-6.6) in the baboon brain are rather high. For comparison, the BP_{ND} values of all previously published α7-nAChR radioligands did not exceed 1. Horti, A. G., et al. Curr. Pharm. Des. (2006); Toyohara, J., et al. Curr. Top Med. Chem. (2010); Brust, P. et al. InTech. (2012); Gao, Y., et al. J. Med. Chem. (2013). This high specific binding of ¹⁸F-ASEM in combination with high brain uptake and V_{T} values, reversible brain kinetics, and absence of active metabolites make this radioligand an excellent candidate for further translation to human PET imaging of α7-nAChRs.

### Example 5

### Biodistribution Studies of [¹⁸F]ASEM in Human

### PET Imaging Procedures

Subjects were instructed not to ingest any alcohol, drugs, or over-the-counter medications for 48 h prior to PET scans and to arrive at JHU PET Center approximately 2-3 h before the scheduled first tracer injection time. Laboratory studies upon arrival included a urine toxicology screen, alcohol breathalyzer test, urine cotinine test, hematology, chemistry panel, and urine pregnancy screen for women. PET studies were performed on the high resolution research tomograph (HRRT) (Siemens)—the highest resolution (<2 mm) commercially available dedicated human brain PET scanner. A radial arterial catheter was used to obtain samples for plasma radioactivity for the kinetic model input function. An intravenous catheter was inserted into the antecubital vein for blood sampling and ligand injection. Each subject was fitted with a thermoplastic mask modeled to his or her face to reduce head motion during the PET study. A 6-min attenuation scan was performed using a rotating Cs-137 point source. Each subject was carefully monitored for subjective symptoms throughout the procedure. Vital signs were obtained pre-injection and at 15, 30, 60, 90, and 120 min post-injection. A 3-lead ECG was performed throughout the scan, with 12-lead ECG obtained pre-injection and at 90 min post-injection after scanning was completed. The emission scan began with a bolus (about 1 min) injection of [¹⁸F]ASEM and lasted 90 min in a 3-D list mode. Five male subjects were injected with 13.9-16.2 mCi (15.1±6.7 mCi; mean±SEM) with a mass ASEM dose of 0.20-0.67 mcg (0.35±0.15 mcg; mean±SEM) and specific activity of 8,000-27,300 mCi/μmol (18,600±8,300 mCi/μmol; mean±SEM). Arterial blood samples were obtained throughout the 90-min scan (approximately every 5 s initially and increasing to every 5 min after 30 min) Samples were centrifuged at 1,200×g, and the radioactivity in plasma was measured with a cross-calibrated gamma counter. Selected plasma samples (0, 2, 5, 10, 20, 30, 45, 60, and 90 min samples) were analyzed with high pressure liquid chromatography (HPLC) for radioactive metabolites in plasma, as described previously for baboon studies. Horti, A. G., et al. J. Nucl. Med. (2014). Reconstruction of Emission Scan PET images were reconstructed in list mode using the iterative ordered subset expectation-maximization (OSEM) algorithm with 6 iterations, 16 subsets, data-mashing (span) of 3, and maximum ring difference of 67 and correcting for attenuation, scatter, and deadtime. The following frame sequence was used: four 15-s, four 30-s, three 1-min, two 2-min, five 4-min, and twelve 5-min frames or a total of 30 frames for the 90-min scan. The radioactivity was corrected for physical decay to the injection time. Each PET frame consists of 256 (left-to-right) by 256 (nasion-to-inion) by 207 (neck-to-cranium) voxels.

### MR Imaging Procedures

Structural magnetic resonance (MR) of the brain was obtained to define volumes of interest (VOIs) and for gray and white matter segmentation. All MR imaging was done on the Siemens 3T TRIO at the B17 software level.

### PET Data Analysis

VOIs VOIs were defined automatically on individual subjects' SPGR MRI volumes using FSL's (The FMRIB Software Library Jenkinson, M., et al. Neurimage (2012) FIRST tool (Patenaude, B., et al. Neuroimage (2011) for subcortical regions and the Freesurfer tool (Fischl, b., et al. Creb cortex (2001) for cortical regions. Those automated VOIs were manually edited to fit the structures of interest using a locally developed VOI tool (VOILand). Refined VOIs were transferred from MRI to PET spaces according to MRI to PET coregistration parameters that were obtained by the co-registration module of SPM12 (Ashburner, J., et al. Human Brain Function (2004)). The VOIs in PET space were applied to PET frames to obtain time-activity curves (TACs) of various brain regions. Head motion correction (HMC) was performed using the coregistration module of SPM12 and/or the HRRT reconstruction head movement correction algorithm (Keller, S. H., et al. J. Nucl. Med. (2012)). Derivation of the Outcome Variable, Distribution Volume (V_{T}), and Binding Potential (BPND) Using Human Reference Tissue (see below) Standard compartmental models including one tissue (OTCM) and two tissue without (TTCM) and with (TTCMC) constraining the K₁/k₂ ratio (K₁ and k₂ are blood-brain and fractional brain-blood clearance constants) to the observed value of a low-receptor region were tested. Non-compartmental plasma reference graphical analysis (PRGA (Logan, J., et al. J. Cereb. Blood Flow Metab. (1996)) was tested for whether the kinetic behavior of [¹⁸F]ASEM follows underlying assumptions of this model for radioligands with measurable dissociation (i.e., PRGA plots of region reach asymptotes sometime after the tracer injection, often denoted as t*) within 10 min of the radiotracer injection). In these analyses, metabolite-corrected plasma TACs were obtained by applying the metabolite-corrected input function given by HPLC analysis to total plasma TACs after interpolating at plasma sample times using the piecewise cubic Hermite interpolation implemented in MATLAB (Cambridge, Mass., USA).

### Human PET Studies

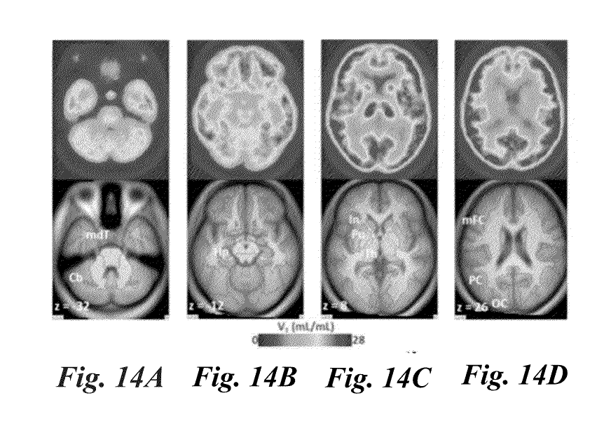

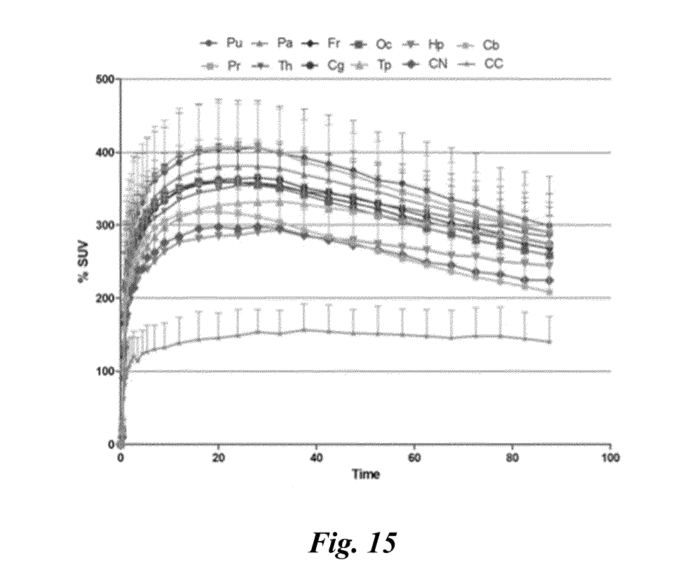

AS disclosed by FIG. 14A-FIG. 14D [¹⁸F]ASEM readily entered the human brain after a bolus injection and demonstrated reversible kinetics with a peak (% SUV=400) at 10-15 min (FIG. 15). The regional brain distribution of [¹⁸F]ASEM was comparable to the post-mortem data in the human brain [Court J A, Martin-Ruiz C, Graham A, Perry E (2000) Nicotinic receptors in human brain: topography and pathology. J Chem Neuroanat 20:281-298; Breese C R, Adams C, Logel J et al (1997).

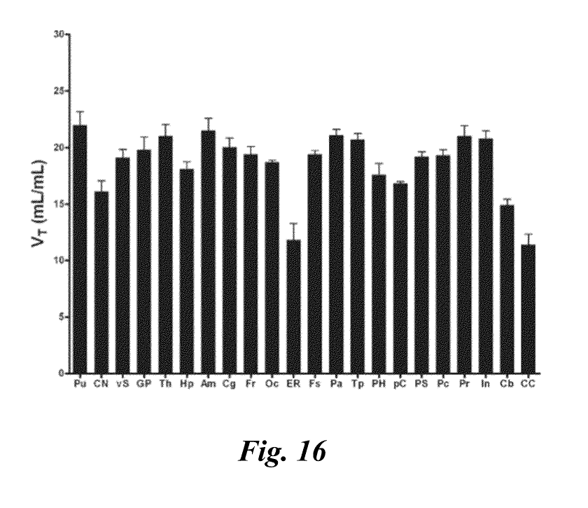

Comparison of the regional expression of nicotinic acetylcholine receptor alpha7 mRNA and [125I]-alpha-bungarotoxin binding in human postmortem brain. J Comp Neurol] and was similar to the distribution of [¹⁸F]ASEM in the baboon brain [Horti A G, Gao Y, Kuwabara H et al (2014) 18F-ASEM, a radiolabeled antagonist for imaging the alpha7-Nicotinic acetylcholine receptor with PET. J Nucl Med]. The OTCM, TTCM, and TTCMC fit observed tissue and plasma TACs sufficiently well without showing systematic deviations of normalized residues (the residue over observed radioactivity averaged across subjects G5%) at individual frames in all regions. Akaike information criterion values were not different among the three methods (tG0.67; p90.68), indicating that the goodness of fits were statistically indistinguishable when differences in numbers of parameters were taken into consideration. Using all frames (0-90 min), V_{T} values of the three methods correlated well (OTCM=0.92·TTCM+1.89; R²=0.878; TTCMC=1.0·TTCM−0.6; R²=0.910) excluding one outlier (V_{T}=60 ml/ml) observed with TTCM. Estimates of V_{T} were stable after 60 min (R290.827; 0-60 versus 0-90 min) in the three methods excluding the outlier. Altogether (no outliers and a better time consistency), TTCMC appeared to be the most appropriate among compartmental models. PRGA plots reached asymptotes by 10 min in all regions (R29 0.97) as we observed in our pre-clinical study in the baboon brain [Horti A G, Gao Y, Kuwabara H et al (2014) 18F-ASEM, a radiolabeled antagonist for imaging the alpha7-Nicotinic acetylcholine receptor with PET. J Nucl Med]. Estimates of V_{T} were stable after 60 min (V_{T}[60 min]=0.98·V_{T}[90 min]+0.05; R²=0.969). Showing a better time consistency, PRGA appeared to be appropriate for [¹⁸F]ASEM over compartmental models and was used for these results. At present, it is not clear whether a reference region (i.e., receptor free) exists for α7-nAChRs. White matter regions such as corpus callosum (CC) showed the lowest accumulation of [¹⁸F]ASEM. If we use the CC as a reference tissue region, BPND [0. Innis R B, Cunningham V J, Delforge J et al (2007) Consensus nomenclature for in vivo imaging of reversibly binding radioligands. J Cereb Blood Flow Metab 27:1533-1539] may be obtained by the (target V_{T}/reference V_{T})−1. Precuneus, parietal, occipital, and cingulate cortices and putamen showed relatively high values of V_{T} (920 ml/ml) and binding potential (BPND-1) while entorhinal cortex, cerebellum, caudate, and CC showed lower values of V_{T} (G15 ml/ml) (FIG. 16). The test-retest variability (TRV) averaged at 10.8±5.1% for medium and high V_{T} regions for the two subjects which were completed with two scans separated by a few days.

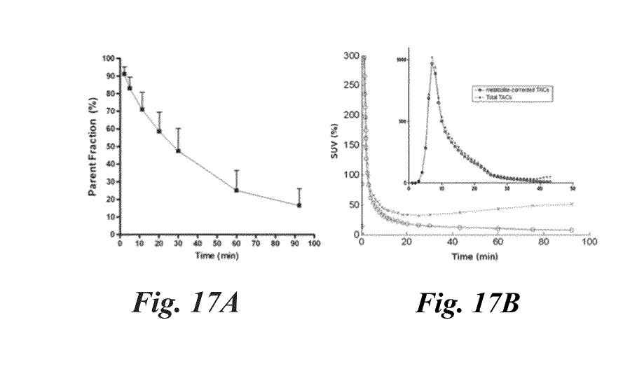

### [¹⁸F]ASEM Metabolite Analysis in Human Plasma

[¹⁸F]ASEM was metabolized in the body to polar radiometabolites at rates comparable to other PET radioligands for CNS receptors. Reverse phase HPLC analysis demonstrated that all human [¹⁸F]ASEM radiometabolites were the same as those in baboon plasma. Horti, A. G., et al. J. Nucl. Med. (2014). At 30 min and at 90 min, 52.6±12.9 and 83.5±9.7%, respectively, of parent [¹⁸F]ASEM was metabolized (FIG. 17A). Plasma TACs peaked within 1 min as disclosed in FIG. 17B. Thereafter, metabolite-corrected TACs declined mono-exponentially while total TACs started to increase gradually after 20 min, suggesting initial distribution of [¹⁸F]ASEM to various organs and subsequent re-entry of its metabolites to the circulation.

### Mouse Biodistribution Studies with Blockade Using Human Equivalent Doses of DMXB-A (GTS-21)

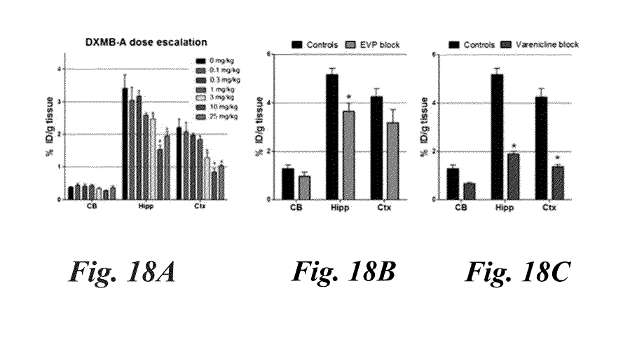

AS disclosed by FIG. 18A, [¹⁸F]ASEM binding in the α7-nAChR-rich brain regions was blocked in a dose-dependent fashion by DMXB-A. The blockade was significant at a mouse-equivalent dose [Reagan-Shaw S, Nihal M, Ahmad N (2008) Dose translation from animal to human studies revisited. FASEB J 22:659-661] comparable to the clinical dose of DMXB-A (25 mg/kg) and two lower doses (3 and 10 mg/kg), but it was not significant at the lowest doses (0.1-1 mg/kg). Specifically, at a dose of 25 mg/kg, DMXB-A significantly blocked [¹⁸F]ASEM binding by 50-60% in the hippocampus, cortex, and superior and inferior colliculus (p<0.01). The lower dose of 10 mg/kg showed similar levels (50-70%) of blockade in the hippocampus, cortex, and subcolliculus (p<0.01). At the dose of 3 mg/kg, the observed blockade was smaller-28% in the hippocampus (p<0.05) and 40% in the cortex (p<0.05). The lowest doses of 0.1, 0.3, and 1 mg/kg did not show significant blockade. As disclosed FIG. 18B and FIG. 18C, similar significant blockade of [¹⁸F]ASEM was observed with two other nicotinic drugs in clinical trials that bind to the α7-nAChR, EVP-6124 [Prickaerts, J., et al (2012), and varenicline. Rollema, H., et al. J. Pharmacol. (2010). Both EVP-6124 and varenicline at a dose of 0.18 mg/kg (equivalent to the clinical dose of 1 mg/kg) blocked ASEM binding by 40-60% in the hippocampus and cortex (p<0.05).

In vitro [¹⁸F]ASEM selectively binds at α7-nAChR with subnanomolar binding affinity (rat Ki=0.4 nM; human Ki=0.3 nM) that is one to two orders of magnitude better than those of the previous best α7-nAChR PET tracers ([¹¹C]NS14492, [¹¹C]NS10743, and [¹⁸F]AZ11637326). Gao, Y., et al. J. Med. Chem. (2013). In addition, the α7-nAChR inhibition binding affinity of [¹⁸F]ASEM is substantially better than that of its structural para-isomer 4-(8-[¹⁸F]fluorodibenzo[b,d]thiophen-3-yl)-1,4-diazabicyclo[3.2.2]nonane 5,5-dioxide [¹⁸F]para-ASEM (Ki=1.3 nM). Gao, Y., et al. J. Med. Chem. (2013). After the original publication of [¹⁸F]para-ASEM, Gao, Y., et al. J. Med. Chem. (2013), this poorer affinity ligand was selected by others under a different name, Kranz, M., et al. J. Nucl. Med. (2014), as a potential PET tracer despite its less than optimal properties.

In vivo studies showed that [¹⁸F]ASEM readily entered the mouse and baboon brains and specifically and selectively labeled cerebral α7-nAChR receptors (2014). The binding potential BPND values of [¹⁸F]ASEM in the mouse brain regions rich in α7-nAChR such as the cortex, hippocampus, and colliculus were BPND=5.3, 5.5, and 8.0, respectively. In the baboon brain, [¹⁸F]ASEM exhibited BPND values of 3.9-6.6. Horti, A. G., et al. J. Nucl. Med. (2014). The BPND values for [¹⁸F]ASEM were at least 10 times greater than those of all previously published α7-nAChR PET radioligands. Gao, Y., et al. J. Med. Chem. (2013). Thus, the initial human PET studies provide evidence of the great potential for this radiotracer to image both α7-nAChR decrements (as expected in SCZ, traumatic brain injury, and Alzheimer's disease) and increases (as may occur in bipolar disorder).

### Potential for [18F]ASEM Occupancy Studies

The critical role of the α7-nAChR in human physiology has recently been supported by clinical studies with α7-nAChR agonists—emerging drugs for treatment of cognitive dysfunction. Olincy, A., et al. Handb. Exp. Pharmacol. (2012); Mazurov, A. A., et al. J. Med. Chem. (2011). Several drugs that target α7-nAChRs are now in the various clinical phases of development for numerous pathologies. Taly, A. et al. Curr. Drug Targets (2012); Wallace, T. L., et al. Expert Opin. Ther. Targets (2013). Dimethoxybenzylidene anabaseine (DMXB-A or GTS-21) was the first selective α7-nAChR agonist that demonstrated cognitive enhancement in patients with SCZ. Freedman, R., et al. Am. J. Psychiatry (2008); Olincy, A., et al Arch. Gen. Psychiatry (2006). Currently, DMXB-A is in clinical trials for treatment of SCZ and other disorders.

### Example 6

### Summary and Discussion

In summary, several PET radioligands were evaluated. While all show some α7-nAChR inhibition, [¹⁸F]ASEM exhibits excellent α7-nAChR imaging properties in the mouse brain. A SPECT radioligand [¹²⁵I]14 also was evaluated, and it binds with high affinity at α7-nAChR and exhibits low binding affinity at other nAChR subtypes. Therefore, [¹²⁵I]14 holds promise as a specific SPECT radioligand for quantification of α7-nAChR receptors.

Previous rodent biodistribution studies used SSR180711, which successfully blocked [¹⁸F]ASEM binding in both mouse and baboon brain, but clinical trials of SSR180711 were terminated in part due to insufficient efficacy and unacceptable side effects. Evidence for blockade in the mice brain with DMXB-A and measurable blockade with the α7-nAChR partial agonist EVP-6124, Prickaerts, J., et al. Neuropharmacology (2012), and varenicline, which binds at two main CNS nAChR subtypes, α7-nAChR and α4β2, Rollema, H., et al Br. J. Pharmacol. (2010), have been presented. Both EVP-6124, Prickaerts, J., et al. Neuropharmacology (2012), and varenicline are currently and have been previously used in clinical trials. This demonstrates the definitive feasibility of [¹⁸F]ASEM for human α7-nAChR target engagement (by measuring the degree of receptor occupancy) to facilitate treatment strategies and opens new horizons for studying the biochemical mechanism of drugs for treatment of cognitive deficits in patients with SCZ. [¹⁸F]ASEM has enabled the first successful human PET studies of the α7-nAChR. The studies show suitable brain uptake with an appropriate regional distribution, matching the post-mortem results, and high, reversible binding sufficient for interrogating neuropsychiatric disorders in vivo. The in vivo rodent studies demonstrate the feasibility to measure receptor occupancy (and have target engagement) of clinical α7-nAChR drugs in a dose-dependent manner.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Akaike H. A new look at statistical model identification. IEEE Trans Automat Contr. 1974; 19:716-722.
Albuquerque, E. X.; Pereira, E. F.; Alkondon, M.; Rogers, S. W. Mammalian nicotinic acetylcholine receptors: from structure to function. Physiol. Rev. 2009, 89, 73-120.
Anderson, D. J.; Bunnelle, W.; Surber, B.; Du, J.; Surowy, c.; Tribollet, E.; Marguerat, A.; Bertrand, D.; Gopalakrishnan, M. [3H]A-585539 [(1S,4S)-2,2-dimethyl-5-(6-phenylpyridazin-3-yl)-5-aza-2-azoniabicyclo[2.2.1Jheptane], a novel high-affinity alpha7 neuronal nicotinic receptor agonist: radioligand binding characterization to rat and human brain. J Pharmacol Exp Ther 2008, 324, 179-187.
Auld D S, Kornecook T J, Bastianetto S, Quirion R. Alzheimer's disease and the basal forebrain cholinergic system: relations to beta-amyloid peptides, cognition, and treatment strategies. Prog Neurobiol. 2002; 68:209-245. 7
Aubert I, Araujo D M, Cecyre D, Robitaille Y, Gauthier S, Quirion R. Comparative alterations of nicotinic and muscarinic binding sites in Alzheimer's and Parkinson's diseases. J Neurochem. 1992; 58:529-541.
Ashburner J, Friston K J. High-dimensional image warping. In: Frackowiak R, Ashburner J, Penny W D, et al., eds. Human Brain Function. 2nd ed. San Diego, Calif.: Academic Press; 2004:673-694.
Ashburner J, Friston K J. Rigid body registration. In: Frackowiak R, Ashburner J, Penny W D, et al., eds. Human Brain Function. 2nd ed. San Diego, Calif.: Academic Press; 2004:635-654.
Biton, B.; Bergis, O. E.; Galli, F.; Nedelec, A.; Lochead, A. W.; Jegham, S.; Godet, D.; Lanneau, C.; Santamaria, R.; Chesney, F.; Leonardon, J.; Granger, P.; Debono, M. W.; Bohme, G. A.; Sgard, F.; Besnard, F.; Graham, D.; Coste, A.; Oblin, A.; Curet, O.; Vige, X.; Voltz, C.; Rouquier, L.; Souilhac, J.; Santucci, V.; Gueudet, C.; Francon, D.; Steinberg, R.; Griebel, G.; Oury-Donat, F.; George, P.; Avenet, P.; Scatton, B. SSR180711, a novel selective alpha7 nicotinic receptor partial agonist: (1) binding and functional profile. Neuropsychopharmacology 2007, 32, 1-16.
Breese C R, Adams C, Logel J, et al. Comparison of the regional expression of nicotinic acetylcholine receptor alpha7 mRNA and [¹²⁵I]-alpha-bungarotoxin binding in human postmortem brain. J Comp Neurol. 1997; 387:385-398.
Brust, P.; Peters, D.; Deuther-Conrad, W. Development of radioligands for the imaging of alpha7 nicotinic acetylcholine receptors with positron emission tomography. Curr. Drug Targets 2012, 13, 594-601.
Brust P, Deuther-Conrad W. Molecular imaging of alpha7 nicotinic acetylcholine receptors in vivo: current status and perspectives. In: Bright P, ed. Neuroimaging: Clinical Applications. Rijeka, Croatia: InTech; 2012:533-558. Clark, J. H.; Wails, D. Fluorodenitration of activated diphenyl sulphones using tetramethylammonium fluoride. J. Fluorine Chem. 1995, 70, 201-205.
Clarke, P. B.; Schwartz, R. D.; Paul, S. M.; Pert, C. B.; Pert, A. Nicotinic binding in rat brain: autoradiographic comparison of [³H]acetylcholine, [³H]nicotine, and [¹²⁵I]-alpha-bungarotoxin. J. Neurosci. 1985, 5, 1307-1315.
Dani, J. A.; Bertrand, D. Nicotinic acetylcholine receptors and nicotinic cholinergic mechanisms of the central nervous system. Annu. Rev. Pharmacol. Toxicol. 2007, 47, 699-729.
Davies, A. R.; Hardick, D. J.; Blagbrough, I. S.; Potter, B. V.; Wolstenholme, A. J.; Wonnacott, S. Characterisation of the binding of [3H] methylcaconitine: a new radioligand for labeling alpha-type neuronal nicotinic acetylcholine receptors. Neuropharmacology 1999, 38, 679-90.
Deuther-Conrad, W.; Fischer, S.; Hiller, A.; Becker, G.; Cumming, P.; Xiong, G.; Funke, U.; Sabri, O.; Peters, D.; Brust, P. Assessment of alpha7 nicotinic acetylcholine receptor availability in juvenile pig brain with [¹⁸F]NS10743. Eur. J. Nucl. Med. Mol. Imaging 2011, 38, 1541-1549.
Ding, M.; Ghanekar, S.; Elmore, C. S.; Zysk, J. R.; Werkheiser, J. L.; Lee, C. M.; Liu, J.; Chhajlani, V.; Maier, D. L. [³H]Chiba-1001 (methyl-SSR180711) has low in vitro binding affinity and poor in vivo selectivity to nicotinic alpha-7 receptor in rodent brain. Synapse 2012, 66, 315-322.
D'Hoedt, D.; Bertrand, D. Nicotinic acetylcholine receptors: an overview on drug discovery. Expert Opin. Ther. Targets 2009, 13, 395-411.
Dolle, F.; Valette, H.; Hinnen, F.; Vaufrey, F.; Demphel, S.; Coulon, C.; Ottaviani, M.; Bottlaender, M.; Crouzel, C. Synthesis and preliminary evaluation of a carbon-11-labelled agonist of the α7 nicotinic acetylcholine receptor. J. Labelled Compd. Radiopharm. 2001, 44, 785-795.
Eckelman, W. C.; Reba, R. C.; Gibson, R. E. Receptor-binding radiotracers: a class of potential radiopharmaceuticals. J. Nucl. Med. 1979, 20, 350-357.
Ettrup, A.; Mikkelsen, J. D.; Lehel, S.; Madsen, J.; Nielsen, E. O.; Palner, M.; Timmermann, D. B.; Peters, D.; Knudsen, G. M. 11CNS14492 as a novel PET radioligand for imaging cerebral alpha7 nicotinic acetylcholine receptors: in vivo evaluation and drug occupancy measurements. J. Nucl. Med. 2011, 52, 1449-1456.
Foster D M (1998) Developing and testing integrated multicompartment models to describe a single-input multiple-output study using the SAAM II software system. Adv Exp Med Biol 445:59-78.
Fischl B, van der Kouwe A, Destrieux C et al (2004) Automatically parcellating the human cerebral cortex. Cereb Cortex 14:11-22
Gao, Y.; Ravert, H. T.; Valentine, H.; Scheffel, U.; Finley, P.; Wong, D. F.; Dannals, R. F.; Horti, A. G. 5-(5-(6-[(11)C]methyl-3,6-diazabicyclo[3.2.0]heptan-3-yl)pyridin-2-yl)-1H-indole as a potential PET radioligand for imaging cerebral alpha7-nAChR in mice. Bioorg. Med. Chem. 2012, 20, 3698-3702.
Gao, Y.; Kellar, K. J.; Yasuda, R. P.; Tran, T.; Xiao, Y.; Dannals, R. F.; Horti, A. G. Derivatives of Dibenzothiophene for Positron Emission Tomography Imaging of alpha7-Nicotinic Acetylcholine Receptors. J Med Chem 2013.
Gotti, C.; Clementi, F. Neuronal nicotinic receptors: from structure to pathology. Prog. Neurobiol. 2004, 74, 363-396.
Han Z Y, Zoli M, Cardona A, Bourgeois J P, Changeux J P, Le Novere N. Localization of [³H]nicotine, [₃H]cytisine, [³H]epibatidine, and [¹²⁵I]alpha-bungarotoxin binding sites in the brain of Macaca mulatta. J Comp Neurol. 2003; 461: 49-60.
Hashimoto, K.; Nishiyama, S.; Ohba, H.; Matsuo, M.; Kobashi, T.; Takahagi, M.; Iyo, M.; Kitashoji, T.; Tsukada, H. [11C]CHIBA-1001 as a novel PET ligand for alpha7 nicotinic receptors in the brain: a PET study in conscious monkeys. PLoS One 2008, 3, e3231.
Hashimoto, K.; Ishima, T.; Fujita, Y.; Matsuo, M.; Kobashi, T.; Takahagi, M.; Tsukada, H.; Iyo, M. Phencyclidine-induced cognitive deficits in mice are improved by subsequent subchronic administration of the novel selective alpha7 nicotinic receptor agonist SSR180711. Biol. Psychiatry 2008, 63, 92-97.
Hoffmeister, P. G.; Donat, C. K.; Schuhmann, M. U.; Voigt, C.; Walter, B.; Nieber, K.; Meixensberger, J.; Bauer, R.; Brust, P. Traumatic brain injury elicits similar alterations in alpha7 nicotinic receptor density in two different experimental models. NeuroMol. Med. 2011, 13, 44-53.
Horti, A. G.; Gao, Y.; Kuwabara, H.; Wang, Y.; Abazyan, S.; Yasuda, R. P.; Tran, T.; Xiao, Y.; Sahibzada, N.; Holt, D. P.; Kellar, K. J.; Pletnikov, M.; Pomper, M.; Wong, D. F.; Dannals, R. F. [18]ASEM ([18F]JHU82132), a radiolabeled antagonist for imaging the α7-nicotinic acetylcholine receptor (α7-nAChR) with positron emission tomography (PET). J Nucl Med, 2013.
Horti, A. G.; Ravert, H. T.; Gao, Y.; Holt, D. P.; Bunnelle, W. H.; Schrimpf, M. R.; Li, T.; Ji, J.; Valentine, H.; Scheffel, U.; Kuwabara, H.; Wong, D. F.; Dannals, R. F. Synthesis and evaluation of new radioligands [11C]A-833834 and [11C]A-752274 for positron-emission tomography of alpha7-nicotinic acetylcholine receptors. Nucl. Med. Biol. 2013, 40, 395-402.
Horti, A. G.; Villemagne, V. L. The quest for Eldorado: development of radioligands for in vivo imaging of nicotinic acetylcholine receptors in human brain. Curr. Pharm. Des. 2006, 12, 3877-3900.
Horti A G, Gao Y, Kuwabara H et al (2014) 18F-ASEM, a radiolabeled antagonist for imaging the alpha7-Nicotinic acetylcholine receptor with PET. J Nucl Med 55:672-677
Patenaude B, Smith S M, Kennedy D N, Jenkinson M (2011) A Bayesian model of shape and appearance for subcortical brain segmentation. Neuroimage 56:907-922
Hudlicky, M.; Pavlath, A. E. Chemistry of Organic Fluorine Compounds II: A Critical Review; Americal Chemical Society: Washington, D C, 1995; pp 286-289.
Innis, R. B.; Cunningham, V. J.; Delforge, J.; Fujita, M.; Gjedde, A.; Gunn, R. N.; Holden, J.; Houle, S.; Huang, S. C.; Ichise, M.; Iida, H.; Ito, H.; Kimura, Y.;
Ishikawa, M.; Hashimoto, K. alpha7 nicotinic acetylcholine receptor as a potential therapeutic target for schizophrenia. Curr. Pharm. Des. 2011, 17, 121-129.
Jenkinson M, Beckmann C F, Behrens T E, Woolrich M W, Smith S M (2012) Fsl. Neuroimage 62:782-790.
Keller S H, Sibomana M, Olesen O V et al (2012) Methods for motion correction evaluation using 18F-FDG human brain scans on a high-resolution PET scanner. J Nucl Med 53:495-504.
Kelso M L, Oestreich J H. Traumatic brain injury: central and peripheral role of alpha7 nicotinic acetylcholine receptors. Curr Drug Targets. 2012; 13:631-636. 6.
Koeppe, R. A.; Knudsen, G. M.; Knuuti, J.; Lammertsma, A. A.; Laruelle, M.; Logan, J.; Maguire, R. P.; Mintun, M. A.; Morris, E. D.; Parsey, R.; Price, J. C.; Slifstein, M.; Sossi, V.; Suhara, T.; Votaw, J. R.; Wong, D. F.; Carson, R. E. Consensus nomenclature for in vivo imaging of reversibly binding radioligands. J. Cereb. Blood Flow Metab. 2007, 27, 1533-1539.
Kubinyi, H. The Quantitative Analysis of Structure—Activity Relationships. In Burger's Medicinal Chemistry and Drug Discovery; Wolff, M. E., Ed.; John Wiley & Sons: New York, 1995; pp 497-571.
Kulak, J. M.; Schneider, J. S. Differences in alpha7 nicotinic acetylcholine receptor binding in motor symptomatic and asymptomatic MPTP-treated monkeys. Brain Res. 2004, 999, 193-202.
Kulak, J. M.; Carroll, F. I.; Schneider, J. S. [¹²⁵I]-Iodomethyllycaconitine binds to alpha7 nicotinic acetylcholine receptors in monkey brain. Eur. J. Neurosci. 2006, 23, 2604-2610.
Kuwabara H, Wong D F, Gao Y, et al. PET Imaging of nicotinic acetylcholine receptors in baboons with ¹⁸F-AZAN, a radioligand with improved brain kinetics. J Nucl Med. 2012; 53:121-129.
Lassen N A, Bartenstein P A, Lammertsma A A, et al. Benzodiazepine receptor quantification in vivo in humans using [¹¹C]flumazenil and PET: application of the steady-state principle. J Cereb Blood Flow Metab. 1995; 15:152-165.
Logan J, Fowler J S, Volkow N D, et al. Graphical analysis of reversible radioligand binding from time-activity measurements applied to [N-11 C-methyl]-(−)-cocaine PET studies in human subjects. J Cereb Blood Flow Metab. 1990; 10:740-747.
Logan J, Fowler J S, Volkow N D et al (1996) Distribution volume ratios without blood sampling from graphical analysis of PET data. J Cereb Blood Flow Metab 16:834-840.
Lukas, R. J.; Changeux, J. P.; Le Novere, N.; Albuquerque, E. X.; Balfour, D. J.; Berg, D. K.; Bertrand, D.; Chiappinelli, V. A.; Clarke, P. B.; Collins, A. C.; Dani, J. A.; Grady, S. R.; Kellar, K. J.; Lindstrom, J. M.; Marks, M. J.; Quik, M.; Taylor, P. W.; Wonnacott, S. International Union of Pharmacology. XX. Current status of the nomenclature for nicotinic acetylcholine receptors and their subunits. Pharmacol. Rev. 1999, 51, 397-401. Maier, D. L.; Hill, G.; Ding, M.; Tuke, D.; Einstein, E.; Gurley, D.; Gordon, J. C.; Bock, M. J.; Smith, J. S.; Bialecki, R.; Eisman, M.; Elmore, C. S.; Werkheiser, J. L. Pre-clinical validation of a novel alpha-7 nicotinic receptor radiotracer, [³H]AZ11637326: target localization, biodistribution and ligand occupancy in the rat brain. Neuropharmacology 2011, 61, 161-171.
Manna, S.; Maity, S.; Rana, S.; Agasti, S.; Maiti, D. ipso-Nitration of arylboronic acids with bismuth nitrate and perdisulfate. Org. Lett. 2012, 14, 1736-1739.
Marutle, A.; Zhang, X.; Court, J.; Piggott, M.; Johnson, M.; Perry, R.; Perry, E.; Nordberg, A. Laminar distribution of nicotinic receptor subtypes in cortical regions in schizophrenia. J. Chem. Neuroanat. 2001, 22, 115-126.
Mazurov, A. A.; Speake, J. D.; Yohannes, D. Discovery and development of alpha7 nicotinic acetylcholine receptor modulators. J. Med. Chem. 2011, 54, 7943-7961.
Meyer, E. M.; Kuryatov, A.; Gerzanich, V.; Lindstrom, J.; Papke, R. L. Analysis of 3-(4-hydroxy, 2-methoxybenzylidene)anabaseine selectivity and activity at human and rat alpha-7 nicotinic receptors. J. Pharmacol. Exp. Ther. 1998, 287, 918-925.
Miller, P. W.; Long, N. J.; Vilar, R.; Gee, A. D. Synthesis of ¹¹C ¹⁸F, ¹⁵O, and ¹³N radiolabels for positron emission tomography. Angew. Chem., Int. Ed. 2008, 47, 8998-9033.
Mintun, M. A.; Raichle, M. E.; Kilbourn, M. R.; Wooten, G. F.; Welch, M. J. A quantitative model for the in vivo assessment of drug binding sites with positron emission tomography. Ann. Neurol. 1984, 15, 217-227.
Muller, K.; Faeh, C.; Diederich, F. Fluorine in pharmaceuticals: looking beyond intuition. Science 2007, 317, 1881-1886.
Nag, M.; Jenks, W. S. Photochemistry of substituted dibenzothiophene oxides: the effect of trapping groups. J. Org. Chem. 2005, 70, 3458-3463.
Navarro, H. A.; Zhong, D.; Abrahm, P.; Xu, H.; Carroll, F. I. Synthesis and pharmacological characterization of ([¹²⁵I]-iodomethyllycaconitine ([¹²⁵I]iodo-MLA). A new ligand for the α7 nicotinic acetylcholine receptor. J. Med. Chem. 2000, 43, 142-145.
Ogawa, M.; Nishiyama, S.; Tsukada, H.; Hatano, K.; Fuchigami, T.; Yamaguchi, H.; Matsushima, Y.; Ito, K.; Magata, Y. Synthesis and evaluation of new imaging agent for central nicotinic acetylcholine receptor alpha7 subtype. Nucl. Med. Biol. 2010, 37, 347-355.
Olincy, A.; Harris, J. G.; Johnson, L. L.; Pender, V.; Kongs, S.; Allensworth, D.; Ellis, J.; Zerbe, G. O.; Leonard, S.; Stevens, K. E.; Stevens, J. O.; Martin, L.; Adler, L. E.; Soti, F.; Kem, W. R.; Freedman, R. Proof-of-concept trial of an alpha7 nicotinic agonist in schizophrenia. Arch. Gen. Psychiatry 2006, 63, 630-638.
Olincy A, Freedman R (2012) Nicotinic mechanisms in the treatment of psychotic disorders: a focus on the alpha7 nicotinic receptor. Handb Exp Pharmacol 213:211-232
Pani, H. R.; Hernandez, C. M.; Dineley, K. T. Research update: alpha7 nicotinic acetylcholine receptor mechanisms in Alzheimer's disease. Biochem. Pharmacol. 2011, 82, 931-942.
Peters D, Timmerman D B, Roenn L C, Nielsen E B (2009) Preparation of labeled indolyl-pyridazinyl-diazabicyclononane derivatives and their use in diagnostic methods, particularly receptor imaging.
Philip, N. S.; Carpenter, L. L.; Tyrka, A. R.; Price, L. H. Nicotinic acetylcholine receptors and depression: a review of the preclinical and clinical literature. Psychopharmacology (Berlin, Ger.) 2010, 212, 1-12.
Pichat, P.; Bergis, O. E.; Terranova, J. P.; Urani, A.; Duarte, C.; Santucci, V.; Gueudet, C.; Voltz, C.; Steinberg, R.; Stemmelin, J.; Oury-Donat, F.; Avenet, P.; Griebel, G.; Scatton, B. SSR180711, a novel selective alpha7 nicotinic receptor partial agonist: (II) efficacy in experimental models predictive of activity against cognitive symptoms of schizophrenia. Neuropsychopharmacology 2007, 32, 17-34.
Pomper, M. G.; Phillips, E.; Fan, H.; McCarthy, D. J.; Keith, R. A.; Gordon, J. C.; Scheffel, U.; Dannals, R. F.; Musachio, J. L. Synthesis and biodistribution of radiolabeled alpha 7 nicotinic acetylcholine receptor ligands. J. Nucl. Med. 2005, 46, 326-334.
Prickaerts J, van Goethem N P, Chesworth R et al (2012) EVP-6124, a novel and selective alpha7 nicotinic acetylcholine receptor partial agonist, improves memory performance by potentiating the acetylcho-line response of alpha7 nicotinic acetylcholine receptors. Neuropharmacology 62:1099-1110.
Ravert, H. T.; Dorff, P.; Foss, C. A.; Mease, R. C.; Fan, H.; Holmquist, C. R.; Phillips, E.; McCarthy, D. J.; Heys, J. R.; Holt, D. P.; Wang, Y.; Endres, C. J.; Dannals, R. F.; Pomper, M. G. Radiochemical synthesis and in vivo evaluation of [¹⁸F]AZ11637326: an agonist probe for the alpha7 nicotinic acetylcholine receptor. Nucl. Med. Biol. 2013, 40, 731-739.
Reagan-Shaw S, Nihal M, Ahmad N (2008) Dose translation from animal to human studies revisited. FASEB J 22:659-661.
Rollema H, Shrikhande A, Ward K M et al (2010) Pre-clinical properties of the alpha4beta2 nicotinic acetylcholine receptor partial agonists varenicline, cytisine and dianicline translate to clinical efficacy for nicotine dependence. Br J Pharmacol 160:334-345.
Rollema, H.; Shrikhande, A.; Ward, K. M.; Tingley, F. D., 3rd; Coe, J. W.; O'Neill, B. T.; Tseng, E.; Wang, E. Q.; Mather, R. J.; Hurst, R. S.; Williams, K. E.; de Vries, M.; Cremers, T.; Bertrand, S.; Bertrand, D. Pre-clinical properties of the alpha4beta2 nicotinic acetylcholine receptor partial agonists varenicline, cytisine and dianicline translate to clinical efficacy for nicotine dependence. Br. J. Pharmacol. 2010, 160, 334-345.
Rotering, S.; Scheunemann, M.; Fischer, S.; Hiller, A.; Peters, D.; Deuther-Conrad, W.; Brust, P. Radiosynthesis and first evaluation in mice of [¹⁸F]NS14490 for molecular imaging of alpha7 nicotinic acetylcholine receptors. Bioorg. Med. Chem. 2013, 21, 2635-2642.
Schrimpf, M. R.; Sippy, K. B.; Briggs, C. A.; Anderson, D. J.; Li, T.; Ji, J.; Frost, J. M.; Surowy, C. S.; Bunnelle, W. H.; Gopalakrishnan, M.; Meyer, M. D. SAR of alpha7 nicotinic receptor agonists derived from tilorone: exploration of a novel nicotinic pharmacophore. Bioorg. Med. Chem. Lett. 2012, 22, 1633-1638.
Smith, M. B.; March, J. March's Advanced Organic Chemistry; John Wiley & Sons, Inc.: Hoboken, N. J., 2007; pp 853-869.
Stabin M G, Siegel J A (2003) Physical models and dose factors for use in internal dose assessment. Health Phys 85:294-310
Stabin M G, Sparks R B, Crowe E (2005) OLINDA/EXM: the second-generation personal computer software for internal dose assessment in nuclear medicine. J Nucl Med 46:1023-1027.
Taly, A.; Charon, S. alpha7 nicotinic acetylcholine receptors: a therapeutic target in the structure era. Curr. Drug Targets 2012, 13, 695-706.
Tanibuchi, Y.; Wu, J.; Toyohara, J.; Fujita, Y.; Iyo, M.; Hashimoto, K. Characterization of [³H]CHIBA-1001 binding to alpha7 nicotinic acetylcholine receptors in the brain from rat, monkey, and human. Brain Res. 2010, 1348, 200-208.
Thomsen, M. S.; Hansen, H. H.; Timmerman, D. B.; Mikkelsen, J. D. Cognitive improvement by activation of alpha7 nicotinic acetylcholine receptors: from animal models to human pathophysiology. Curr. Pharm. Des. 2010, 16, 323-343.
Thomsen M S, Weyn A, Mikkelsen J D (2011) Hippocampal alpha7 nicotinic acetylcholine receptor levels in patients with schizophrenia, 7589 bipolar disorder, or major depressive disorder. Bipolar Disord 13:701-707
Tichauer, K. M.; Samkoe, K. S.; Sexton, K. J.; Hextrum, S. K.; Yang, H. H.; Klubben, W. S.; Gunn, J. R.; Hasan, T.; Pogue, B. W. In vivo quantification of tumor receptor binding potential with dualreporter molecular imaging. Mol. Imaging Biol. 2011, 14, 584-592.
Toyohara, J.; Ishiwata, K.; Sakata, M.; Wu, J.; Nishiyama, S.; Tsukada, H.; Hashimoto, K. In vivo evaluation of alpha7 nicotinic acetylcholine receptor agonists [11C]A-582941 and [11C]A-844606 in mice and conscious monkeys. PLoS One 2010, 5, e8961.
Toyohara, J.; Sakata, M.; Wu, J.; Ishikawa, M.; Oda, K.; Ishii, K.; Iyo, M.; Hashimoto, K.; Ishiwata, K. Preclinical and the first clinical studies on [11C]CHIBA-1001 for mapping alpha7 nicotinic receptors by positron emission tomography. Ann. Nucl. Med. 2009, 23, 301-309.
Toyohara, J.; Wu, J.; Hashimoto, K. Recent development of radioligands for imaging alpha7 nicotinic acetylcholine receptors in the brain. Curr. Top. Med. Chem. 2010, 10, 1544-1557.
Verbois, S. L.; Sullivan, P. G.; Scheff, S. W.; Pauly, J. R. Traumatic brain injury reduces hippocampal alpha7 nicotinic cholinergic receptor binding. J. Neurotrauma 2000, 17, 1001-1011.
Verbois, S. L.; Scheff, S. W.; Pauly, J. R. Time-dependent changes in rat brain cholinergic receptor expression after experimental brain injury. J. Neurotrauma 2002, 19, 1569-1585.
Woodruff-Pak, D. S.; Gould, T. J. Neuronal nicotinic acetylcholine receptors: involvement in Alzheimer's disease and schizophrenia. Behav. Cognit. Neurosci. Rev. 2002, 1, 5-20.
Zhang, L.; Villalobos, A.; Beck, E. M.; Bocan, T.; Chappie, T. A.; Chen, L.; Grimwood, S.; Heck, S. D.; Helal, C. J.; Hou, X.; Humphrey, J. M.; Lu, J.; Skaddan, M. B.; McCarthy, T. J.; Verhoest, P. R.; Wager, T. T.; Zasadny, K. Design and selection parameters to accelerate the discovery of novel central nervous system positron emission tomography (PET) ligands and their application in the development of a novel phosphodiesterase 2A PET ligand. J. Med. Chem. 2013, 56, 4568-4579.
Wallace, T. L.; Bertrand, D. Alpha7 neuronal nicotinic receptors as a drug target in schizophrenia. Expert Opin. Ther. Targets 2013, 17, 139-155.
Whiteaker, P.; Davies, A. R.; Marks, M. J.; Blagbrough, I. S.; Potter, B. V.; Wolstenholme, A. J.; Collins, A. C.; Wonnacott, S. An autoradiographic study of the distribution of binding sites for the novel alpha7-selective nicotinic radioligand [³H]-methyllycaconitine in the mouse brain. Eur. J. Neurosci. 1999, 11, 2689-96.
Xiao, Y.; Abdrakhmanova, G. R.; Baydyuk, M.; Hernandez, S.; Kellar, K. J. Rat neuronal nicotinic acetylcholine receptors containing alpha7 subunit: pharmacological properties of ligand binding and function. Acta Pharmacol. Sin. 2009, 30, 842-850.
Xiao, Y.; Kellar, K. The comparative pharmacology and upregulation of rat neuronal nicotinic receptor subtype binding sites stably expressed in transfected mammalian cells. J. Pharmacol. Exp. Ther. 2004, 310, 98-107.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A non-invasive method for imaging one or more α7-nicotinic acetylcholine receptors (α7-nAChRs) in the brain of a subject, the method comprising:
administering to the subject an effective amount of a radiolabeled compound of Formula (I) 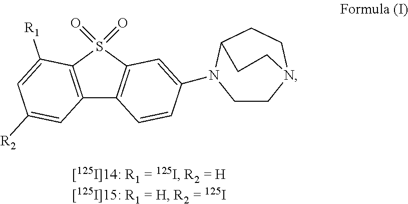
or a pharmaceutically acceptable salt, hydrate or prodrug thereof;
allowing the radiolabeled compound to bind to the α7-nAChRs in the brain of the subject; and
obtaining an image of the α7-nAChRs in the brain of the subject.

2. The method of claim 1, wherein the image is obtained by using single-photon emission computed tomography.

3. The method of claim 1, wherein the compound selectively binds to the one or more α7-nAChRs relative to other nicotinic acetylcholine receptors in the brain.

4. The method of claim 1, wherein the radiolabeled compound readily enters the brain of the subject.

5. A non-invasive method for quantifying one or more α7-nicotinic acetylcholine receptors (α7-nAChRs) in the brain of a subject, the method comprising:
administering to the subject an effective amount of a radiolabeled compound of Formula (I) 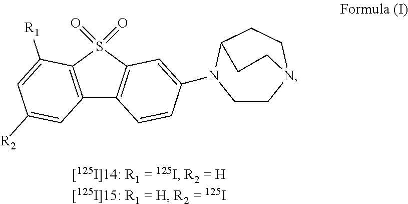
or a pharmaceutically acceptable salt, hydrate or prodrug thereof;
allowing the radiolabeled compound to bind to the one or more α7-nAChRs in the brain of the subject;
obtaining an image of the brain of the subject showing the distribution of the radiolabeled compound; and
deriving a standardized uptake value (SUV) from the image of the brain.

6. The method of claim 1, wherein the image is obtained by using single-photon emission computed tomography.

7. The method of claim 5, wherein the radiolabeled compound selectively binds to the one or more α7-nAChRs relative to other nicotinic acetylcholine receptors in the brain.

8. The method of claim 5, wherein the radiolabeled compound readily enters the brain of the subject.

9. A non-invasive method for imaging one or more α7-nicotinic acetylcholine receptors (α7-nAChRs) in the brain of a subject, the method comprising:
administering to the subject an effective amount of [¹⁸F]-ASEM compound, or a pharmaceutically acceptable salt, hydrate or prodrug thereof;
allowing the compound to bind to the one or more α7-nAChRs in the brain of the subject; and
obtaining an image of the brain of the subject using positron emission tomography, wherein the binding is reversible.

10. The method of claim 9, wherein the compound readily enters the brain of the subject.

11. The method of claim 9, wherein the specificity of the binding is at least about 80 percent.

12. The method of claim 9, wherein the compound exhibits a percentage standardized uptake value of about 400 at 10 to 15 minutes.

13. The method of claim 9, wherein the binding is reversible within approximately 90 minutes.

14. A non-invasive method for quantifying one or more α7-nicotinic acetylcholine receptors (α7-nAChRs) in the brain of a subject, the method comprising:
administering to the subject an effective amount of [¹⁸F]-ASEM compound, or a pharmaceutically acceptable salt, hydrate or prodrug thereof;
allowing the compound to bind to the one or more α7-nAChRs in the brain of the subject;
obtaining a positron emission tomography (PET) image of the brain of the subject showing the distribution of the compound; and
deriving a standardized uptake value (SUV) from the image of the brain.

15. The method of claim 14 wherein the compound readily enters the brain of the subject.

16. The method of claim 14 wherein the specificity of the binding is at least about 80 percent.

17. The method of claim 14 wherein the compound exhibits a percentage standardized uptake value of about 400 at 10 to 15 minutes.

18. The method of claim 14, wherein the binding is reversible within approximately 90 minutes.

19. A non-invasive method for diagnosing a disease or condition associated with α7-nAChRs in a subject in need thereof, the method comprising:
administering to the subject a composition comprising an effective amount of a radiolabeled compound of Formula (I), (II) or (III): 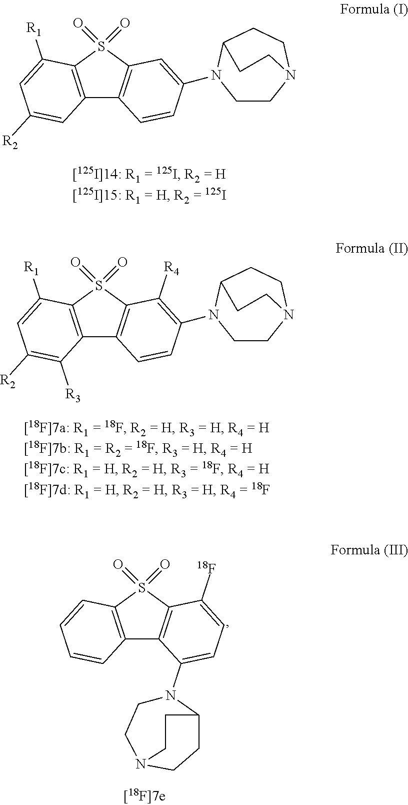
or a pharmaceutically acceptable salt, hydrate or prodrug thereof,
allowing the radiolabeled compound to bind to the α7-nAChRs in the brain of the subject; and
obtaining an imaging of the brain of the subject,
wherein an alteration in the density of α7-nAChRs in the brain as compared to the brain of a subject without the disease or condition is indicative that the subject has the disease or condition associated with α7-nAChRs.

20. The method of claim 19, wherein the disease or condition is associated with α7-nAChRs is selected from the group consisting of schizophrenia, Alzheimer's disease, Parkinson's disease, anxiety, depression, attention deficit hyperactivity disorder (ADHD), multiple sclerosis, cancer, macrophage chemotaxis, inflammation, traumatic brain injury and drug addiction.

21. The method of claim 19, wherein the radiolabeled compound readily enters the brain of the subject.

22. The method of claim 19, wherein the radiolabeled compound is selected from the group consisting of 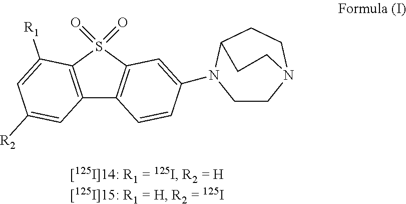
and the image is obtained by using single-photon emission computed tomography.

23. The method of claim 19, wherein the compound selectively binds to the α7-nAChRs relative to other nicotinic acetylcholine receptors.

24. The method of claim 19, wherein the radiolabeled compound is selected from the group consisting of 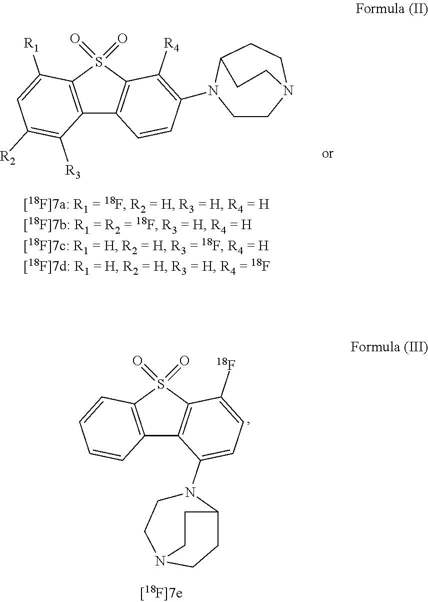
and the image is obtained by positron emission tomography.

25. The method of claim 24, wherein the radiolabeled compound is [¹⁸F]-ASEM.

26. The method of claim 24, wherein the specificity of the binding is at least 80 percent.

27. The method of claim 24, wherein the radiolabeled compound exhibits a percentage standardized uptake value of about 400 at 10 to 15 minutes.

28. The method of claim 24, wherein the binding is reversible within approximately 90 minutes.

29. A method for preparing a compound of Formula (I): 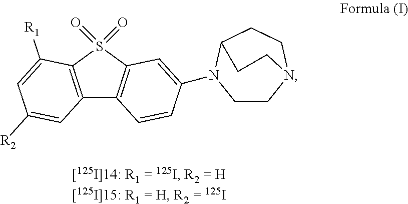
the method comprising:
(a) contacting a solution of a compound of Formula (IV) 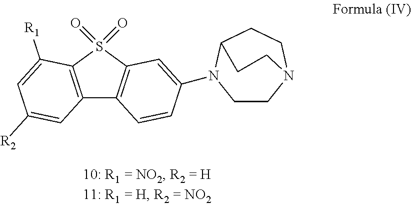
in a solvent with Na ¹²⁵I to form a mixture;
(b) adding an acid to the mixture;
(c) heating the mixture;
(d) cooling the mixture;
(e) diluting the mixture in an appropriate solvent;
(f) applying the diluted mixture to a reverse phase HPLC column;
(g) collecting the radioactive peak;
(h) transferring the radioactive peak to a solid phase extraction (SPE) cartridge;
(i) eluting the product through a filter; and
(j) adding saline and a solution of sodium bicarbonate through the filter to form Formula (I).

30. The method of claim 29, wherein the solvent used in step (a) is CH₃CN.

31. The method of claim 29, wherein step (a) is carried out at room temperature.

32. The method of claim 29, wherein the acid used in step (b) is TFA.

33. The method of claim 29, wherein the solvent used in step (e) is CH₃CN.

34. The method of claim 29, wherein the SPE in step (h) is washed with saline.

35. The method of claim 29, wherein the elution buffer comprises ethanol and HCl.

36. The method of claim 29, wherein the filter has a pore size of about 0.2-μm.

37. A compound of Formula (I): 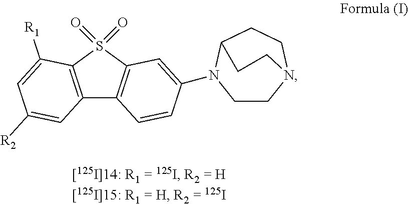
or a pharmaceutically acceptable salt, hydrate or prodrug thereof.
